(19) United States
(12) Patent Application Publication
(10) Pub. No.: US 2012/0128642 A1
(43) Pub. Date: May 24, 2012

(54) POTENCY MARKERS

(76) Inventors: Jeffrey Keeler Teumer, Brookline, MA (US); Vladimir Mastyugin, Lexington, MA (US); Jizeng Qiao, Lexington, MA (US); Agatha Zawadzka, Essex, MA (US)

(21) Appl. No.: 13/386,263

(22) PCT Filed: Jul. 23, 2010
(86) PCT No.: PCT/US2010/043042

§ 371 Date: Jan. 20, 2012

Related U.S. Application Data
(60) Provisional application No. 61/227,964, filed on Jul. 23, 2009.

(51) Int. Cl. A61K 35/36; G01N 21/64; C40B 30/04; C12N 5/071; A61P 17/14; G01N 33/566; C12Q 1/68

(52) U.S. Cl. 424/93.7; 435/7.1; 435/7.21; 435/7.9; 435/6.11; 435/375; 506/9

(57) ABSTRACT

Methods for identifying trichogenic dermal cells, including dermal papilla cells and dermal sheath cells, capable of inducing hair follicle formation when injected into skin are provided. Biomarkers have been discovered that can be used to detect, identify, and distinguish trichogenic dermal cells, i.e., that are able to induce hair follicle formation, from non-trichogenic skin cells. Populations of enriched trichogenic dermal cells can be produced by selecting for and enriching for dermal cells that the disclosed biomarkers. These enriched trichogenic dermal can be used for inducing hair follicle formation.

### CROSS REFERENCE TO RELATED APPLICATIONS

This application claims benefit of U.S. Provisional Application No. 61/227,964, filed Jul. 23, 2009, which is hereby incorporated herein by reference in its entirety.

### FIELD OF THE INVENTION

The invention is generally related to the field of hair transplantation, more particularly to biomarkers and methods for the identification and/or isolation of trichogenic dermal cells, such as dermal papilla (DP) cells and dermal sheath (DS) cells.

### BACKGROUND OF THE INVENTION

Hair loss or alopecia is a common problem in both males and females regardless of their age. There are several types of hair loss, such as androgenetic alopecia, alopecia areata, telogen effluvium, hair loss due to systemic medical problems, e.g., thyroid disease, adverse drug effects and nutritional deficiency states as well as hair loss due to scalp or hair trauma, discoid lupus erythematosus, lichen planus and structural shaft abnormalities. (Hogan and Chamberlain, South Med J, 93(7):657-62 (2000)). Androgenetic alopecia is the most common cause of hair loss, affecting about 50% of individuals who have a strong family history of hair loss. Androgenetic alopecia is caused by three interdependent factors: male hormone dihydrotestosterone (DHT), genetic disposition and advancing age. DHT causes hair follicles to degrade and further shrink in size, resulting in weak hairs. DHT also shortens the anagen phase of the hair follicle growing cycle. Over time, more hairs are shed and hairs become thinner.

Possible options for the treatment of alopecia include hair prosthesis, surgery and topical/oral medications. (Hogan & Chamberlain, 2000; Bertolino, J Dermatol, 20(10):604-10 (1993)). While drugs such as minoxidil, finasteride and dutasteride represent significant advances in the management of male pattern hair loss, the fact that their action is temporary and the hairs are lost after stopping therapy continues to be a major limitation (Bouhanna, Dermatol Surg, 28:136-42 (2002); Avram, et al., Dermatol Surg, 28:894-900 (2002)). In view of this, surgical hair restoration and tissue engineering may be the only permanent methods of treating pattern baldness. The results from surgical hair transplantation can vary and early punch techniques often resulted in a highly unnatural “doll hair look” or “paddy field look” over the recipient area. Although advances have been made in surgical hair transplantation, for example, using single follicle hair grafts with 1 mm punches, the procedures are time consuming and costly and most important, the number of donor follicles on a given patient is limited.

Tissue engineering to treat hair loss includes transplanting cells into an area to induce hair follicle formation and subsequent hair shaft formation. Theoretically, this simple but effective method of tissue engineering may be employed to treat hair loss due to a variety of diseases, syndromes, and injuries and may provide significant insights into tissue and organ engineering. Hair follicle induction and growth involves active and continuous epithelial and mesenchymal interactions (Stenn & Paus, Physiol Reviews, 81:449-494, (2001)). In the embryo, the first hair follicles grow from a thickening of the primitive epidermis by signals arising from dermal cells. Early studies (Cohen, J Embryol Exp Morphol, 9:117-127 (1961)) using adult rodent hair follicles showed that the dissected deep mesenchymal portion of the hair follicle, the follicular or dermal papilla, when implanted under adult epidermis, will induce new hair follicles. This powerful inductive property is ascribed to a unique property of the cells in the papilla and about the base of the follicle—the dermal sheath (McElwee et al., J Invest Dermatol, 121:1267-1275 (2003)). Dermal papilla (DP) cells and dermal sheath (DS) cells from adult hair follicles can therefore be used to regenerate new hair follicles, i.e., are trichogenic dermal cells. Later work by Jahoda et al. (1984, Nature 311: 560-562) demonstrated that cultured DP cells can also induce hair follicle formation, raising the possibility that cultured DP cells and/or cultured DS cells could be used for hair regeneration or restoration in the cosmetic or therapeutic treatment of androgenetic alopecia and other hair loss disorders.

However, in order to be effective for hair regeneration, cultured DP cells and/or cultured DS cells need to maintain their hair-inductive capacity. DP cells and/or DS cells in culture will lose this capacity unless special culture conditions are employed, as described by Jahoda et al. (1984, above), Messenger (1984, Br J Dermatol 110: 685-689), Matsuzaki et al. (1996, In: Hair Research for the Next Millenium, Van Neste & Randall (Eds), Elsevier Science, New York, 447-451) and Kishimoto et al. (2000, Genes Dev 14: 1181-1185). Loss of the hair inductive capability cannot be determined by cursory examination of cultures because DP cells and/or DS cells that are no longer capable of hair induction have morphologic and growth properties apparently identical to those of DP cells and/or DS cells that are capable of hair induction.

At present, the only methods available to determine if cultures of DP cells and/or DS cells are capable of hair induction are in vivo grafting methods, typically wherein the cells are implanted into rodents to determine if a hair is formed. These methods require large numbers of cells and take several weeks to carry out, and they do not yield quantitative measurements of hair inductive potency.

Therefore, it is an object of the invention to provide biomarkers for identifying and enriching dermal cells, such as DP cells and DS cells, that are capable of inducing hair follicle formation (i.e., trichogenic dermal cells).

It is another object of the invention to provide cell populations enriched with trichogenic dermal cells.

It is another object of the invention to provide methods and compositions for treating hair loss in a subject.

### SUMMARY OF THE INVENTION

Methods for identifying dermal cells capable of inducing hair follicle formation when injected into skin are provided. It has been discovered that expression of Serglycin (SRGN), Src-like-adaptor—encoded polypeptide 3 (SLA), Thrombomodulin (THBD), Runt-related transcription factor 2 (RUNX2), Runt-related transcription factor 3 (RUNX3), Protocadherin 17 (PCDH17), Lymphocyte antigen 75 (LY75), Placental Growth Factor (PGF), Amyloid beta (A4) precursor protein-binding, family A, member 2 (APBA2), Prostaglandin E synthase (PTGES), myosin IF (MYO1F), G protein-coupled receptor 84 (GPR84), Transcription elongation factor A (SII)-like 2 (TCEAL2), Collagen, type XXIII, alpha 1 (COL23A1), ST8 alpha-N-acetyl-neuraminide alpha-2,8-sialyltransferase 4 (ST8SIA4), Matrix metallopeptidase 8 (MMP8), Developmental pluripotency associated 4 (DPPA4), and Endothelial cell-specific molecule 2 (ECSM2) can be used as biomarkers to detect, identify, and distinguish trichogenic dermal papilla (DP) cells and/or dermal sheath (DS) cells from non-trichogenic skin cells.

Populations of cells enriched for trichogenic DP and/or DS cells can therefore be produced by selecting for and enriching for skin cells that express one or more of the disclosed biomarkers. In some embodiments, the one or more biomarkers are detected as proteins. In some embodiments, the one or more biomarkers are detected as nucleic acids. Therefore, a population of cells enriched for trichogenic dermal cells, such as DS cells and/or DP cells expressing one or more of the disclosed biomarkers, is also provided. Skin cell populations are also provided that contain an enriched population of trichogenic dermal cells combined with epidermal cells for use in inducing hair follicle formation.

Methods for inducing hair follicle formation are also provided. These methods can involve administering to a subject a population of trichogenic dermal cells enriched for expression of SRGN, SLA, THBD, RUNX2, RUNX3, PCDH17, LY75, PGF, APBA2, PTGES, MYO1F, GPR84, TCEAL2, COL23A1, ST8SIA4, MMP8, DPPA4, ECSM2, or a combination thereof.

### BRIEF DESCRIPTION OF THE DRAWINGS

FIG. 1 is a schematic view of methodology for immunomagnetic isolation of Dermal Papilla (DP) cells using antibodies specific for a trichogenic biomarker.

### DETAILED DESCRIPTION OF THE INVENTION

### I. Definitions

To facilitate understanding of the disclosure, the following definitions are provided:

The term “trichogenic cells” refers to skin cells that induce hair follicle formation. Induction of hair follicles can be direct or indirect.

The term “skin” refers to the outer covering of an animal. In general, the skin includes the epidermis and the dermis. Skin cells can include cells in or around a hair follicle, including fibroblasts, keratinocytes, melanocytes, dermal papilla cells, dermal sheath cells, and outer root sheath cells.

The term “trichogenic dermal cells” refers to dermal cells, such as dermal papilla (DP) cells and dermal sheath (DS) cells, that induce hair follicle formation.

The term “effective amount” refers to an amount of cells needed to induce hair follicle formation.

The terms “individual”, “host”, “subject”, and “patient” are used interchangeably herein, and refer to a mammal, including, but not limited to, murines, simians, humans, mammalian farm animals, mammalian sport animals, and mammalian pets.

The term “biomarker” refers to a nucleic acid or protein whose expression or presence is indicative of trichogenic dermal cells, such as DP cells or DS cells. Representative biomarkers, include, but are not limited to Serglycin (SRGN), Src-like-adaptor—encoded polypeptide 3 (SLA), Thrombomodulin (THBD), Runt-related transcription factor 2 (RUNX2), Runt-related transcription factor 3 (RUNX3), Protocadherin 17 (PCDH17), Lymphocyte antigen 75 (LY75), Placental Growth Factor (PGF), Amyloid beta (A4) precursor protein-binding, family A, member 2 (APBA2), Prostaglandin E synthase (PTGES), myosin IF (MYO1F), G protein-coupled receptor 84 (GPR84), Transcription elongation factor A (SII)-like 2 (TCEAL2), Collagen, type XXIII, alpha 1 (COL23A1), ST8 alpha-N-acetyl-neuraminide alpha-2,8-sialyltransferase 4 (ST8SIA4), Matrix metallopeptidase 8 (MMP8), Developmental pluripotency associated 4 (DPPA4), and Endothelial cell-specific molecule 2 (ECSM2).

The term “enriched” refers to a population of cells having an increase in the percentage of a given cell relative to reference skin cell populations. For example, as used herein, “enriched trichogenic dermal cells” refers to a population of cells that contains at least 30%, 40%, 50%, 60%, 70%, 80%, 90%, 95%, 97%, 98%, 99% or 100% DP cells and/or DS cells that can induce hair follicle formation.

The term “isolated” refers to cells that are in an environment different from that in which the cells naturally occur e.g., separated from its natural milieu such as by separating dermal cells from a hair follicle.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “nucleic acid” may be used to refer to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

The term “oligonucleotide” refers to a single-stranded nucleic acid polymer of a defined sequence that can base-pair to a second single-stranded nucleic acid polymer that contains a complementary sequence.

The term “complementary” and “complementarity” refers to the rules of Watson and Crick base pairing. For example, A (adenine) bonds with T (thymine) or U (uracil), G (guanine) bonds with C (cytosine). For example, DNA contains an antisense strand that is complementary to its sense strand. A nucleic acid that is 95% identical to a DNA antisense strand is therefore 95% complementary to the DNA sense strand.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

The term “vector” refers to a replicon, such as a plasmid, phage, or cosmid, into which another DNA segment may be inserted so as to bring about the replication of the inserted segment. The vectors can be expression vectors.

The term “expression vector” refers to a vector that includes one or more expression control sequences

The term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

The term “promoter” refers to a regulatory nucleic acid sequence, typically located upstream (5′) of a gene or protein coding sequence that, in conjunction with various elements, is responsible for regulating the expression of the gene or protein coding sequence.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The term “endogenous” with regard to a nucleic acid refers to nucleic acids normally present in the host.

### II. Trichogenic Dermal Papilla Cells and Dermal Sheath Cells

A. Biomarkers for Trichogenic DP Cells and DS Cells

Biomarkers are provided that are expressed in cultured DP cells and/or DS cells that are still capable of inducing hair formation but are not expressed by cultured DP cells and/or DS cells that are no longer able to induce hair formation. Biomarkers are therefore provided that are differentially expressed by trichogenic dermal cells, such as DP cells and/or DS cells. Expression of the disclosed biomarkers in skin cells correlates with hair induction capacity, i.e., trichogenicity. Therefore, in some embodiments, these biomarkers are not detectable on non-trichogenic dermal cells, such as cultured DP cells or DS cells that have lost the ability to induce hair follicle formation, or by other skin cells, such as fibroblasts or keratinocytes. In other embodiments, these biomarkers have increased expression in trichogenic dermal cells relative to non-trichogenic cells.

Biomarkers that correlate with hair induction can be used to rapidly identify such trichogenic dermal cells, for example in cell cultures. The disclosed biological markers may also be used to quantify the number of trichogenic dermal cells in a sample of cells. The disclosed biological markers may also be used to monitor a sample of cells for maintenance of hair induction capability in culture. The disclosed biological markers may also be used to detect a trichogenic cell in a sample from a patient suffering from hair loss. The disclosed biological markers may also be used to isolate the detected trichogenic dermal cell. This isolated cell may then be used for hair restoration. In some embodiments, the isolated cell is cultured to produce a population of cells containing trichogenic dermal cells. Trichogenic cells can lose their trichogenicity in cell culture. Therefore, in these embodiments, the disclosed biomarkers can be used to further isolate trichogenic dermal cells from a population of cultured cells containing both trichogenic and non-trichogenic cells.

Isolated populations of trichogenic DP cells and/or DS cells may be obtained using the disclosed one or more biomarkers in combination with a variety of isolation methods known to skilled artisans.

In some embodiments, the trichogenic DP cells and/or DS cells are isolated from a population of skin cells isolated from a subject. In these embodiments, trichogenic dermal cells can be isolated from other skin cells, such as fibroblasts or keratinocytes. Trichogenic dermal cells can also be isolated from non-trichogenic dermal cells.

In other embodiments, the trichogenic DP cells and/or DS cells are isolated from a population of cultured DP cells and/or DS cells. In these embodiments, those DP cells and/or DS cells that have maintained trichogenicity during culture are isolated from those cells that have lost the ability to induce hair follicle formation.

Biomarkers indicative of trichogenicity include Serglycin (SRGN), Src-like-adaptor—encoded polypeptide 3 (SLA), Thrombomodulin (THBD), Runt-related transcription factor 2 (RUNX2), Runt-related transcription factor 3 (RUNX3), Protocadherin 17 (PCDH17), Lymphocyte antigen 75 (LY75), Placental Growth Factor (PGF), Amyloid beta (A4) precursor protein-binding, family A, member 2 (APBA2), Prostaglandin E synthase (PTGES), myosin IF (MYO1F), G protein-coupled receptor 84 (GPR84), Transcription elongation factor A (SII)-like 2 (TCEAL2), Collagen, type XXIII, alpha 1 (COL23A1), ST8 alpha-N-acetyl-neuraminide alpha-2,8-sialyltransferase 4 (ST8SIA4), Matrix metallopeptidase 8 (MMP8), Developmental pluripotency associated 4 (DPPA4), Endothelial cell-specific molecule 2 (ECSM2), or combinations thereof.

The disclosed biomarker can be an oligonucleotide marker or a polypeptide marker. Therefore, the disclosed biological marker can be an oligonucleotide marker having a nucleic acid sequence of any of SEQ ID NOs:1-21, or a variant or fragment of the nucleic acid marker. The disclosed biological marker can be a polypeptide marker having an amino acid sequence of any of SEQ ID NOs:22-46, or a variant or fragment of the polypeptide marker.

Serglycin is a protein that in humans is encoded by the SRGN gene. Serglycin is a hematopoietic cell granule proteoglycan. Proteoglycans stored in the secretory granules of many hematopoietic cells also contain a protease-resistant peptide core, which may be important for neutralizing hydrolytic enzymes. This encoded protein was found to be associated with the macromolecular complex of granzymes and perforin, which may serve as a mediator of granule-mediated apoptosis. Human SRGN has the following nucleic acid sequence:

(SEQ ID NO: 1)
ATTTTCTAAA AGGGACAGAG AGCACCCTGC TACATTTCCT AATCAAGAAG TTGGCGTGCA
GCTGGGAGAG CTAGACTAAG TTGGTCATGA TGCAGAAGCT ACTCAAATGC AGTCGGCTTG
TCCTGGCTCT TGCCCTCATC CTGGTTCTGG AATCCTCAGT TCAAGGTTAT CCTACGCGGA
GAGCCAGGTA CCAATGGGTG CGCTGCAATC CAGACAGTAA TTCTGCAAAC TGCCTTGAAG
AAAAAGGACC AATGTTCGAA CTACTTCCAG GTGAATCCAA CAAGATCCCC CGTCTGAGGA
CTGACCTTTT TCCAAAGACG AGAATCCAGG ACTTGAATCG TATCTTCCCA CTTTCTGAGG
ACTACTCTGG ATCAGGCTTC GGCTCCGGCT CCGGCTCTGG ATCAGGATCT GGGAGTGGCT
TCCTAACGGA AATGGAACAG GATTACCAAC TAGTAGACGA AAGTGATGCT TTCCATGACA
ACCTTAGGTC TCTTGACAGG AATCTGCCCT CAGACAGCCA GGACTTGGGT CAACATGGAT
TAGAAGAGGA TTTTATGTTA TAGAAGAGGA TTTTCCCACC TTGACACCAG GCAATGTAGT
TAGCATATTT TATGTACCAT GGTTATATGA TTAATCTTGG GACAAAGAAT TTTATAGAAA
TTTTTAAACA TCTGAAAAAG AAGCTTAAGT TTTATCATCC TTTTTTTTCT CATGAATTCT
TAAAGGATTA TGCTTTAATG CTGTTATCTA TTTTATTGTT CTTGAAAATA CCTGCATTTT
TTGGTATCAT GTTCAACCAA CATCATTATG AAATTAATTA GATTCCCATG GCCATAAAAT
GGCTTTAAAG AATATATATA TATTTTTAAA GTAGCTTGAG AAGCAAATTG GCAGGTAATA
TTTCATACCT AAATTAAGAC TCTGACTTGG ATTGTGAATT ATAATGATAT GCCCCTTTTC
TTATAAAAAC AAAAAAAAAA ATAATGAAAC ACAGTGAATT TGTAGAGTGG GGGTATTTGA
CATATTTTAC AGGGTGGAGT GTACTATATA CTATTACCTT TGAATGTGTT TGCAGAGCTA
GTGGATGTGT TTGTCTACAA GTATGATTGC TGTTACATAA CACCCCAAAT TAACTCCCAA
ATTAAAACAC AGTTGTGCTG TCAATACCTC ATACTGCTTT ACCTTTTTTT CCTGGATATC
TGTGTATTTT CAAATGTTAC TATATATTAA AGCAGAAATA TAACCAAAGG TTAAAAAAAA
AAAAAAAAAA.

Human Serglycin has the following amino acid sequence:

(SEQ ID NO: 22)
MMQKLLKCSR LVLALALILV LESSVQGYPT RRARYQWVRC
NPDSNSANCL EEKGPMFELL PGESNKIPRL RTDLFPKTRI
QDLNRIFPLS EDYSGSGFGS GSGSGSGSGS GFLTEMEQDY
QLVDESDAFH DNLRSLDRNL PSDSQDLGQH GLEEDFML.

Src-like-adaptor (SLA) is an adapter protein, which negatively regulates T-cell receptor (TCR) signaling. SLA inhibits T-cell antigen-receptor induced activation of nuclear factor of activated T-cells. SLA is involved in the negative regulation of positive selection and mitosis of T-cells. SLA may act by linking signaling proteins such as ZAP70 with CBL, leading to a CBL dependent degradation of signaling proteins. Human SLA has the following nucleic acid sequence:

(SEQ ID NO: 2)
AACCAATCTT CACCAATCTC ATCTTCACAT ATAAACAGCC
GCCTTTCAAG AAGCAAGCTG CCAGAAAAAT GATGCACGAT
GCTCTCTAAA CTGGGTCATT CTCCACTTGG AGGGCTCAGG
GCACGGTTGA CTTTCCCCGT CTGTCTCCTA TACCACAGGC
TCTGGGCATC ACCAGCGGCC CCAGGGAAAA AGAAAGAAAT
GGGAAACAGC ATGAAATCCA CCCCTGCGCC TGCCGAGAGG
CCCCTGCCCA ACCCGGAGGG ACTGGATAGC GACTTCCTTG
CCGTGCTAAG TGACTACCCG TCTCCTGACA TCAGCCCCCC
GATATTCCGC CGAGGGGAGA AACTGCGTGT GATTTCTGAT
GAAGGGGGCT GGTGGAAAGC TATTTCTCTT AGCACTGGTC
GAGAGAGTTA CATCCCTGGA ATATGTGTGG CCAGAGTTTA
CCATGGCTGG CTGTTTGAGG GCCTGGGCAG AGACAAGGCC
GAGGAGCTGC TGCAGCTGCC AGACACAAAG GTCGGCTCCT
TCATGATCAG AGAGAGTGAG ACCAAGAAAG GGTTTTACTC
ACTGTCGGTG AGACACAGGC AGGTAAAGCA TTACCGCATT
TTCCGTCTGC CCAACAACTG GTACTACATT TCCCCGAGGC
TCACCTTCCA GTGCCTGGAG GACCTGGTGA ACCACTATTC
TGAGGTGGCT GATGGCCTGT GCTGTGTGCT CACCACGCCC
TGCCTGACAC AAAGCACGGC TGCCCCAGCA GTGAGGGCCT
CCAGCTCACC TGTCACCTTG CGTCAGAAGA CTGTGGACTG
GAGGAGAGTG TCCAGACTGC AGGAGGACCC CGAGGGAACA
GAGAACCCGC TTGGGGTAGA CGAGTCCCTT TTCAGCTATG
GCCTTCGAGA GAGCATTGCC TCTTACCTGT CCCTGACCAG
TGAGGACAAC ACCTCCTTTG ATCGAAAGAA GAAAAGCATC
TCCCTGATGT ATGGTGGCAG CAAGAGAAAG AGCTCATTCT
TCTCATCACC ACCTTACTTT GAGGACTAGC CAAGAACAGA
CACAATGGTT CATGCCCAAA AGGAACAGAA GTTCCAACTA
TTGCCTGGGA TCTTGCGAAA AGCGAGGTTC CCTGATCCCT
GGGAGCCTCA CGTATTTTAG AAGCCAAGAG AAGCCACATG
GAGACTCAAA TTCGCATCTT CTCTATCCAC ATCATGACCA
AAGGAACCCC TCCCTGGTGT CTGATCAGGG CTGTGGCATC
ACGAAACATT GGATCATGAC ATGTCGGGCG ATGCTTGGAA
GAGCCCAGCA TGTATGTATG CACACATTGT GTGTGTGGGA
AGGACAAAGC CACTCTCACA AGAAAGGGCA CCAGGACTGC
TCTCCAAGGA ACTGGACCTG TCCAGACAGT TACACTCCAA
GGTCATTGGA GAGAACTTCT GTATGGGCAA GCCTGAGAGG
GAGAGGAAAC AAAAGCTGTG TCCTGGCAGA AGGTCTGGGT
TTGCAGATGG GTGCCCTGAA TGGAACTACT TTAACTAATC
CATAGGGACT TCTGGTATGC TTTCCTCTCT TTTTAAAGGA
ACTTCGTGAC ACTAAACATT AGCCCAAAGG ACTTCTTAGC
CTTCAATTGG GAGATACCTT TGGTCTGCTC CTGCACCAAA
GCCATATGGG TGGAAGTCAG TTGGCCTCCC TGGTTCTGCA
GAGGGCCAGA AGAATGAGAG AGAGGAAGAC TGCTGGCAGG
GAAATCGAGG AGGCGAGACT AGAACTGCAC CAGCTTCCCT
GATGTCTGCA GCCATGGCTT TGCAGCGCAG ACAGAGCTTC
TCTGGGATGC TGGGATTCTT GCCTGTATGA ATGCATCAAG
TATTCATTTA TTGCCCGAAT AGGCATTGCA TTAAGTCCTC
TGTAAGGTGT CAGGCAAGCC AAAAAAAAAA AAAAGATGCG
TAAGTCCTAA CCCCCAACAG AGGTGTTCAC AGTGTAGACA
GGGAAAAAAT GTATAAACAA ATGTGTAAAA AGAGAAATCA
GCTCATGGCT TAGGATGGAA TTAGAGACAG GTGAGGGACA
CTCAGGAGCT CATTTTCCAG CTGCTCTTCA GAGTGGAAGG
GCTGGCTGGA TCGGGTAGGT AAGAATAGCT GGATTTTTTA
GAAAAGAAAT GGATACAGTC TAAAGAATTA ACTCACCCGG
TACTTTATTC TAAGAAGGGT CTGGCATCCA TATGAGGAAA
AATGCTCAGC TCCAGGAAAG ATGGGGAGTC CAAGTGGATT
AATGATGTCA TGCATAATTT TAAGAGACAA GGGAGAAAAC
ACAATGTATA GCCAGAGAAG GAGAAGCTCC CATCCAAATC
CTACTAGGAA GAGAGTGGGC TGCAGATGAA TCTGTGACTC
ATGTTTCCCT GTTTCAAAGG GATCCTGGGG AAGGAGGGGA
ACATGCTTGC AGTATCTCTC CCTGTCTGTC TGCTCACATA
AGCATTCCGT CCATCTGAGC TCATCGTGCT ACTGGTATGT
GTATGTGCAG TTACACAGTT TTCTGTATCA TAGATTCTAG
TGTGTTTATA CAAGGAGACA TCTGTGGTTT CCCCAACCGT
TCCAAAAGGC TATTTCAAAG GAACCAGCCA ACGTATGAGA
AATGAATGTA ACACTGTGGA CATTGACTTC CCGCATAAGG
CAGGGTGACC CCCTGAACTC CAGATGTCTG CACAGTATCT
TATGTGTTGT TTTCCGTTGT GACGAATGTG ATTGGAACAT
TTGGGGAGCA CCCAGAGGGA TTTCTCAGTG GGAAGCATTA
CACTTTGCTA AATCATGTAT TTATTCCTGA TTAAAACAAA
CCTAATAAAT ATTTAACCCT TGGCAAAAAA AAAAAAAAAA
AAAAAAAAAA AAAAAAAAAA AAAAAAAAAA AA.

Human SLA has several isoforms. One isoform of human SLA has the following amino acid sequence:

(SEQ ID NO: 23)
MGNSMKSTPA PAERPLPNPE GLDSDFLAVL SDYPSPDISP
PIFRRGEKLR VISDEGGWWK AISLSTGRES YIPGICVARV
YHGWLFEGLG RDKAEELLQL PDTKVGSFMI RESETKKGFY
SLSVRHRQVK HYRIFRLPNN WYYISPRLTF QCLEDLVNHY
SEVADGLCCV LITPCLIQST AAPAVRASSS PVILRQKTVD
WRRVSRLQED PEGTENPLGV DESLFSYGLR ESIASYLSLT
SEDNTSFDRK KKSISLMYGG SKRKSSFFSS PPYFED

Another isoform of human SLA has the following amino acid sentience:

(SEQ ID NO: 24)
MLHRLWASPA APGKKKEMGN SMKSTPAPAE RPLPNPEGLD
SDFLAVLSDY PSPDISPPIF RRGEKLRVIS DEGGWWKAIS
LSTGRESYIP GICVARVYHG WLFEGLGRDK AEELLQLPDT
KVGSFMIRES ETKKGFYSLS VRHRQVKHYR IFRLPNNWYY
ISPRLTFQCL EDLVNHYSEV ADGLCCVLTT PCLTQSTAAP
AVRASSSPVT LRQKTVDWRR VSRLQEDPEG TENPLGVDES
LFSYGLRESI ASYLSLTSED NTSFDRKKKS ISLMYGGSKR
KSSFFSSPPY FED.

Still another isoform of human SLA has the following amino acid sequence:

(SEQ ID NO: 25)
MLSKLGHSPL GGLRARLTFP VCLLYHRLWA SPAAPGKKKE
MGNSMKSTPA PAERPLPNPE GLDSDFLAVL SDYPSPDISP
PIFRRGEKLR VISDEGGWWK AISLSTGRES YIPGICVARV
YHGWLFEGLG RDKAEELLQL PDTKVGSFMI RESETKKGFY
SLSVRHRQVK HYRIFRLPNN WYYISPRLTF QCLEDLVNHY
SEVADGLCCV LTTPCLTQST AAPAVRASSS PVTLRQKTVD
WRRVSRLQED PEGTENPLGV DESLFSYGLR ESIASYLSLT
SEDNTSFDRK KKSISLMYGG SKRKSSFFSS PPYFED.

Thrombomodulin (CD141, or BDCA-3) is an integral membrane protein expressed on the surface of endothelial cells. In humans, thrombomodulin is encoded by the THBD gene. Thrombomodulin functions as a cofactor in the thrombin-induced activation of protein C in the anticoagulant pathway by forming a 1:1 stoichiometric complex with thrombin. This raises the speed of protein C activation a thousandfold. Thrombomodulin-bound thrombin has no procoagulant effect. The TT-complex also inhibits fibrinolysis by cleaving thrombin-activatable fibrinolysis inhibitor (TAFI) into its active form. Human THBD has the following nucleic acid sequence:

(SEQ ID NO: 3)
GGCTGCCTCG CAGGGGCTGC GCGCAGCGGC AAGAAGTGTC
TGGGCTGGGA CGGACAGGAG AGGCTGTCGC CATCGGCGTC
CTGTGCCCCT CTGCTCCGGC ACGGCCCTGT CGCAGTGCCC
GCGCTTTCCC CGGCGCCTGC ACGCGGCGCG CCTGGGTAAC
ATGCTTGGGG TCCTGGTCCT TGGCGCGCTG GCCCTGGCCG
GCCTGGGGTT CCCCGCACCC GCAGAGCCGC AGCCGGGTGG
CAGCCAGTGC GTCGAGCACG ACTGCTTCGC GCTCTACCCG
GGCCCCGCGA CCTTCCTCAA TGCCAGTCAG ATCTGCGACG
GACTGCGGGG CCACCTAATG ACAGTGCGCT CCTCGGTGGC
TGCCGATGTC ATTTCCTTGC TACTGAACGG CGACGGCGGC
GTTGGCCGCC GGCGCCTCTG GATCGGCCTG CAGCTGCCAC
CCGGCTGCGG CGACCCCAAG CGCCTCGGGC CCCTGCGCGG
CTTCCAGTGG GTTACGGGAG ACAACAACAC CAGCTATAGC
AGGTGGGCAC GGCTCGACCT CAATGGGGCT CCCCTCTGCG
GCCCGTTGTG CGTCGCTGTC TCCGCTGCTG AGGCCACTGT
GCCCAGCGAG CCGATCTGGG AGGAGCAGCA GTGCGAAGTG
AAGGCCGATG GCTTCCTCTG CGAGTTCCAC TTCCCAGCCA
CCTGCAGGCC ACTGGCTGTG GAGCCCGGCG CCGCGGCTGC
CGCCGTCTCG ATCACCTACG GCACCCCGTT CGCGGCCCGC
GGAGCGGACT TCCAGGCGCT GCCGGTGGGC AGCTCCGCCG
CGGTGGCTCC CCTCGGCTTA CAGCTAATGT GCACCGCGCC
GCCCGGAGCG GTCCAGGGGC ACTGGGCCAG GGAGGCGCCG
GGCGCTTGGG ACTGCAGCGT GGAGAACGGC GGCTGCGAGC
ACGCGTGCAA TGCGATCCCT GGGGCTCCCC GCTGCCAGTG
CCCAGCCGGC GCCGCCCTGC AGGCAGACGG GCGCTCCTGC
ACCGCATCCG CGACGCAGTC CTGCAACGAC CTCTGCGAGC
ACTTCTGCGT TCCCAACCCC GACCAGCCGG GCTCCTACTC
GTGCATGTGC GAGACCGGCT ACCGGCTGGC GGCCGACCAA
CACCGGTGCG AGGACGTGGA TGACTGCATA CTGGAGCCCA
GTCCGTGTCC GCAGCGCTGT GTCAACACAC AGGGTGGCTT
CGAGTGCCAC TGCTACCCTA ACTACGACCT GGTGGACGGC
GAGTGTGTGG AGCCCGTGGA CCCGTGCTTC AGAGCCAACT
GCGAGTACCA GTGCCAGCCC CTGAACCAAA CTAGCTACCT
CTGCGTCTGC GCCGAGGGCT TCGCGCCCAT TCCCCACGAG
CCGCACAGGT GCCAGATGTT TTGCAACCAG ACTGCCTGTC
CAGCCGACTG CGACCCCAAC ACCCAGGCTA GCTGTGAGTG
CCCTGAAGGC TACATCCTGG ACGACGGTTT CATCTGCACG
GACATCGACG AGTGCGAAAA CGGCGGCTTC TGCTCCGGGG
TGTGCCACAA CCTCCCCGGT ACCTTCGAGT GCATCTGCGG
GCCCGACTCG GCCCTTGCCC GCCACATTGG CACCGACTGT
GACTCCGGCA AGGTGGACGG TGGCGACAGC GGCTCTGGCG
AGCCCCCGCC CAGCCCGACG CCCGGCTCCA CCTTGACTCC
TCCGGCCGTG GGGCTCGTGC ATTCGGGCTT GCTCATAGGC
ATCTCCATCG CGAGCCTGTG CCTGGTGGTG GCGCTTTTGG
CGCTCCTCTG CCACCTGCGC AAGAAGCAGG GCGCCGCCAG
GGCCAAGATG GAGTACAAGT GCGCGGCCCC TTCCAAGGAG
GTAGTGCTGC AGCACGTGCG GACCGAGCGG ACGCCGCAGA
GACTCTGAGC GGCCTCCGTC CAGGAGCCTG GCTCCGTCCA
GGAGCCTGTG CCTCCTCACC CCCAGCTTTG CTACCAAAGC
ACCTTAGCTG GCATTACAGC TGGAGAAGAC CCTCCCCGCA
CCCCCCAAGC TGTTTTCTTC TATTCCATGG CTAACTGGCG
AGGGGGTGAT TAGAGGGAGG AGAATGAGCC TCGGCCTCTT
CCGTGACGTC ACTGGACCAC TGGGCAATGA TGGCAATTTT
GTAACGAAGA CACAGACTGC GATTTGTCCC AGGTCCTCAC
TACCGGGCGC AGGAGGGTGA GCGTTATTGG TCGGCAGCCT
TCTGGGCAGA CCTTGACCTC GTGGGCTAGG GATGACTAAA
ATATTTATTT TTTTTAAGTA TTTAGGTTTT TGTTTGTTTC
CTTTGTTCTT ACCTGTATGT CTCCAGTATC CACTTTGCAC
AGCTCTCCGG TCTCTCTCTC TCTACAAACT CCCACTTGTC
ATGTGACAGG TAAACTATCT TGGTGAATTT TTTTTTCCTA
GCCCTCTCAC ATTTATGAAG CAAGCCCCAC TTATTCCCCA
TTCTTCCTAG TTTTCTCCTC CCAGGAACTG GGCCAACTCA
CCTGAGTCAC CCTACCTGTG CCTGACCCTA CTTCTTTTGC
TCTTAGCTGT CTGCTCAGAC AGAACCCCTA CATGAAACAG
AAACAAAAAC ACTAAAAATA AAAATGGCCA TTTGCTTTTT
CACCAGATTT GCTAATTTAT CCTGAAATTT CAGATTCCCA
GAGCAAAATA ATTTTAAACA AAGGTTGAGA TGTAAAAGGT
ATTAAATTGA TGTTGCTGGA CTGTCATAGA AATTACACCC
AAAGAGGTAT TTATCTTTAC TTTTAAACAG TGAGCCTGAA
TTTTGTTGCT GTTTTGATTT GTACTGAAAA ATGGTAATTG
TTGCTAATCT TCTTATGCAA TTTCCTTTTT TGTTATTATT
ACTTATTTTT GACAGTGTTG AAAATGTTCA GAAGGTTGCT
CTAGATTGAG AGAAGAGACA AACACCTCCC AGGAGACAGT
TCAAGAAAGC TTCAAACTGC ATGATTCATG CCAATTAGCA
ATTGACTGTC ACTGTTCCTT GTCACTGGTA GACCAAAATA
AAACCAGCTC TACTGGTCTT GTGGAATTGG GAGCTTGGGA
ATGGATCCTG GAGGATGCCC AATTAGGGCC TAGCCTTAAT
CAGGTCCTCA GAGAATTTCT ACCATTTCAG AGAGGCCTTT
TGGAATGTGG CCCCTGAACA AGAATTGGAA GCTGCCCTGC
CCATGGGAGC TGGTTAGAAA TGCAGAATCC TAGGCTCCAC
CCCATCCAGT TCATGAGAAT CTATATTTAA CAAGATCTGC
AGGGGGTGTG TCTGCTCAGT AATTTGAGGA CAACCATTCC
AGACTGCTTC CAATTTTCTG GAATACATGA AATATAGATC
AGTTATAAGT AGCAGGCCAA GTCAGGCCCT TATTTTCAAG
AATTTGAGGA ATTTTCTTTG TGTAGCTTTG CTCTTTGGTA
GAAAAGGCTA GGTACACAGC TCTAGACACT GCCACACAGG
GTCTGCAAGG TCTTTGGTTC AGCTAAGCTA GGAATGAAAT
CCTGCTTCAG TGTATGGAAA TAAATGTATC ATAGAAATGT
AACTTTTGTA AGACAAAGGT TTTCCTCTTC TATTTTGTAA
ACTCAAAATA TTTGTACATA GTTATTTATT TATTGGAGAT
AATCTAGAAC ACAGGCAAAA TCCTTGCTTA TGACATCACT
TGTACAAAAT AAACAAATAA CAATGTGCTC TCGGGTTGTG
TGTCTGTTCA CTTTTCCTCC CTCAGTGCCC TCATTTTATG
TCATTAAATG GGGCTCACAA ACCATGCAAA TGCTATGAGA
TGCATGGAGG GCTGCCCTGT ACCCCAGCAC TTGTGTTGTC
TGGTGGTGGC ACCATCTCTG ATTTTCAAAG CTTTTTCCAG
AGGCTATTAT TTTCACTGTA GAATGATTTC ATGCTATCTC
TGTGTGCACA AATATTTATT TTCTTTCTGT AACCATAACA
ACTTCATATA TGAGGACTTG TGTCTCTGTG CTTTTAAATG
CATAAATGCA TTATAGGATC ATTTGTTGGA ATGAATTAAA
TAAACCCTTC CTGGGGCATC TGGCGAATCC CAAAAAAAAA
AAAAAAAA

Human Thrombomodulin has the following amino acid sequence:

(SEQ ID NO: 26)
MLGVLVLGAL ALAGLGFPAP AEPQPGGSQC VEHDCFALYP
GPATFLNASQ ICDGLRGHLM TVRSSVAADV ISLLLNGDGG
VGRRRLWIGL QLPPGCGDPK RLGPLRGFQW VTGDNNTSYS
RWARLDLNGA PLCGPLCVAV SAAEATVPSE PIWEEQQCEV
KADGFLCEFH FPATCRPLAV EPGAAAAAVS ITYGTPFAAR
GADFQALPVG SSAAVAPLGL QLMCTAPPGA VQGHWAREAP
GAWDCSVENG GCEHACNAIP GAPRCQCPAG AALQADGRSC
TASATQSCND LCEHFCVPNP DQPGSYSCMC ETGYRLAADQ
HRCEDVDDCI LEPSPCPQRC VNTQGGFECH CYPNYDLVDG
ECVEPVDPCF RANCEYQCQP LNQTSYLCVC AEGFAPIPHE
PHRCQMFCNQ TACPADCDPN TQASCECPEG YILDDGFICT
DIDECENGGF CSGVCHNLPG TFECICGPDS ALARHIGTDC
DSGKVDGGDS GSGEPPPSPT PGSTLTPPAV GLVHSGLLIG
ISIASLCLVV ALLALLCHLR KKQGAARAKM EYKCAAPSKE
VVLQHVRTER TPQRL

Runt-related transcription factor 2 (RUNX2) is a protein that in humans is encoded by the RUNX2 gene. RUNX2 is a member of the RUNX family of transcription factors and has a Runt DNA-binding domain. It is essential for osteoblastic differentiation and skeletal morphogenesis and acts as a scaffold for nucleic acids and regulatory factors involved in skeletal gene expression. The protein can bind DNA both as a monomer or, with more affinity, as a subunit of a heterodimeric complex. Transcript variants of the gene that encode different protein isoforms result from the use of alternate promoters as well as alternate splicing. Human RUNX2 has the following nucleic acid sequence:

(SEQ ID NO: 4)
GTGTGAATGC TTCATTCGCC TCACAAACAA CCACAGAACC
ACAAGTGCGG TGCAAACTTT CTCCAGGAGG ACAGCAAGAA
GTCTCTGGTT TTTAAATGGT TAATCTCCGC AGGTCACTAC
CAGCCACCGA GACCAACAGA GTCATTTAAG GCTGCAAGCA
GTATTTACAA CAGAGGGTAC AAGTTCTATC TGAAAAAAAA
AGGAGGGACT ATGGCATCAA ACAGCCTCTT CAGCACAGTG
ACACCATGTC AGCAAAACTT CTTTTGGGAT CCGAGCACCA
GCCGGCGCTT CAGCCCCCCC TCCAGCAGCC TGCAGCCCGG
CAAAATGAGC GACGTGAGCC CGGTGGTGGC TGCGCAACAG
CAGCAGCAAC AGCAGCAGCA GCAGCAGCAG CAGCAGCAGC
AGCAACAGCA GCAGCAGCAG CAGGAGGCGG CGGCGGCGGC
TGCGGCGGCG GCGGCGGCTG CGGCGGCGGC AGCTGCAGTG
CCCCGGTTGC GGCCGCCCCA CGACAACCGC ACCATGGTGG
AGATCATCGC CGACCACCCG GCCGAACTCG TCCGCACCGA
CAGCCCCAAC TTCCTGTGCT CGGTGCTGCC CTCGCACTGG
CGCTGCAACA AGACCCTGCC CGTGGCCTTC AAGGTGGTAG
CCCTCGGAGA GGTACCAGAT GGGACTGTGG TTACTGTCAT
GGCGGGTAAC GATGAAAATT ATTCTGCTGA GCTCCGGAAT
GCCTCTGCTG TTATGAAAAA CCAAGTAGCA AGGTTCAACG
ATCTGAGATT TGTGGGCCGG AGTGGACGAG GCAAGAGTTT
CACCTTGACC ATAACCGTCT TCACAAATCC TCCCCAAGTA
GCTACCTATC ACAGAGCAAT TAAAGTTACA GTAGATGGAC
CTCGGGAACC CAGAAGGCAC AGACAGAAGC TTGATGACTC
TAAACCTAGT TTGTTCTCTG ACCGCCTCAG TGATTTAGGG
CGCATTCCTC ATCCCAGTAT GAGAGTAGGT GTCCCGCCTC
AGAACCCACG GCCCTCCCTG AACTCTGCAC CAAGTCCTTT
TAATCCACAA GGACAGAGTC AGATTACAGA CCCCAGGCAG
GCACAGTCTT CCCCGCCGTG GTCCTATGAC CAGTCTTACC
CCTCCTACCT GAGCCAGATG ACGTCCCCGT CCATCCACTC
TACCACCCCG CTGTCTTCCA CACGGGGCAC TGGGCTTCCT
GCCATCACCG ATGTGCCTAG GCGCATTTCA GGTGCTTCAG
AACTGGGCCC TTTTTCAGAC CCCAGGCAGT TCCCAAGCAT
TTCATCCCTC ACTGAGAGCC GCTTCTCCAA CCCACGAATG
CACTATCCAG CCACCTTTAC TTACACCCCG CCAGTCACCT
CAGGCATGTC CCTCGGTATG TCCGCCACCA CTCACTACCA
CACCTACCTG CCACCACCCT ACCCCGGCTC TTCCCAAAGC
CAGAGTGGAC CCTTCCAGAC CAGCAGCACT CCATATCTCT
ACTATGGCAC TTCGTCAGGA TCCTATCAGT TTCCCATGGT
GCCGGGGGGA GACCGGTCTC CTTCCAGAAT GCTTCCGCCA
TGCACCACCA CCTCGAATGG CAGCACGCTA TTAAATCCAA
ATTTGCCTAA CCAGAATGAT GGTGTTGACG CTGATGGAAG
CCACAGCAGT TCCCCAACTG TTTTGAATTC TAGTGGCAGA
ATGGATGAAT CTGTTTGGCG ACCATATTGA AATTCCTCAG
CAGTGGCCCA GTGGTATCTG GGGGCCACAT CCCACACGTA
TCAATATATA CATATATAGA GAGAGTGCAT ATATATGTAT
ATCGATTAGC TATCTACAAA GTGCCTATTT TTTAGAAGAT
TTTTCATTCA CTCACTCAGT CATGATCTTG CAGCCATAAG
AGGGTAGATA TTGAGAAGCA GAAGGCTCAA GAGAGACAAT
TGCAATCGAG CTTCAGATTG TTTACTATTT AAGATGTACT
TTTACAAAGG AACAAAGAAG GGAAAAGGTA TTTTTGTTTT
TGTTGTTTGG TCTGTTATCA TCAATAACCT GTTCATATGC
CAATTCAGAG AGGTGGACTC CAGGTTCAGG AGGGAGAAGA
GCAAAGCCGC TTCCTCTCTG TGCTTTGAAA CTTCACACCC
TCACGGTGGC AGCTGTGTAT GGACCAGTGC CCTCCGCAGA
CAGCTCACAA AACCAGTTGA GGTGCACTAA AGGGACATGA
GGTAGAATGG ATGCTTCCAT CACAGTACCA TCATTCAGAA
TAACTCTTCC AATTTCTGCT TTCAGACATG CTGCAGGTCC
TCATCTGAAC TGTTGGGTTC GTTTTTTTTT TTTTTTTTCC
TGCTCCAAGA AAGTGACTTC AAAAATAACT GATCAGGATA
GATTATTTTA TTTTACTTTT TAACACTCCT TCTCCCCTTT
TCCCACTGAA CCAAAAAGAA ATCCCATCCC TAAAACCTGC
CTTCTCCTTT TATGCAAAAC TGAAAATGGC AATACATTAT
TATAGCCATA ATGGTATAGA TAGTGATTGC GTTTGGCTAT
GTGTTGTTTT CTTTTTTTTT AAATTATGAA TATGTGTAAA
ATCTGAGGTA ACTTGCTAAC GTGAATGGTC ATATAACTTT
AAAGATATAT TTATAATTAT TTAATGACAT TTGGACCCTT
GAAACATTTC TTAGTGTATT GATATGTTGA CTTCGGTCTC
TAAAAGTGCT CTTTATTAAA TAACAAATTT CTTCAGTGGT
CTAGAGCCAT ATCTGAAATA TTGCTAAGCA ATTTCAGTTC
ATCCAGGCAC AATGTGATTT TAAAAAATAC TTCCATCTCC
AAATATTTTA GATATAGATT GTTTTTGTGA TGTATGAAGG
AAATGTTATG TTTAGTTCTT TCAGATCTTT GAATGCCTCT
AACACAGCTT TGCCTTCTAA AGCGGTAATT AGGGATTTAA
AAAACAACCT TTAGCCCTTT ATCAGCATGA AATGCTGGAG
TGATGTGGTT TTCTAATTTC TTTGGGGTAA TTATGACTCT
TGTCATATTA AAAAGACAAG CACAAGTAAA TCATTGAACT
ACAGAAAAAT GTTCTGTGGT TTCATAGTTA AGCAAAACTC
TAAATCGCCA GGCTTCATAG CAAAGACATA GTCAGCTAAA
AGCCGCACAT GTGGATAGAG GGTTCAATTA TGAGACACCT
AGTACAGGAG AGCAAAATTG CACCAGAGAT TCTTAACCAA
CCAGCCTTAC CAAACAACAC AACAGGGGAA CCCCAATCTG
CCTTACCCAA GGCCCCACTG GCAGCTTTCC ACAGAATTTG
CATTTAGAGG AGCAGAATGA CATCACTGTC CTTTGGGAGT
AGGTCCTCTG AAAAGGCAGC AGGTTCCAGC AGGTAGCTGA
GCTGAGAGGA CATATGGCCC ACGGGGACCT ACAGACAGCC
TTTGACATTT GTATTTCTTA CAATGGAGGG CCAAGGAGGG
CAAGGGGCTG TGGAGTTTGG TGTCTACTAG TGTGTATGAA
TTTGAGCTAG AGTCCTTCTG TGGCATGCAC TTTGACCACT
CCTGGCAGTC ACATGGCAGA TTTCCAAGTG CAAATCCTTA
ATCCAAACAA GGATCATCTA ATGACACCAC CAGGCCAATC
CCTGCTCTCC TCCCCGAAAA GTCAGGGTCC CTTCATTGGA
ATCCTCCACC CACCCAAGCA GAATTTAGCA GAGATTTGCC
TTCAAACCCT AACGGCCCCC TTGTTCTCTG GTCCTTCTCA
AACCCACCTT TGTAGGCCAC CCAGCATTGC AGGACAGCGT
GTGGGGCAGC TGGACCTGTG CTTCCTGCCT GGGAGTCTCC
CTTGGAATTC ATCCTGACTC CTTCTAATAA AAATGGATGG
GAAAGCAAAA CACTTTGCCT TCTAAAGGCC GTATACCAAG
TATGCTTAGA TAAATAAGCC ACTTTTCTAT TACTTAAGTA
AGAAGGAAGT AGTAATTGAT ACTATTTATT GTTTGTGTGT
GGTAGCTTGA AGCACACCAC TGTCCATTTA TTTGTAAGTG
TAAAATATGT GTGTTTGTTT CAGCAGCACT TAAAAAAGCC
AGTGTCTGGT TACACATTTC AATTTTAATT AATTGACATA
AAAATGCTAC CGCCAGTGCC AGCTGCATCC TATTTAATTA
AAAAGGTACT ATATTTGTAC ATTATTTTTT AATGTTAAAA
GGGCTTTTTT AAGTTTACAG TACACATACC GAGTGACTTT
AGGGATGCTT TTGTGTTGAA ATGTTACTAT AGTGGCTGCA
GGCAGCAACC CAGAAACACT TTAGAAGCTT TTTTTCCTTG
GGAAAAATTC AAGCACTTCT TCCCTCCACC CTCACTCCAA
CCACCCCAAT GGGGGTAATT CACATTTCTT AGAACAAATT
CTGCCCTTTT TTGGTCTAGG GATTAAAATT TTGTTTTTCT
TTCTTTCTTT TTTTTTTTTT TTCACTGAAC CCTTAATTTG
CACTGGGTCA TGTGTTTGAT TTGTGATTTC AAGACCAAAG
CAAAGTCTTA CTACTACTGT GGAACCATGT ACTAGTTCCT
GGGAATTAAA ATAGCGTGGT TCTCTTTGTA GCACAAACAT
TGCTGGAATT TGCAGTCTTT TCAATGCAGC CACATTTTTA
TCCATTTCAG TTGTCTCACA AATTTTAACC CATATCAGAG
TTCCAGAACA GGTACCACAG CTTTGGTTTT AGATTAGTGG
AATAACATTC AGCCCAGAAC TGAGAAACTC AACAGATTAA
CTATCGTTTG CTCTTTAGAC GGTCTCACTG CCTCTCACTT
GCCAGAGCCC TTTCAAAATG AGCAGAGAAG TCCACACCAT
TAGGGACCAT CTGTGATAAA TTCAGAAGGG AGGAGATGTG
TGTACAGCTT TAAGGATTCC CTCAATTCCG AGGAAAGGGA
CTGGCCCAGA ATCCAGGTTA ATACATGGAA ACACGAAGCA
TTAGCAAAAG TAATAATTAT ACCTATGGTA TTTGAAAGAA
CAATAATAAA AGACACTTCT TCCAAACCTT GAATTTGTTG
TTTTTAGAAA ACGAATGCAT TTAAAAATAT TTTCTATGTG
AGAATTTTTT AGATGTGTGT TTACTTCATG TTTACAAATA
ACTGTTTGCT TTTTAATGCA GTACTTTGAA ATATATCAGC
CAAAACCATA ACTTACAATA ATTTCTTAGG TATTCTGAAT
AAAATTCCAT TTCTTTTGGA TATGCTTTAC CATTCTTAGG
TTTCTGTGGA ACAAAAATAT TTGTAGCATT TTGTGTAAAT
ACAAGCTTTC ATTTTTATTT TTTCCAATTG CTATTGCCCA
AGAATTGCTT TCCATGCACA TATTGTAAAA ATTCCGCTTT
GTGCCACAGG TCATGATTGT GGATGAGTTT ACTCTTAACT
TCAAAGGGAC TATTTGTATT GTATGTTGCA ACTGTAAATT
GAATTATTTG GCATTTTTCT CATGATTGTA ATATTAATTT
GAAGTTTGAA TTTAATTTTC AATAAAATGG CTTTTTTGGT
TTTGTTA

Human RUNX2 has several isoforms. One isoform of human RUNX2 has the following amino acid sequence:

(SEQ ID NO: 27)
MASNSLFSTV TPCQQNFFWD PSTSRRFSPP SSSLQPGKMS
DVSPVVAAQQ QQQQQQQQQQ QQQQQQQQQQ QEAAAAAAAA
AAAAAAAAAV PRLRPPHDNR TMVEIIADHP AELVRTDSPN
FLCSVLPSHW RCNKTLPVAF KVVALGEVPD GTVVTVMAGN
DENYSAELRN ASAVMKNQVA RFNDLRFVGR SGRGKSFTLT
ITVFTNPPQV ATYHRAIKVT VDGPREPRRH RQKLDDSKPS
LFSDRLSDLG RIPHPSMRVG VPPQNPRPSL NSAPSPFNPQ
GQSQITDPRQ AQSSPPWSYD QSYPSYLSQM TSPSIHSTTP
LSSTRGTGLP AITDVPRRIS GASELGPFSD PRQFPSISSL
TESRFSNPRM HYPATFTYTP PVTSGMSLGM SATTHYHTYL
PPPYPGSSQS QSGPFQTSST PYLYYGTSSG SYQFPMVPGG
DRSPSRMLPP CTTTSNGSTL LNPNLPNQND GVDADGSHSS
SPTVLNSSGR MDESVWRPY

Another isoform of human RUNX2 has the following amino acid sequence:

(SEQ ID NO: 28)
MASNSLFSTV TPCQQNFFWD PSTSRRFSPP SSSLQPGKMS
DVSPVVAAQQ QQQQQQQQQQ QQQQQQQQQQ QEAAAAAAAA
AAAAAAAAAV PRLRPPHDNR TMVEIIADHP AELVRTDSPN
FLCSVLPSHW RCNKTLPVAF KVVALGEVPD GTVVTVMAGN
DENYSAELRN ASAVMKNQVA RFNDLRFVGR SGRGKSFTLT
ITVFTNPPQV ATYHRAIKVT VDGPREPRRH RQKLDDSKPS
LFSDRLSDLG RIPHPSMRVG VPPQNPRPSL NSAPSPFNPQ
GQSQITDPRQ AQSSPPWSYD QSYPSYLSQM TSPSIHSTTP
LSSTRGTGLP AITDVPRRIS DDDTATSDFC LWPSTLSKKS
QAGASELGPF SDPRQFPSIS SLTESRFSNP RMHYPATFTY
TPPVTSGMSL GMSATTHYHT YLPPPYPGSS QSQSGPFQTS
STPYLYYGTS SGSYQFPMVP GGDRSPSRML PPCTTTSNGS
TLLNPNLPNQ NDGVDADGSH SSSPTVLNSS GRMDESVWRP Y

Still another isoform of human RUNX2 has the following amino acid sequence:

(SEQ ID NO: 29)
MRIPVDPSTS RRFSPPSSSL QPGKMSDVSP VVAAQQQQQQ
QQQQQQQQQQ QQQQQQQEAA AAAAAAAAAA AAAAAVPRLR
PPHDNRTMVE IIADHPAELV RTDSPNFLCS VLPSHWRCNK
TLPVAFKVVA LGEVPDGTVV TVMAGNDENY SAELRNASAV
MKNQVARFND LRFVGRSGRG KSFTLTITVF TNPPQVATYH
RAIKVTVDGP REPRRHRQKL DDSKPSLFSD RLSDLGRIPH
PSMRVGVPPQ NPRPSLNSAP SPFNPQGQSQ ITDPRQAQSS
PPWSYDQSYP SYLSQMTSPS IHSTTPLSST RGTGLPAITD
VPRRISDDDT ATSDFCLWPS TLSKKSQAGA SELGPFSDPR
QFPSISSLTE SRFSNPRMHY PATFTYTPPV TSGMSLGMSA
TTHYHTYLPP PYPGSSQSQS GPFQTSSTPY LYYGTSSGSY
QFPMVPGGDR SPSRMLPPCT TTSNGSTLLN PNLPNQNDGV
DADGSHSSSP TVLNSSGRMD ESVWRPY

Runt-related transcription factor 3 (RUNX3) is a protein that in humans is encoded by the RUNX3 gene. RUNX3 is a member of the runt domain-containing family of transcription factors. A heterodimer of this protein and a beta subunit forms a complex that binds to a DNA sequence found in a number of enhancers and promoters, and can either activate or suppress transcription. It also interacts with other transcription factors. It functions as a tumor suppressor, and the gene is frequently deleted or transcriptionally silenced in cancer. Multiple transcript variants encoding different isoforms have been found for this gene. Human RUNX3 has the following amino nucleic sequence:

(SEQ ID NO: 5)
CCCGCCACTT GATTCTGGAG GATTTGTTCT GGGGCTGCGG
CCGCGGAGTC GGGGCGGCCG CGGGCGAGCT TCGGGGCGGG
AGGCGGCGGC AGCGGCACAG CCCCGCGCGG GCCCCGCCGC
GGCCCAGGCA GCCGGGACAG CCACGAGGGG CGGCCGCACG
CGGGGCCGCG CGCCGAGGAT GCGGGACTAG CCGGGCAGGC
TGCGGGCGGC CGTCGGGCCA GCGAGGCCTC GCAGCGGGCG
GGCCCTGGCG AGTAGTGGCC GGGCGCCGCC CCCTGCGCCC
TGAGGCCCGG GCCCCGCCGC TTCTGCTTTC CCGCTTCTCG
CGGCAGCGGC GGCCGAGGAG GCGCCCGCGC CGGCCGCCCC
CGGGGGAAGC CGCGCCGTCT CCGCCTGCCC GGCGCCCTGA
CGGCCGCTGT TATGCGTATT CCCGTAGACC CAAGCACCAG
CCGCCGCTTC ACACCTCCCT CCCCGGCCTT CCCCTGCGGC
GGCGGCGGCG GCAAGATGGG CGAGAACAGC GGCGCGCTGA
GCGCGCAGGC GGCCCTGGCG CCCGGAGGGC GCGCCCGGCC
CGAGGTGCGC TCGATGGTGG ACGTGCTGGC GGACCACGCA
GGCGAGCTCG TGCGCACCGA CAGCCCCAAC TTCCTCTGCT
CCGTGCTGCC CTCGCACTGG CGCTGCAACA AGACGCTGCC
CGTCGCCTTC AAGGTGGTGG CATTGGGGGA CGTGCCGGAT
GGTACGGTGG TGACTGTGAT GGCAGGCAAT GACGAGAACT
ACTCCGCTGA GCTGCGCAAT GCCTCGGCCG TCATGAAGAA
CCAGGTGGCC AGGTTCAACG ACCTTCGCTT CGTGGGCCGC
AGTGGGCGAG GGAAGAGTTT CACCCTGACC ATCACTGTGT
TCACCAACCC CACCCAAGTG GCGACCTACC ACCGAGCCAT
CAAGGTGACC GTGGACGGAC CCCGGGAGCC CAGACGGCAC
CGGCAGAAGC TGGAGGACCA GACCAAGCCG TTCCCTGACC
GCTTTGGGGA CCTGGAACGG CTGCGCATGC GGGTGACACC
GAGCACACCC AGCCCCCGAG GCTCACTCAG CACCACAAGC
CACTTCAGCA GCCAGCCCCA GACCCCAATC CAAGGCACCT
CGGAACTGAA CCCATTCTCC GACCCCCGCC AGTTTGACCG
CTCCTTCCCC ACGCTGCCAA CCCTCACGGA GAGCCGCTTC
CCAGACCCCA GGATGCATTA TCCCGGGGCC ATGTCAGCTG
CCTTCCCCTA CAGCGCCACG CCCTCGGGCA CGAGCATCAG
CAGCCTCAGC GTGGCGGGCA TGCCGGCCAC CAGCCGCTTC
CACCATACCT ACCTCCCGCC ACCCTACCCG GGGGCCCCGC
AGAACCAGAG CGGGCCCTTC CAGGCCAACC CGTCCCCCTA
CCACCTCTAC TACGGGACAT CCTCTGGCTC CTACCAGTTC
TCCATGGTGG CCGGCAGCAG CAGTGGGGGC GACCGCTCAC
CTACCCGCAT GCTGGCCTCT TGCACCAGCA GCGCTGCCTC
TGTCGCCGCC GGCAACCTCA TGAACCCCAG CCTGGGCGGC
CAGAGTGATG GCGTGGAGGC CGACGGCAGC CACAGCAACT
CACCCACGGC CCTGAGCACG CCAGGCCGCA TGGATGAGGC
CGTGTGGCGG CCCTACTGAC CGCCCTGGTG GACTCCTCCC
GCTGGAGGCG GGGACCCTAA CAACCTTCAA GACCAGTGAT
GGGCCGGCTC CGAGGCTCCG GGCGGGAATG GGACCTGCGC
TCCAGGGTGG TCTCGGTCCC AGGGTGGTCC CAGCTGGTGG
GAGCCTCTGG CTGCATCTGT GCAGCCACAT CCTTGTACAG
AGGCATAGGT TACCACCCCC ACCCCGGCCC GGGATACTGC
CCCCGGCCCA GATCCTGGCC GTCTCATCCC ATACTTCTGT
GGGGAATCAG CCTCCTGCCA CCCCCCCGGA AGGACCTCAC
TGTCTCCAGC TATGCCCAGT GCTGCATGGG ACCCATGTCT
CCTGGGACAG AGGCCATCTC TCTTCCAGAG AGAGGCAGCA
TTGGCCCACA GGATAAGCCT CAGGCCCTGG GAAACCTCCC
GACCCCTGCA CCTTCGTTGG AGCCCCTGCA TCCCCTGGGT
CCAGCCCCCT CTGCATTTAC ACAGATTTGA GTCAGAACTG
GAAAGTGTCC CCCACCCCCA CCACCCTCGA GCGGGGTTCC
CCTCATTGTA CAGATGGGGC AGGACCCAGC ACGCTGCTGG
CAGAGATGGT TTGAGAACAC ATCCAAGCCA GTCCCCCCAG
CCCAGCTTCC CCTCCGTTCC TAACTGTTGG CTTTCCCCCA
GCCGCACGGG TCCCAGGCCC CAGAGAAGAT GAGTCTATGG
CATCAGGTTC TTAAACCCAG GAAAGCACCT ACAGACCGGC
TCCTCCATGC ACTTTACCAG CTCAACGCAT CCACTCTCTG
TTCTCTTGGC AGGGCGGGGG AGGGGGGATA GGAGGTCCCC
TTTCCCCTAG GTGGTCTCAT AATTCCATTT GTGGAGAGAA
CAGGAGGGCC AGATAGATAG GTCCTAGCAG AAGGCATTGA
GGTGAGGGAT CATTTTGGGT CAGACATCAA TGTCCCTGTC
CCCCCTGGGT CCAGCCAAGC TGTGCCCCAT CCCCCAAGCC
TCCAGGGTGG ATCCAGCCAA ATCTTGCGAC TCCTGGCACA
CACCTGTCTG TAACCTGTTT TGTGCTCTGA AAGCAAATAG
TCCTGAGCAA AAAAAAAAAA AAAACAAAAA AACAAAAAAA
AAACAAAACA GTTTTTAAAA CTGATTTTAG AAAAAGAAGC
TTAATCTAAC GTTTTCAAAC ACAAGGTCTC TTACAGGTAT
AGTTCCGTGA TTATGATAGC TCTGTGATTA TAAGCAACAT
CCCCGCCCCC TCTCCCCCCC GCGGACCCCC AGCTGCCTCC
TGAGGGTGTG GGGTTATTAG GGTCTCAATA CTTTCTCAAG
GGGCTACACT CCCCATCAGG CAGCATCCCA CCAGCCTGCA
CCACAGGCTC CCCTGGGAGG ACGAGGGAAA CGCTGATGAG
ACGCTGGGCA TCTCTCCTCT GTGGCTCTAG GACATCTGTC
CAGGAGGCTG GGCGGAGGTG GGCAGGATGT GAGAGGTGGG
GAGTACTGGC TGTGCGTGGC AGGACAGAAG CACTGTAAAG
GGCTCTCCAG CCGCAGCTCA GCTGCACTGC GTTCCGAGGT
GAAGTCTTGC CCCTGAATTT TGCAAAATGG GAAAGTGGGC
GCTTGCCCAA GGGCCAGGCT GCATGGATTC TCACATCAGA
GTTCTCTGGC CCTAGAAAGG CTTAGAAAAG GCGTAAGGGA
ACTCATAAAG GCTAGCAGCA TGCGGTATTT TAACTTTCTG
CCTCGGCCTC TGTGGATGCA GAAATCTGCC CTACAAAATG
CTCTTCATTG GTTGTCTCTG TGAGAGCACT GTCCCCACCC
AACCTGTCAC AACGGCCAGA ACCATACACC AGAGACACAC
TGGCAGGTTA GGCAGTCCTT CTGGTGATCC TATTCCATTC
CCTCCTGCTG CGGTTTCTCT TGGCCTGTCC TCACTGGAAA
AACAGTCTCC ATCTCCTCAA AATAGTTGCT GACTCCCTGC
ACCCAAGGGG CCTCTCCATG CCTTCGTTGG AAGCAGCTAT
GAATCCATTG TCCTTGTAGT TTCTTCCCTC CTGTTCTCTG
GTTATAGCTG GTCCCAGGTC AGCGTGGGAG GCACCTTTGG
GTTCCCAGTG CCCAGCACTT TGTAGTCTCA TCCCAGATTA
CTAACCCTTC CTGATCCTGG AGAGGCAGGG ATAGTAAATA
AATTGCTCTT CCTACCCCAT CCCCCATCCC CTGACAAAAA
GTGACGGCAG CCGTACTGAG TCTGTAAGGC CCAAAGTGGG
TACAGACAGC CTGGGCTGGT AAAAGTAGGT CCTTATTTAC
AAGGCTGCGT TAAAGTTGTA CTAGGCAAAC ACACTGATGT
AGGAAGCACG AGGAAAGGAA GACGTTTTGA TATAGTGTTA
CTGTGAGCCT GTCAGTAGTG GGTACCAATC TTTTGTGACA
TATTGTCATG CTGAGGTGTG ACACCTGCTG CACTCATCTG
ATGTAAAACC ATCCCAGAGC TGGCGAGAGG ATGGAGCTGG
GTGGAAACTG CTTTGCACTA TCGTTTGCTT GGTGTTTGTT
TTTAACGCAC AACTTGCTTG TACAGTAAAC TGTCTTCTGT
ACTATTTAAC TGTAAAATGG AATTTTGACT GATTTGTTAC
AATAATATAA CTCTGAGATG TGTGGAAGGA

Human RUNX3 has at least two isoforms. One isoform of human RUNX3 has the following amino acid sequence:

(SEQ ID NO: 30)
MASNSIFDSF PTYSPTFIRD PSTSRRFTPP SPAFPCGGGG
GKMGENSGAL SAQAAVGPGG RARPEVRSMV DVLADHAGEL
VRTDSPNFLC SVLPSHWRCN KTLPVAFKVV ALGDVPDGTV
VTVMAGNDEN YSAELRNASA VMKNQVARFN DLRFVGRSGR
GKSFTLTITV FTNPTQVATY HRAIKVTVDG PREPRRHRQK
LEDQTKPFPD RFGDLERLRM RVTPSTPSPR GSLSTTSHFS
SQPQTPIQGT SELNPFSDPR QFDRSFPTLP TLTESRFPDP
RMHYPGAMSA AFPYSATPSG TSISSLSVAG MPATSRFHHT
YLPPPYPGAP QNQSGPFQAN PSPYHLYYGT SSGSYQFSMV
AGSSSGGDRS PTRMLASCTS SAASVAAGNL MNPSLGGQSD
GVEADGSHSN SPTALSTPGR MDEAVWRPY

Another isoform of human RUNX3 has the following amino acid sequence:

(SEQ ID NO: 31)
MRIPVDPSTS RRFTPPSPAF PCGGGGGKMG ENSGALSAQA
AVGPGGRARP EVRSMVDVLA DHAGELVRTD SPNFLCSVLP
SHWRCNKTLP VAFKVVALGD VPDGTVVTVM AGNDENYSAE
LRNASAVMKN QVARFNDLRF VGRSGRGKSF TLTITVFTNP
TQVATYHRAI KVTVDGPREP RRHRQKLEDQ TKPFPDRFGD
LERLRMRVTP STPSPRGSLS TTSHFSSQPQ TPIQGTSELN
PFSDPRQFDR SFPTLPTLTE SREPDPRMHY PGAMSAAFPY
SATPSGTSTS SLSVAGMPAT SRFHHTYLPP PYPGAPQNQS
GPFQANPSPY HLYYGTSSGS YQFSMVAGSS SGGDRSPTRM
LASCTSSAAS VAAGNLMNPS LGGQSDGVEA DGSHSNSPTA
LSTPGRMDEA VWRPY

Protocadherin 17 (PCDH17) is a protein that in humans is encoded by the PCDH17 gene. This gene belongs to the protocadherin gene family, a subfamily of the cadherin superfamily. The encoded protein contains six extracellular cadherin domains, a transmembrane domain, and a cytoplasmic tail differing from those of the classical cadherins. The encoded protein may play a role in the establishment and function of specific cell-cell connections in the brain. Human PCDH17 has the following nucleic acid sequence:

(SEQ ID NO: 6)
GATTTCGGGG GAGAGCCTTT TCCGAGGAAG AGAGGGAGGA
GCCTGGTGGG GAGAGGAAAC TACAAATCGG GACACTAGTT
CTTTACGCTG CATTTCCTCC CCTCCCTTTG GCTGCTCGGA
AAGGAGAGAG AGGAAAAAAA AAATACGCTT GGCTGGTAGA
TGCAGTCCGC CGCCGCCGCT GCCTCAGCCA GCAATGCAAG
ATTAGATCTC TAAATGCAGC AAAACACTGC CTGAAAACAG
ACCGGCCCGC GCAGCAAGCA GACATTTCAC GGTGCGCTGG
GGAAGCTTCA AAATATATCT GTGACTCTGT CTTCGTTGCT
CTTCATCCCC ATCAATTTCA TCACGGGAGG CGAGCAGCAA
GTAAGAATTT CACTTTCGGA TCTGCCTAGA GACACACCTC
CCTGCTCCCT CCCCCACTCG ATGTGAAGAG TATTCCGGAG
TCTCCGGGCG GGAGTAGATT TGCAGCACCC TAGCGGGAGC
GAGGAAAACC TACTGATTCT TTAGCTCATT ATCATCTCTC
CCAGACGAGA TTTCCTTCTT ATCGCCTGCC TCATCGCTCA
AGTTTGAGCC TCCCGAAGTC CGGGCGGGAG AGACGAAACC
CCTGGCTCAC CCCCAGCCGC AGGAAGCCAC CGCCTTGCTC
CAAGCCCCTG CAGCTCTGCT GCACCGCAGC TTCTCACCCA
GTGCGGATGC TGTAGATCAA CAGGTTCAGG GAACTTGAGC
AGAATAAGGA GAGACCACCG GGTGCCGCAG CTCGGGTGCA
GAGGGAAAAA AGGACCCATA GACTTGTGGC TCGCGTCGCG
CGCGCACGCT GCGCCAGGGC CCCAGGCTGG CGCGCACTCC
CTCTCTGGCT CCTCCAGTCC GATTGCTCCT GCCCCCACCT
TACAGGTCTG GGATGTACCT TTCCATCTGT TGCTGCTTTC
TTCTATGGGC CCCTGCCCTC ACTCTCAAGA ACCTCAACTA
CTCCGTGCCG GAGGAGCAAG GGGCCGGCAC GGTGATCGGG
AACATCGGCA GGGATGCTCG ACTGCAGCCT GGGCTTCCGC
CTGCAGAGCG CGGCGGCGGA GGGCGCAGCA AGTCGGGTAG
CTACCGGGTG CTGGAGAACT CCGCACCGCA CCTGCTGGAC
GTGGACGCAG ACAGCGGGCT CCTCTACACC AAGCAGCGCA
TCGACCGCGA GTCCCTGTGC CGCCACAATG CCAAGTGCCA
GCTGTCCCTC GAGGTGTTCG CCAACGACAA GGAGATCTGC
ATGATCAAGG TAGAGATCCA GGACATCAAC GACAACGCGC
CCTCCTTCTC CTCGGACCAG ATCGAAATGG ACATCTCGGA
GAACGCTGCT CCGGGCACCC GCTTCCCCCT CACCAGCGCA
CATGACCCCG ACGCCGGCGA GAATGGGCTC CGCACCTACC
TGCTCACGCG CGACGATCAC GGCCTCTTTG GACTGGACGT
TAAGTCCCGC GGCGACGGCA CCAAGTTCCC AGAACTGGTC
ATCCAGAAGG CTCTGGACCG CGAGCAACAG AATCACCATA
CGCTCGTGCT GACTGCCCTG GACGGTGGCG AGCCTCCACG
TTCCGCCACC GTACAGATCA ACGTGAAGGT GATTGACTCC
AACGACAACA GCCCGGTCTT CGAGGCGCCA TCCTACTTGG
TGGAACTGCC CGAGAACGCT CCGCTGGGTA CAGTGGTCAT
CGATCTGAAC GCCACCGACG CCGATGAAGG TCCCAATGGT
GAAGTGCTCT ACTCTTTCAG CAGCTACGTG CCTGACCGCG
TGCGGGAGCT CTTCTCCATC GACCCCAAGA CCGGCCTAAT
CCGTGTGAAG GGCAATCTGG ACTATGAGGA AAACGGGATG
CTGGAGATTG ACGTGCAGGC CCGAGACCTG GCGCCTAACC
CTATCCCAGC CCACTGCAAA GTCACGGTCA AGCTCATCGA
CCGCAACGAC AATGCGCCGT CCACCTGTTT CGTCTCCGTG
CGCCAGGGGG CGCTGAGCGA GGCCGCCCCT CCCGGCACCG
TCATCGCCCT GGTGCGGGTC ACTGACCGGG ACTCTGGCAA
GAACGGACAG CTGCAGTGTC GGGTCCTAGG CGGAGGAGGG
ACGGGCGGCG GCGGGGGCCT GGGCGGGCCC GGGGGTTCCG
TCCCCTTCAA GCTTGAGGAG AACTACGACA ACTTCTACAC
GGTGGTGACT GACCGCCCGC TGGACCGCGA GACACAAGAC
GAGTACAACG TGACCATCGT GGCGCGGGAC GGGGGCTCTC
CTCCCCTCAA CTCCACCAAG TCGTTCGCGA TCAAGATTCT
AGACGAGAAC GACAACCCGC CTCGGTTCAC CAAAGGGCTC
TACGTGCTTC AGGTGCACGA GAACAACATC CCGGGAGAGT
ACCTGGGCTC TGTGCTCGCC CAGGATCCCG ACCTGGGCCA
GAACGGCACC GTATCCTACT CTATCCTGCC CTCGCACATC
GGCGACGTGT CTATCTACAC CTATGTGTCT GTGAATCCCA
CGAACGGGGC CATCTACGCC CTGCGCTCCT TTAACTTCGA
GCAATGCAAG GCTTTTGAGT TCAAGGTGCT TGCTAAGGAC
TCGGGGGCGC CCGCGCACTT GGAGAGCAAC GCCACGGTGA
GGGTGACAGT GCTAGACGTG AATGACAACG CGCCAGTGAT
CGTGCTCCCC ACGCTGCAGA ACGACACCGC GGAGCTGCAG
GTGCCGCGCA ACGCTGGCCT GGGCTATCTG GTGAGCACTG
TGCGCGCCCT AGACAGCGAC TTCGGCGAGA GCGGGCGTCT
CACCTACGAG ATCGTGGACG GCAACGACGA CCACCTGTTT
GAGATCGACC CGTCCAGCGG CGAGATCCGC ACGCTGCACC
CTTTCTGGGA GGACGTGACG CCCGTGGTGG AGCTGGTGGT
GAAGGTGACC GACCACGGCA AGCCTACCCT GTCCGCAGTG
GCCAAGCTCA TCATCCGCTC GGTGAGCGGA TCCCTTCCCG
AGGGGGTACC ACGGGTGAAT GGCGAGCAGC ACCACTGGGA
CATGTCGCTG CCGCTCATCG TGACTCTGAG CACTATCTCC
ATCATCCTCC TAGCGGCCAT GATCACCATC GCCGTCAAGT
GCAAGCGCGA GAACAAGGAG ATCCGCACTT ACAACTGCCG
CATCGCCGAG TACAGCCACC CGCAGCTGGG TGGGGGCAAG
GGCAAGAAGA AGAAGATCAA CAAAAATGAT ATCATGCTGG
TGCAGAGCGA AGTGGAGGAG AGGAACGCCA TGAACGTCAT
GAACGTGGTG AGCAGCCCCT CCCTGGCCAC CTCCCCCATG
TACTTCGACT ACCAGACCCG CCTGCCCCTC AGCTCGCCCC
GGTCGGAGGT GATGTATCTC AAACCGGCCT CCAACAACCT
GACTGTCCCT CAGGGGCACG CGGGCTGCCA CACCAGCTTC
ACCGGACAAG GGACTAATGC AAGCGAGACC CCTGCCACTC
GGATGTCCAT AATTCAGACA GACAATTTTC CCGCAGAGCC
CAATTACATG GGCAGCAGGC AGCAGTTTGT TCAAAGTAGC
TCCACGTTTA AGGACCCAGA AAGAGCCAGC CTGAGAGACA
GTGGGCACGG GGACAGTGAT CAGGCTGACA GTGACCAAGA
CACTAACAAA GGCTCCTGCT GTGACATGTC TGTTAGGGAG
GCACTCAAGA TGAAAACTAC TTCAACTAAA AGCCAACCAC
TTGAACAAGA ACCAGAAGAG TGTGTTAATT GCACAGATGA
ATGCCGAGTG CTTGGTCATT CTGACAGGTG CTGGATGCCA
CAGTTCCCTG CAGCCAATCA GGCTGAAAAT GCAGATTACC
GCACAAATCT CTTTGTACCT ACAGTTGAAG CTAATGTTGA
GACTGAGACT TACGAAACTG TGAATCCCAC TGGGAAAAAG
ACTTTTTGTA CATTTGGAAA AGACAAGCGA GAGCACACTA
TTCTCATTGC CAACGTTAAA CCTTATTTAA AAGCCAAACG
TGCCCTGAGC CCTCTCCTCC AAGAGGTCCC CTCAGCATCA
AGCAGCCCAA CCAAGGCGTG CATCGAGCCT TGCACCTCAA
CAAAAGGCTC CCTGGATGGC TGTGAAGCAA AACCAGGAGC
CCTGGCTGAA GCAAGCAGTC AGTACTTGCC CACTGACAGT
CAATATCTGT CACCTAGTAA GCACTCAAGA GACCCTCCCT
TCATGGCTTC CGATCAGATG GCAAGGGTCT TTGCAGATGT
GCATTCCAGA GCCAGCCGGG ATTCCAGTGA GATGGGTGCT
GTTCTTGAGC AGCTTGACCA CCCCAACAGG GATCTGGGCA
GAGAGTCTGT GGATGCAGAG GAAGTTGTGA GAGAAATTGA
TAAGCTTTTG CAAGACTGCC GGGGAAACGA CCCTGTGGCT
GTGAGAAAGT GAAAAAAGAA AAAAAAAAAG GCATTGGCAT
TTTCTTGTCT CTTCTGTTGA TTTAAAAATG ATCCCTCCTG
GTGATAACCC ATTTTACAGG GATGAAGAAA GACCAATGCT
GCTTTAAGGC TTTTAGTGAA CATCTGAAGT GCCCACAAGT
ATGTTCTTTC CACTGCTGAT TTCTTTTTCA GAGATAACAA
TGGTTTCGTT TTGACCAAAC TTGTATTAGG ACAGAATTAA
TGATGCTTAA AGAGAAAAGA AAAAAAGAGA GAAGAAAAAG
GAGAGATGAA AAAGGAGGAT GAGGAGAAGA ATTACCTTTT
GACAATCTGT TAGGAAGGTA TGCAGTGTGA GAACTGAAGT
ATTTCTGATC ACTCTCAGAC TGTCCTCCGT GATTTATGCT
GACTTAACTG TTTACCTATA AACCCCATAC AAAGCAGGGT
CATAATTTGT GATCTGTGGT GGATTTCTAG CAGTCATCAC
AGGCTTCTAC TGAAAGTCCT GAAAAGACCT TGCAGTAGTC
CAAGCTACAC CAAACATTAA CACATATTTG TGGTAAACAT
TTCTGTATAA AGTTACCTGA CACACATATA AACACAAGGA
ACATTCCATA TCATTAGTCG AAAACAAAAA CAAAAAAAAA
ACCTTTGGTC ATTTGTAAGA CATCTCATGT CATATAAAAG
TTAAATGTAA AAAGATACAG TCCATTTTGT CCTGCACACA
CGTAGACTAA TTCACGTCAT TAAAGAAGAA GAAAACTTAA
AGATTTAAAA TGCCTATTTA GCATTTTAGT GTCCAACAAA
GATTTAAACA ATGATGAATA TGTTTTAAAT TTGACATAGA
AAAGTTCTAA AAAATAGTTA CCATTGAGTG GTAAGATTCA
GAGAAAATTA ACTTGATTAA TATGTTTTAT TCATTTGTGG
ACACTAAAAT AGCTCAGGAA AGTGAAAATG TCTTAGACAT
ACGCAAGTCA CATGACCATT TAAATGTGCA AATGTAAGAA
GATTCAATGT GTTTACATCA AATGACATAT TTTATTGATT
TATTGCAGAT TCAGTGCATA TGAGCCAAAT TGTTGAGTGT
GTAAGAGCTA TATTGTGTAT TTTATTAAAT TAATATATAG
TTGTGTTGCA AAAATATTTG GGCTTATATT GTAAATGGCA
AGTGTTGCCT TGGTAGCTGT CGAACTCTAT GAGTTTTGTT
TTTTCCTGCT TCCTTTTCCC CATGGAGTGT GGGAAGCAGT
GCCTCAGAGC AAAGTCTCTT GTTTAATGTA TAGTCTACCA
AGTACTACAG TACATAATCT GTTCAAAATG TGTTTGAGTG
AGCTGATGGA GCTAACTGAA AGGTCAAAAA TTACATCCAT
CAGTCATGGT TATGTGCAAG TCCTTGTAGA AGCTTTTATT
AAAGTCATGC TAAATCACAA GAATTGACAT TTGTACCAAT
ATCTGAAACT TCTTCATGTT TTTTCAATAA CATACAGCTT
CTGCCTGTGT AGATATTATG CCATCAGTTG GTTCTCAAAA
GTATTTTAAG TGCTTCAGAT GTGTGTTCCC ATTATATTTT
GAAAACATGA AAAATGCTTT AATGCATGTA TGTACCAGCA
GTGGTTACTT GCATTGTGTA GTGTTTTTCA AGAGGTCTGG
GTCTTAACAA AATGTTTTCC TTTATCTCAG TGCTCTTCTG
CCTCTTTTTG TTGGTGTCCT TTGAGAACAA TACACCTTCT
ATTCCTTCAT TTGGTTACAC CTTTCCTTGT GACATTTAGC
GAGTTTCAAA CTTACTTCCA TATGAGGCTA AGAAACCTCA
AATTTCAGGA ATTGGGAAAA ATAAAATTAG CACTTGCAGA
AGTAGCAGCA GATGGGAAAA TGCCTTGATT GACATTTTCT
TTCAGCATTT AAAATTTTTG GCATTTTACA GCTTCATGAC
AAACAGTTTT GTGCCCATAC CTTAGAAAAT GTGGTGCTGA
GTTAAATAAA GGCTGTTTGA GCACTGGAGC AGAAAAATGC
ATTATTTGCA AACTGGTGGA TAATTTTGTG CCTTCTCTTC
TGGCCACCAA GCCAGTGTAG AAACAGCAAA AATGTCATAA
AAATTCTTAT ATTTAAAACA AAAACAAAAG CAAAAACAAA
CATTGAATTA AATTAAGTTT TGTAATTTTA AACTTTAAAA
ACTTCTACTG AAAATATTTC CGCCAAATGC CATCAATATT
TTAGACTGTA CCTCGTTTGC AAAACTGCTT TGAGAGGGAA
GAGTGGACAA CTCCCATCAG CCTTATTCTC TTGAGAACTA
TATTTTGGTT CCTAGTAACA GCCTTTCCAA AGCTCTACTC
TTGGTTTTTA TTACTCATAA ATGTTTAAAT TAGAAAAGAA
GGGACCTTGT ACATGTGAAA CCTAATTGAC TCTCTATATT
TTGGACAATT TATGTATCTG AAATGTGTTG TCTCTGTTAT
ATGATGTTAT TTTTGCCAGG AGACTACAGG TTGATTTAGC
TTGATAGCTG AAATTTGATG GAAAACTGAT TTCCATTTAG
TCTTACCAAG TGTTGCTTCT CTCTTACTAG ACAGATATCC
ACTTAGTAAA ATCTAAAGCA GTATGTAAAT GAAACCAGCA
AAGAGAGTAG GGTTTATTTT ATAAACATTC TTAATGCTAA
GTAACCAGTT GTTCAATTTA TTATATGTGT CTGAGGACAT
TAAAACACCA TAAGGTTGTA ATAATTGGTT GTGCCAATGT
GTGAGGGATT TACCTTTAGG CTCTCTGTCA CCAGTGATTT
ACTAGTGTTA GCTGTTTAAC ACATTATCTG TATTTAGTAG
TGATTATTTA TTTACAAGTT GGTGGTAATT CAGCAGTCAG
GACTCTAAGC TTTTATAGTT GAATTGAGGA AATCTCGCTT
TTATTCATTT AGCTGGCAAC TGCCTTTATT GCAGACCTCT
GGTGCTTGGC TTTCAAGGAA GCCTATGAGA TGCCAAAATC
ACACCTTTAG AGAGCACCTT GCTCTAATAG GTGATGCATG
AGCAAACAGT GAGATTTGAA GGGGTTTTAA CATAATTTAG
AATGTGAAAA AAATATCAAT TCATATCTTT CAAGTACTAA
CCCCTCAAAA AAGCCCACAC ATACAAAATA TGTGATGTGA
TACCACTTTG TCTTTTAGGT CTTTAAGTAA CTGAAGTTAA
GCACAGAAAA AAAAATCACT TCATGGAAAT TTCAGTAAGA
AACCCAAACT TCTAAAAATT GCTTGCAGAT GAGCTAAAAA
AAAAAAAAAA AAAAAAAAGC AACAAAATAA CCTTTTCATC
AGAGTTAAAA GTAGTGAGAA TCTGTTAGTT ATACGTATAC
CAAAGTAAAC ATTAAAGAGA CATATCATGC AATTTCAAAG
AATTCTTTCA TGCTATTTCT TAACCTGACA TTTCTAACTT
TATTGCAGGC AATATACAAA GATTGGCTCA CTACTCCATA
GGTTAATTGA ATTCCTGGTT GAGAAACTAA CTTGTTTTGT
TTTCCAAAAT TAGCTGAAAT CTTGTAAAAC ATGACTTCCC
TTTAAAGGAT CTAGATATTG TTCAATTTAA AATATGGCAC
CATAAAAAAG TCATGTAGTA ATAGAGCATA TGCTTTTTTA
GAACCAGGTT AAAAGCTGTT TGTTATCTAA TAGAGTAAAA
GTTACTGAG

Human PCDH17 has the following amino acid sequence:

(SEQ ID NO: 32)
MYLSICCCFL LWAPALTLKN LNYSVPEEQG AGTVIGNIGR DARLQPGLPP AERGGGGRSK
SGSYRVLENS APHLLDVDAD SGLLYTKQRI DRESLCRHNA KCQLSLEVFA NDKEICMIKV
EIQDINDNAP SFSSDQIEMD ISENAAPGTR FPLTSAHDPD AGENGLRTYL LTRDDHGLFG
LDVKSRGDGT KFPELVIQKA LDREQQNHHT LVLTALDGGE PPRSATVQIN VKVIDSNDNS
PVFEAPSYLV ELPENAPLGT VVIDLNATDA DEGPNGEVLY SFSSYVPDRV RELFSIDPKT
GLIRVKGNLD YEENGMLEID VQARDLGPNP IPAHCKVTVK LIDRNDNAPS IGFVSVRQGA
LSEAAPPGTV IALVRVTDRD SGKNGQLQCR VLGGGGTGGG GGLGGPGGSV PFKLEENYDN
FYTVVTDRPL DRETQDEYNV TIVARDGGSP PLNSTKSFAI KILDENDNPP RFTKGLYVLQ
VHENNIPGEY LGSVLAQDPD LGQNGTVSYS ILPSHIGDVS IYTYVSVNPT NGAIYALRSF
NFEQTKAFEF KVLAKDSGAP AHLESNATVR VTVLDVNDNA PVIVLPTLQN DTAELQVPRN
AGLGYLVSTV RALDSDFGES GRLTYEIVDG NDDHLFEIDP SSGEIRTLHP FWEDVTPVVE
LVVKVTDHGK PTLSAVAKLI IRSVSGSLPE GVPRVNGEQH HWDMSLPLIV TLSTISIILL
AAMITIAVKC KRENKEIRTY NCRIAEYSHP QLGGGKGKKK KINKNDIMLV QSEVEERNAM
NVMNVVSSPS LATSPMYFDY QTRLPLSSPR SEVMYLKPAS NNLTVPQGHA GCHTSFTGQG
TNASETPATR MSIIQTDNFP AEPNYMGSRQ QFVQSSSTFK DPERASLRDS GHGDSDQADS
DQDTNKGSCC DMSVREALKM KTTSTKSQPL EQEPEECVNC TDECRVLGHS DRCWMPQFPA
ANQAENADYR TNLEVPTVEA NVETETYETV NPTGKKTFCT FGKDKREHTI LIANVKPYLK
AKRALSPLLQ EVPSASSSPT KACIEPCTST KGSLDGCEAK PGALAEASSQ YLPTDSQYLS
PSKQPRDPPF MASDQMARVF ADVHSRASRD SSEMGAVLEQ LDHPNRDLGR ESVDAEEVVR
EIDKLLQDCR GNDPVAVRK

Lymphocyte antigen 75 (LY75) acts as an endocytic receptor to direct captured antigens from the extracellular space to a specialized antigen-processing compartment. LY75 causes reduced proliferation of B-lymphocytes. Human LY75 has the following nucleic acid sequence:

(SEQ ID NO: 7)
GCGCTCAGCA GGCGGGGCGG GAGCCGCGTG CGCCCGAGGA CCCGGCCGGA AGGCTTGCGC
CAGCTCAGGA TGAGGACAGG CTGGGCGACC CCTCGCCGCC CGGCGGGGCT CCTCATGCTG
CTCTTCTGGT TCTTCGATCT CGCGGAGCCC TCTGGCCGCG CAGCTAATGA CCCCTTCACC
ATCGTCCATG GAAATACGGG CAAGTGCATC AAGCCAGTGT ATGGCTGGAT AGTAGCAGAC
GACTGTGATG AAACTGAGGA CAAGTTATGG AAGTGGGTGT CCCAGCATCG GCTCTTTCAT
TTGCACTCCC AAAAGTGCCT TGGCCTCGAT ATTACCAAAT CGGTAAATGA GCTGAGAATG
TTCAGCTGTG ACTCCAGTGC CATGCTGTGG TGGAAATGTG AGCACCACTC TCTGTACGGA
GCTGCCCGGT ACCGGCTGGC TCTGAAGGAT GGACATGGCA CAGCAATCTC AAATGCATCT
GATGTCTGGA AGAAAGGAGG CTCAGAGGAA AGCCTTTGTG ACCAGCCTTA TCATGAGATC
TATACCAGAG ATGGGAACTC TTATGGGAGA CCTTGTGAAT TTCCATTCTT AATTGATGGG
ACCTGGCATC ATGATTGCAT TCTTGATGAA GATCATAGTG GGCCATGGTG TGCCACCACC
TTAAATTATG AATATGACCG AAAGTGGGGC ATCTGCTTAA AGCCTGAAAA CGGTTGTGAA
GATAATTGGG AAAAGAACGA GCAGTTTGGA AGTTGCTACC AATTTAATAC TCAGACGGCT
CTTTCTTGGA AAGAAGCTTA TGTTTCATGT CAGAATCAAG GAGCTGATTT ACTGAGCATC
AACAGTGCTG CTGAATTAAC TTACCTTAAA GAAAAAGAAG GCATTGCTAA GATTTTCTGG
ATTGGTTTAA ATCAGCTATA CTCTGCTAGA GGCTGGGAAT GGTCAGACCA CAAACCATTA
AACTTTCTCA ACTGGGATCC AGACAGGCCC AGTGCACCTA CTATAGGTGG CTCCAGCTGT
GCAAGAATGG ATGCTGAGTC TGGTCTGTGG CAGAGCTTTT CCTGTGAAGC TCAACTGCCC
TATGTCTGCA GGAAACCATT AAATAATACA GTGGAGTTAA CAGATGTCTG GACATACTCA
GATACCCGCT GTGATGCAGG CTGGCTGCCA AATAATGGAT TTTGCTATCT GCTGGTAAAT
GAAAGTAATT CCTGGGATAA GGCACATGCG AAATGCAAAG CCTTCAGTAG TGACCTAATC
AGCATTCATT CTCTAGCAGA TGTGGAGGTG GTTGTCACAA AACTCCATAA TGAGGATATC
AAAGAAGAAG TGTGGATAGG CCTTAAGAAC ATAAACATAC CAACTTTATT TCAGTGGTCA
GATGGTACTG AAGTTACTCT AACATATTGG GAGAGGAATG AGCCAAATGT TCCCTACAAT
AAGACGCCCA ACTGTGCTGC CTACTTAGGA GAGCTAGGTC AGTGGAAAGT CCAATCATGT
GAGGAGAAAC TAAAATATGT ATGCAAGAGA AAGGGAGAAA AACTGAATGA CGCAAGTTCT
GATAAGATGT GTCCTCCAGA TGAGGGCTGG AAGAGACATG GAGAAACCTG TTACAAGATT
TATGAGGATG AGGTCCCTTT TGGAACAAAC TGCAATCTGA CTATCACTAG CAGATTTGAG
CAAGAATACC TAAAATATGT GATGAAAAAG TATGATAAAT CTCTAAGAAA ATACTTCTGG
ACTGGCCTGA GAGATGTAGA TTCTTGTGGA GAGTATAACT GGGCAACTGT TGGTGGAAGA
AGGCGGGCTG TAACCTTTTC CAACTGGAAT TTTCTTGAGC CAGCTTCCCC GGGCGGCTGC
GTGGCTATGT CTACTGGAAA GTCTGTTGGA AAGTGGGAGG TGAAGGACTG CAGAAGCTTC
AAAGCACTTT CAATTTGCAA GAAAATGAGT GGACCCCTTG GGCCTGAAGA AGCATCCCCT
AAGCCTGATG ACCCCTGTCC TGAAGGCTGG CAGAGTTTCC CCGCAAGTCT TTCTTGTTAT
AAGGTATTCC ATGCAGAAAG AATTGTAAGA AAGAGGAACT GGGAAGAAGC TGAACGATTC
TGCCAAGCCC TTGGAGCACA CCTTTCTAGC TTCAGCCATG TGGATGAAAT AAAGGAATTT
CTTCACTTTT TAACGGACCA GTTCAGTGGC CAGCATTGGC TGTGGATTGG TTTGAATAAA
AGGAGCCCAG ATTTACAAGG ATCCTGGCAA TGGAGTGATC GTACACCAGT GTCTACTATT
ATCATGCCAA ATGAGTTTCA GCAGGATTAT GACATCAGAG ACTGTGCTGC TGTCAAGGTA
TTTCATAGGC CATGGCGAAG AGGCTGGCAT TTCTATGATG ATAGAGAATT TATTTATTTG
AGGCCTTTTG CTTGTGATAC AAAACTTGAA TGGGTGTGCC AAATTCCAAA AGGCCGTACT
CCAAAAACAC CAGACTGGTA CAATCCAGAC CGTGCTGGAA TTCATGGACC TCCACTTATA
ATTGAAGGAA GTGAATATTG GTTTGTTGCT GATCTTCACC TAAACTATGA AGAAGCCGTC
CTGTACTGTG CCAGCAATCA CAGCTTTCTT GCAACTATAA CATCTTTTGT GGGACTAAAA
GCCATCAAAA ACAAAATAGC AAATATATCT GGTGATGGAC AGAAGTGGTG GATAAGAATT
AGCGAGTGGC CAATAGATGA TCATTTTACA TACTCACGAT ATCCATGGCA CCGCTTTCCT
GTGACATTTG GAGAGGAATG CTTGTACATG TCTGCCAAGA CTTGGCTTAT CGACTTAGGT
AAACCAACAG ACTGTAGTAC CAAGTTGCCC TTCATCTGTG AAAAATATAA TGTTTCTTCG
TTAGAGAAAT ACAGCCCAGA TTCTGCAGCT AAAGTGCAAT GTTCTGAGCA ATGGATTCCT
TTTCAGAATA AGTGTTTTCT AAAGATCAAA CCCGTGTCTC TCACATTTTC TCAAGCAAGC
GATACCTGTC ACTCCTATGG TGGCACCCTT CCTTCAGTGT TGAGCCAGAT TGAACAAGAC
TTTATTACAT CCTTGCTTCC GGATATGGAA GCTACTTTAT GGATTGGTTT GCGCTGGACT
GCCTATGAAA AGATAAACAA ATGGACAGAT AACAGAGAGC TGACGTACAG TAACTTTCAC
CCATTATTGG TTAGTGGGAG GCTGAGAATA CCAGAAAATT TTTTTGAGGA AGAGTCTCGC
TACCACTGTG CCCTAATACT CAACCTCCAA AAATCACCGT TTACTGGGAC GTGGAATTTT
ACATCCTGCA GTGAACGCCA CTTTGTGTCT CTCTGTCAGA AATATTCAGA AGTTAAAAGC
AGACAGACGT TGCAGAATGC TTCAGAAACT GTAAAGTATC TAAATAATCT GTACAAAATA
ATCCCAAAGA CTCTGACTTG GCACAGTGCT AAAAGGGAGT GTCTGAAAAG TAACATGCAG
CTGGTGAGCA TCACGGACCC TTACCAGCAG GCATTCCTCA GTGTGCAGGC GCTCCTTCAC
AACTCTTCCT TATGGATCGG ACTCTTCAGT CAAGATGATG AACTCAACTT TGGTTGGTCA
GAGGAGAAAC GTCTTCATTT TAGTCGCTGG GCTGAAACTA ATGGGCAACT CGAAGACTGT
GTAGTATTAG ACACTGATGG ATTCTGGAAA ACAGTTGATT GCAATGACAA TCAACCAGGT
GCTATTTGCT ACTATTCAGG AAATGAGACT GAAAAAGAGG TCAAACCAGT TGACAGTGTT
AAATGTCCAT CTCCTGTTCT AAATACTCCG TGGATACCAT TTCAGAACTG TTGCTACAAT
TTCATAATAA CAAAGAATAG GCATATGGCA ACAACACAGG ATGAAGTTCA TGCAGAATGC
CAGAAACTGA ATCCAAAATC ACATATTCTG AGTATTCGAG ATGAAAAGGA GAATAACTTT
GTTCTTGAGC AACTGCTGTA CTTCAATTAT ATGGCTTCAT GGGTCATGTT AGGAATAACT
TATAGAAATA AGTCTCTTAT GTGGTTTGAT AAGACCCCAC TGTCATATAC ACATTGGAGA
GCAGGAAGAC CAACTATAAA AAATGAGAAG TTTTTGGCTG GTTTAAGTAC TGACGGCTTC
TGGGATATTC AAACCTTTAA AGTTATTGAA GAAGCAGTTT ATTTTCACCA GCACAGCATT
CTTGCTTGTA AAATTGAAAT GGTTGACTAC AAAGAAGAAT ATAATACTAC ACTGCCACAG
TTTATGCCAT ATGAAGATGG TATCCACAGT GTTATTCAAA AAAAGGTAAC ATGGTATGAA
GCATTAAACA TGTGTTCTCA AAGTGGAGGT CACTTGGCAA GCGTTCACAA CCAAAATGGC
CAGCTCTTTC TGGAAGATAT TGTAAAACGT GATGGATTTC CACTATGGGT TGGGCTCTCA
AGTCATGATG GAAGTGAATC AAGTTTTGAA TGGTCTGATG GTAGTACATT TGACTATATC
CCATGGAAAG GCCAAACATC TCCTGGAAAT TGTGTTCTCT TGGATCCAAA AGGAACTTGG
AAACATGAAA AATGCAACTC TGTTAAGGAT GGTGCTATTT GTTATAAACC TACAAAATCT
AAAAAGCTGT CCCGTCTTAC ATATTCATCA AGATGTCCAG CAGCAAAAGA GAATGGGTCA
CGGTGGATCG AGTACAAGGG TCACTGTTAC AAGTCTGATC AGGCATTGCA CAGTTTTTCA
GAGGCCAAAA AATTGTGTTC AAAACATGAT CACTCTGCAA CTATCGTTTC CATAAAAGAT
GAAGATGAGA ATAAATTTGT GAGCAGACTG ATGAGGGAAA ATAATAACAT TACCATGAGA
GTTTGGCTTG GATTATCTCA ACATTCTGTT GACCAGTCTT GGAGTTGGTT AGATGGATCA
GAAGTGACAT TTGTCAAATG GGAAAATAAA AGTAAGAGTG GTGTTGGAAG ATGTAGCATG
TTGATAGCTT CAAATGAAAC TTGGAAAAAA GTTGAATGTG AACATGGTTT TGGAAGAGTT
GTCTGCAAAG TGCCTCTGGG CCCTGATTAC ACAGCAATAG CTATCATAGT TGCCACACTA
AGTATCTTAG TTCTCATGGG CGGACTGATT TGGTTCCTCT TCCAAAGGCA CCGTTTGCAC
CTGGCGGGTT TCTCATCAGT TCGATATGCA CAAGGAGTGA ATGAAGATGA GATTATGCTT
CCTTCTTTCC ATGACTAAAT TCTTCTAAAA GTTTTCTAAT TTGCACTAAT GTGTTATGAG
AAATTAGTCA CTTAAAATGT CCCAGTGTCA GTATTTACTC TGCTCCAAAG TAGAACTCTT
AAATACTTTT TCAGTTGTTT AGATCTTAGG CATGTGCTGG TATCCACAGT TAATTCCCTG
CTAAATGCCA TGTTTATCAC CCTAATTAAT AGAATGGAGG GGACTCCAAA GCTGGAACTG
AAGTCCAAAT TGTTTGTACA GTAATATGTT TAATGTTCAT TTTCTCTGTA TGAATGTGAT
TGGTAACTAG GATATGTATA TTTTAATAGA ATTTTTAACA AAACTTCTTA GAAAATTAAA
ATAGGCATAT TACTAGGTGA CATGTCTACT TTTTAATTTT TAAGAGCATC CGGCCAAATG
CAAAATTAGT ACCTCAAAGT AAAAATTGAA CTGTAAACTC TATCAGCATT GTTTCAAAAT
AGTCATTTTT AGCACTGGGG AAAAATAAAC AATAAGACAT GCTTACTTTT TAATTTTTAT
TTTTTTGAGA CTGAGTCTCT CTCTGTTGCC CAGGCTGGAG TACAATGGCG TGATCTCGGC
TCACTGCAAA TCTCCGCCTC CCAGGTTCAA GCGATTCTCC TGCCTCAGCC TCCTGAGTAG
CTGGGATTAC AGGCAACTGC CACCATGCCC GGCTAATTTT TGTATTTTTA GTAGAGATGG
GGTTTCACCA TGTTGGCCAG GCTGGTCTCG AACTCGTGAC CGCAGGTGAT CCTCCCGCCT
CGGCCTCCCA AAGTGCTGGG ATTACAGGCA TGAGCCACCG CGCCTGGCCT CTGCTTACTT
TTTATATAGC AAAATGATTC CACTTGGCAA GATGTTTCTT ATATTATTCC AAAGTTATTT
CATACCATTA TTATGTAAAT ATGAAGAGTT TTTTTCTGTT TATAATTGTT TATAAAACAA
TGACTTTTAA AGATTTAGTG CTTAACATTT TCCCAAGTGT GGGAACATTA TTTTTAGATT
GAGTAGGTAC CTTGTAGCAG TGTGCTTTGC ATTTTCTGAT GTATTACATG ACTGTTTCTT
TTGTAAAGAG AATCAACTAG GTATTTAAGA CTGATAATTT TACAATTTAT ATGCTTCACA
TAGCATGTCA ACTTTTGACT AAGAATTTTG TTTTACTTTT TTAACATGTG TTAAACAGAG
AAAGGGTCCA TGAAGGAAAG TGTATGAGTT GCATTTGTAA AAATGAGACT TTTTCAGTGG
AACTCTAAAC CTTGTGATGA CTACTAACAA ATGTAAAATT ATGAGTGATT AAGAAAACAT
TGCTTTGTGG TTATCACTTT AAGTTTTGAC ACCTAGATTA TAGTCTTAGT AATAGCATCC
ACTGGAAAAG GTGAAAATGT TTTATTCGGC ATTTAACTTA CATTTGTACT TTATTTTTGT
ATAAAATCCA TAGATTTATT TTACATTTAG AGTATTTACA CTATGATAAA GTTGTAAATA
ATTTTCTAAG ACAGTTTTTA TATAGTCTAC AGTTGTCCTG ATTTCTTATT GAATTTGTTA
GACTAGTTCT CTTGTCCTGT GATCTGTGTA CAATTTTAGT CACTAAGACT TTCCTCCAAG
AACTAAGCCA ACTTGATGTG AAAAGCACAG CTGTATATAA TGGTGATGTC ATAATAAAGT
TGTTTTATCT TTTAAGTAAA AGTAAAA

Human LY75 has the following amino acid sequence:

(SEQ ID NO: 33)
MRTGWATPRR PAGLLMLLFW FFDLAEPSGR AANDPFTIVH GNTGKCIKPV YGWIVADDCD
ETEDKLWKWV SQHRLFHLHS QKCLGLDITK SVNELRMFSC DSSAMLWWKC EHHSLYGAAR
YRLALKDGHG TAISNASDVW KKGGSEESLC DQPYHEIYTR DGNSYGRPCE FPFLIDGTWH
HDCILDEDHS GPWCATTLNY EYDRKWGICL KPENGCEDNW EKNEQFGSCY QFNTQTALSW
KEAYVSCQNQ GADLLSINSA AELTYLKEKE GIAKIFWIGL NQLYSARGWE WSDHKPLNFL
NWDPDRPSAP TIGGSSCARM DAESGLWQSF SCEAQLPYVC RKPLNNTVEL TDVWTYSDTR
CDAGWLPNNG FCYLLVNESN SWDKAHAKCK AFSSDLISIH SLADVEVVVT KLHNEDIKEE
VWIGLKNINI PTLFQWSDGT EVTLTYWDEN EPNVPYNKTP NCVSYLGELG QWKVQSCEEK
LKYVCKRKGE KLNDASSDKM CPPDEGWKRH GETCYKIYED EVPFGTNCNL TITSRFEQEY
LNDLMKKYDK SLRKYFWTGL RDVDSCGEYN WATVGGRRRA VTFSNWNFLE PASPGGCVAM
STGKSVGKWE VKDCRSFKAL SICKKMSGPL GPEEASPKPD DPCPEGWQSF PASLSCYKVF
HAERIVRKRN WEEAERFCQA LGAHLSSFSH VDEIKEFLHF LTDQFSGQHW LWIGLNKRSP
DLQGSWQWSD RTPVSTIIMP NEFQQDYDIR DCAAVKVFHR PWRRGWHFYD DREFIYLRPF
ACDTKLEWVC QIPKGRTPKT PDWYNPDRAG IHGPPLIIEG SEYWFVADLH LNYEEAVLYC
ASNHSFLATI TSFVGLKAIK NKIANISGDG QKWWIRISEW PIDDHETYSR YPWHRFPVTF
GEECLYMSAK TWLIDLGKPT DCSTKLPFIC EKYNVSSLEK YSPDSAAKVQ CSEQWIPFQN
KCFLKIKPVS LTFSQASDTC HSYGGTLPSV LSQIEQDFIT SLLPDMEATL WIGLRWTAYE
KINKWTDNRE LTYSNFHPLL VSGRLRIPEN FFEEESRYHC ALILNLQKSP FTGTWNFTSC
SERHFVSLCQ KYSEVKSRQT LQNASETVKY LNNLYKIIPK TLTWHSAKRE CLKSNMQLVS
ITDPYQQAFL SVQALLHNSS LWIGLFSQDD ELNFGWSDGK RLHFSRWAET NGQLEDCVVL
DTDGFWKTVD CNDNQPGAIC YYSGNETEKE VKPVDSVKCP SPVLNTPWIP FQNCCYNFII
TYNRHMATTQ DEVHTKCQKL NPKSHILSIR DEKENNFVLE QLLYFNYMAS WVMLGITYRN
KSLMWFDKTP LSYTHWRAGR PTIKNEKFLA GLSTOGFWDI QTFKVIEEAV YFHQHSILAC
KIEMVDYKEE YNTTLPQFMP YEDGIYSVIQ KKVTWYEALN MCSQSGGHLA SVHNQNGQLF
LEDIVKRDGF PLWVGLSSHD GSESSFEWSD GSTFDYIPWK GQTSPGNCVL LDPKGTWKHE
KCNSVKDGAI CYKPTKSKKL SRLTYSSRCP AAKENGSRWI QYKGHCYKSD QALHSFSEAK
KLCSKHDHSA TIVSIKDEDE NKFVSRLMRE NNNITMRVWL GLSQHSVDQS WSWLDGSEVT
FVKWENKSKS GVGRCSMLIA SNETWKKVEC EHGFGRVVCK VPLGPDYTAI AIIVATLSIL
VLMGGLIWFL FQRHRLHLAG FSSVRYAQGV NEDEIMLPSF HD

Placental growth factor (PGF) is a member of the VEGF (vascular endothelial growth factor) sub-family. PGF expression within human atherosclerotic lesions is associated with plaque inflammation and neovascular growth. The main source of PGF during pregnancy is the placental trophoblast. PGF is also expressed in many other tissues, including the villous trophoblast. Human PGF has the following nucleic acid sequence:

(SEQ ID NO: 8)
CTGCTGTCTG CGGAGGAAAC TGCATCGACG GACGGCCGCC CAGCTACGGG AGGACCTGGA
GTGGCACTGG GCGCCCGACG GACCATCCCC GGGACCCGCC TGCCCCTCGG CGCCCCGCCC
CGCCGGGCCG CTCCCCGTCG GGTTCCCCAG CCACAGCCTT ACCTACGGGC TCCTGACTCC
GCAAGGCTTC CAGAAGATGC TCGAACCACC GGCCGGGGCC TCGGGGCAGC AGTGAGGGAG
GCGTCCAGCC CCCCACTCAG CTCTTCTCCT CCTGTGCCAG GGGCTCCCCG GGGGATGAGC
ATGGTGGTTT TCCCTCGGAG CCCCCTGGCT CGGGACGTCT GAGAAGATGC CGGTCATGAG
GCTGTTCCCT TGCTTCCTGC AGCTCCTGGC CGGGCTGGCG CTGCCTGCTG TGCCCCCCCA
GCAGTGGGCC TTGTCTGCTG GGAACGGCTC GTCAGAGGTG GAAGTGGTAC CCTTCCAGGA
AGTGTGGGGC CGCAGCTACT GCCGGGCGCT GGAGAGGCTG GTGGACGTCG TGTCCGAGTA
CCCCAGCGAG GTGGAGCACA TGTTCAGCCC ATCCTGTGTC TCCCTGCTGC GCTGCACCGG
CTGCTGCGGC GATGAGAATC TGCACTGTGT GCCGGTGGAG ACGGCCAATG TCACCATGCA
GCTCCTAAAG ATCCGTTCTG GGGACCGGCC CTCCTACGTG GAGCTGACGT TCTCTCAGCA
CGTTCGCTGC GAATGCCGGC CTCTGCGGGA GAAGATGAAG CCGGAAAGGA GGAGACCCAA
GGGCAGGGGG AAGAGGAGGA GAGAGAAGCA GAGACCCACA GACTGCCACC TGTGCGGCGA
TGCTGTTCCC CGGAGGTAAC CCACCCCTTG GAGGAGAGAG ACCCCGCACC CGGCTCGTGT
ATTTATTACC GTCACACTCT TCAGTGACTC CTGCTGGTAC CTGCCCTCTA TTTATTAGCC
AACTGTTTCC CTGCTGAATG CCTCGCTCCC TTCAAGACGA GGGGCAGGGA AGGACAGGAC
CCTCAGGAAT TCAGTGCCTT CAACAACGTG AGAGAAAGAG AGAAGCCAGC CACAGACCCC
TGGGAGCTTC CGCTTTGAAA GAAGCAAGAC ACGTGGCCTC GTGAGGGGCA AGCTAGGCCC
CAGAGGCCCT GGAGGTCTCC AGGGGCCTGC AGAAGGAAAG AAGGGGGCCC TGCTACCTGT
TCTTGGGCCT CAGGCTCTGC ACAGACAAGC AGCCCTTGCT TTCGGAGCTC CTGTCCAAAG
TAGGGATGCG GATCCTGCTG GGGCCGCCAC GGCCTGGCTG GTGGGAAGGC CGGCAGCGGG
CGGAGGGGAT CCAGCCACTT CCCCCTCTTC TTCTGAAGAT CAGAACATTC AGCTCTGGAG
AACAGTGGTT GCCTGGGGGC TTTTGCCACT CCTTGTCCCC CGTGATCTCC CCTCACACTT
TGCCATTTGC TTGTACTGGG ACATTGTTCT TTCCGGCCAA GGTGCCACCA CCCTGCCCCC
CCTAAGAGAC ACATACAGAG TGGGCCCCGG GCTGGAGAAA GAGCTGCCTG GATGAGAAAC
AGCTCAGCCA GTGGGGATGA GGTCACCAGG GGAGGAGCCT GTGCGTCCCA GCTGAAGGCA
GTGGCAGGGG AGCAGGTTCC CCAAGGGCCC TGGCACCCCC ACAAGCTGTC CCTGCAGGGC
CATCTGACTG CCAAGCCAGA TTCTCTTGAA TAAAGTATTC TAGTGTGGAA AAAAAAAAAA
AAAAAAAAAA AAAAAAAA

Human PGF has the following amino acid sequence:

(SEQ ID NO: 34)
MPVMRLFPCF LQLLAGLALP AVPPQQWALS AGNGSSEVEV VFFQEVWGRS YCRALERLVD
VVSEYPSEVE HMFSPSCVSL LRCTGCCGDE NLHCVPVETA NVTMQLLKIR SGDRPSYVEL
TFSQHVRCEC RPLREKMKPE RRRPKGRGER RREKQRPTDC HLCGDAVPRR

Amyloid beta (A4) precursor protein-binding, family A, member 2 (APBA2) is a protein that in humans is encoded by the APBA2 gene. APBA2 is a member of the X11 protein family. It is a neuronal adaptor protein that interacts with the Alzheimer's disease amyloid precursor protein (APP). It stabilizes APP and inhibits production of proteolytic APP fragments including the A beta peptide that is deposited in the brains of Alzheimer's disease patients. APBA2 is believed to be involved in signal transduction processes. It is also regarded as a putative vesicular trafficking protein in the brain that can form a complex with the potential to couple synaptic vesicle exocytosis to neuronal cell adhesion. Human APBA2 has the following nucleic acid sequence:

(SEQ ID NO: 9)
CAGTCTCCTG AATATTTACG CGTTGCTGAA TCTCCTGTGG ACAAACCACC AATAGGCCAG
GACTGTCCTG TGGACAGACG GGGTGAGCCT CTTCTTGTGT CTGGAGATTC TGAAAAGATT
TGATCACCAG GAGATTTTTC GGGACATTAC CAAACCACTC ATTTTAGTGG CTGCCTCCGG
GTGATGATGG CTGTGTGAAC GACTGCCATG GCCCACCGGA AGCTTGAGAG CGTGGGGAGC
GGCATGTTGG ACCATAGGGT GAGACCAGGT CCTGTCCCTC ACAGCCAGGA GCCCGAGAGC
GAGGACATGG AGCTGCCCTT GCAGGGCTAT GTGCCCGAGG GCCTGGAGCT GGCTGCCCTG
CGGCCAGAGA GCCCCGCGCC AGAGGAACAG GAGTGCCACA ACCACAGCCC CGATGGGGAC
TCCAGCTCTG ACTACGTGAA CAACACCTCT GAGGAGGAGG ACTATGACGA GGGCCTCCCT
GAGGAGGAGG AGGGCATCAC CTACTACATC CGCTACTGCC CTGAGGACGA CAGCTACCTA
GAGGGCATGG ACTGCAACGG GGAGGAGTAC CTGGCCCACA GTGCACACCC TGTGGACACT
GATGAGTGCC AGGAGGCGGT GGAGGAGTGG ACGGACTCGG CGGGCCCGCA CCCCCACGGC
CACGAGGCTG AAGGCAGCCA GGACTACCCA GACGGCCAAC TGCCCATTCC GGAGGATGAG
CCCTCCGTCC TTGAGGCCCA TGACCAGGAA GAAGATGGTC ACTACTGTGC CAGCAAAGAG
GGCTACCAGG ACTACTACCC CGAGGAGGCC AACGGGAACA CCGGCGCCTC CCCCTACCGC
CTGAGGCGTG GGGATGGGGA CCTGGAGGAC CAGGAGGAGG ACATTGACCA GATCGTGGCA
GAGATCAAGA TGAGTCTGAG CATGACCAGC ATCACCAGCG CCAGTGAGGC CAGCCCCGAG
CATGGGCCTG AGCCAGGGCC TGAGGACTCT GTAGAGGCCT GCCCACCCAT CAAGGCCAGC
TGCAGCCCCA GCAGGCACGA GGCGAGGCCC AAGTCGCTGA ACCTCCTTCC CGAGGCCAAG
CACCCCGGAG ACCCCCAGAG AGGCTTCAAG CCCAAGACCA GGACCCCAGA AGAGAGGCTG
AAGTGGCCCC ACGAGCAGGT TTGCAATGGT CTGGAGCAGC CAAGGAAGCA GCAGCGCTCT
GATCTCAATG GACCTGTTGA CAATAACAAC ATTCCAGAGA CAAAGAAGGT GGCATCATTT
CCAAGTTTTG TGGCTGTTCC AGGGCCCTGC GAGCCAGAAG ACCTCATCGA CGGGATCATC
TTTGCTGCCA ATTACCTGGG GTCCACCCAG CTGCTATCAG AACGGAACCC TTCCAAAAAC
ATCAGAATGA TGCAAGCGCA GGAGGCCGTC AGCCGGGTCA AGAGGATGCA AAAGGCTGCT
AAGATCAAGA AAAAAGCGAA TTCTGAGGGG GATGCCCAGA CGCTGACGGA AGTGGACCTC
TTCATTTCCA CCCAGAGGAT CAAGGTTTTA AATGCAGACA CGCAGGAAAC CATGATGGAC
CACGCCTTGC GTACCATCTC CTACATCGCC GACATTGGGA ACATTGTAGT GCTGATGGCC
AGACGCCGCA TGCCCCGGTC AGCCTCTCAG GACTGCATCG AGACCACGCC CGGGGCCCAG
GAAGGCAAGA AGCAGTATAA GATGATCTGC CATGTGTTCG AGTCGGAGGA TGCCCAGCTC
ATCGCCCAGT CTATCGGCCA GGCCTTCAGC GTGGCCTACC AGGAGTTCCT GCGAGCCAAT
GGCATCAACC CCGAAGACTT GAGCCAGAAG GAATACAGCG ACATCATCAA CACCCAGGAG
ATGTACAACG ACGACCTCAT CCACTTCTCA AACTCGGAGA ACTGCAAGGA GCTGCAGCTG
GAGAAGCACA AGGGCGAGAT CCTGGGCGTG GTGGTGGTGG AGTCGGGCTG GGGCTCCATC
CTGCCCACGG TGATCCTGGC CAACATGATG AATGGCGGCC CGGCTGCCCG CTCGGGGAAG
CTGAGCATCG GGGACCAGAT CATGTCCATC AATGGCACCA GCCTGGTGGG GCTGCCCCTC
GCCACCTGCC AAGGCATCAT CAAGGGCCTG AAGAACCAGA CACAGGTGAA GCTCAACATT
GTCAGCTGTC CCCCGGTCAC CACGGTCCTT ATCAAGCGGC CAGACCTCAA GTACCAGCTG
GGCTTCAGCG TGCAGAATGG AATTATCTGC AGCCTCATGA GAGGGGGCAT TGCTGAGCGA
GGGGGCGTCC GTGTGGGCCA CCGCATCATC GAGATCAACG GGCAGAGCGT GGTGGCCACA
GCCCACGAGA AGATAGTCCA AGCTCTGTCC AACTCGGTCG GAGAGATCCA CATGAAGACC
ATGCCCGCCG CCATGTTCAG GCTCCTCACG GGTCAGGAGA CCCCGCTGTA CATCTAGGCC
ACCCCAGCCT GGCCACGCAG CCAGGACACC GGGCAGGGCC GCCCGGGCCC AGAGGAGCTG
GGAGCCGGGC CGCAGACTTG ACCCCGACGC CACAGCCCAG CCACGGACGC TGGCTCCCCA
AAGGGTGTGC CCTCACCACC CACTTGATTT TTTTCATTTT GCCAAAAAGG GGTATGTCTT
TATCAAAGGA GAGTCACAGA ACAAATGTTT GTTTGTAAAG CGTTCCAAGT ATTTTGCCAC
GTTCTGGACT GTCTTCTCCC TOCACAAGCC AGGGTGTGTC TCGGTAGCTG TGCGTGGTGT
GGAGTGTGTG TCTTTCCTCC CTGAAGCTGT GCGGAGCGAA CTGGCGCCTC CGAGGGACGC
GGCTCCCGGG GCAGGGCAGC CGTCACCCCT GCCTCCCGCC CCCTTGGCTG GGACGTCTGG
GGTCCTGTGG GGCCCCCACA ATGGTCCCAA ACAGCTGCCT CTGCCACTGA CTGCAGGGAC
ACGGGCAGCC TGGCTCCCAG GACACGACTT GTAATGAAAG TTTGGGGACA TGTGATTGAT
TGATTGATTG TAAATAAAGG ATGATGGCCA CAACATGAAA ACTCCATATT TATTTAGATG
CTATTATTAC TGTTTGGACT TTTATTTTGG CAGGCTTTTT TCCAGACTCT AGGGTTTTCC
AATGTGACTA ATGACCACAC CTGCCTCTCC CGTCGTCTCT TCTGGGCACC CTCCCACCCG
GCTGCATACC CGGGCAGGGC TCCCACAGAG ACAAGGAGGG CACAGGTGTC TGCCCCCTCT
TTAAAATCGA TCTACACACA TCCACGCACA TGCGACCCCG AGGAAACGAA ACCCACTCTA
GAAAACGCGA CCTTGGCCGC ACCTAAAGCA GCCAGCCGTG AGTGCAGACC CCTTGGCCAG
CGTGGCGCAG TGGCCCTGAG CAGTAGTGGC ATGTGTGTAG ATCAAGTCGG ATCTAGTCCA
GCTCGGTTCA TTAGCGATCC ATGTAATCTG ACGTCATCTT GTCTCGAAGT CTCTTTTTTT
GGCCCAGGCC TTGAAGAATA CACTGTGACT TAAGAAGCCT TACCACGCAG TAACTAAAGC
TTTAGGATGA CTGTATTCGA GGAGTGCCGT GTGTTGCATG CAGCTACCCG TAGGAAGACT
TCGCGCATAT CACTAATAAA CCTGAAGTCG TGATGAAAAA AAAAAAAAAA AAA

Human APBA2 has at least two isoforms. One isoform of human APBA2 has the following amino acid sequence:

(SEQ ID NO: 35)
MAHRKLESVG SGMLDHRVRP GPVPHSQEPE SEDMELPLEG YVPEGLELAA LRPESPAPEE
QECHNHSPDG DSSSDYVNNT SEEEDYDEGL PEEEEGITYY IRYCPEDDSY LEGMDCNGEE
YLAHSAHPVD TDECQEAVEE WTDSAGPHPH GHEAEGSQDY PDGQLPIPED EPSVLEAHDQ
EEDGHYCASK EGYQDYYPEE ANGNIGASPY RLRRGDGDLE DQEEDIDQIV AEIKMSLSMT
SITSASEASP EHGPEPGPED SVEACPPIKA SCSPSRHEAR PKSLNLLPEA KHPGDPQRGF
KPKTRTPEER LKWPHEQVCN GLEQPRKQQR SDLNGPVDNN NIPETKKVAS FPSEVAVPGP
CEPEDLIDGI IFAANYLGST QLLSERNPSK NIRMMQAQEA VSRVKRMQKA AKIKKKANSE
GDAQTLTEVD LFISTQRIKV LNADTQETMM DRALRTISYI ADIGNIVVLM ARRRMPRSAS
QDCIETTPGA QEGKKQYKMI CHVFESEDAQ LIAQSIGQAF SVAYQEFLRA NGINPEDLSQ
KEYSDIINTQ EMYNDDLIHF SNSENCKELQ LEKHKGEILG VVVVESGWGS ILPTVILANM
MNGGPAARSG KLSIGDQIMS INGTSLVGLP LATCQGIIKG LKNQTQVKLN IVSCPPVTTV
LIKRPDLKYQ LGFSVQNGII CSLMRGGIAE RGGVRVGHRI IEINGQSVVA TAHEKIVQAL
SNSVGEIHMK TMPAAMFRLL TGQETPLYI

Another isoform of human APBA2 has the following amino acid sequence:

(SEQ ID NO: 36)
MAHRKLESVG SGMLDHRVRP GPVPHSQEPE SEDMELPLEG YVPEGLELAA LRPESPAPEE
QECHNHSPDG DSSSDYVNNT SEEEDYDEGL PEEEEGITYY IRYCPEDDSY LEGMDCNGEE
YLAHSAHPVD TDECQEAVEE WTDSAGPHPH GHEAEGSQDY PDGQLPIPED EPSVLEAHDQ
EEDGHYCASK EGYQDYYPEE ANGNTGASPY RLRRGDGDLE DQEEDIDQIV AEIKMSLSMT
SITSASEASP EHGPEPGPED SVEACPPIKA SCSPSRHEAR PKSLNLLPEA KHPGDPQRGF
KPKTRTPEER LKWPHEQVCN GLEQPRKQQR SDLNGPVDNN NIPETKKVAS FPSFVAVPGP
CEPEDLIDGI IFAANYLGST QLLSERNPSK NIRMMQAQEA VSRVKNSEGD AQTLTEVDLF
ISTQRIKVLN ADTQETMMDH ALRTISYIAD IGNIVVLMAR RRMPRSASQD CIETTPGAQE
GKKQYKMICH VFESEDAQLI AQSIGQAFSV AYQEFLRANG INPEDLSQKE YSDIINTQEM
YNDDLIHFSN SENCKELQLE KHKGEILGVV VVESGWGSIL PTVILANMMN GGPAARSGKL
SIGDQIMSIN GTSLVGLPLA TCQGIIKGLK NQTQVKLNIV SCPPVTTVLI KRPDLKYQLG
FSVQNGIICS LMRGGIAERG GVRVGHRIIE INGQSVVATA HEKIVQALSN SVGEIHMKTM
PAAMFRLLTG QETPLYI

Prostaglandin E synthase (PTGES) is an enzyme that in humans is encoded by the PTGES gene. PTGES is a glutathione-dependent prostaglandin E synthase. The expression of this gene has been shown to be induced by proinflammatory cytokine interleukin 1 beta (IL1B). Its expression can also be induced by tumor suppressor protein TP53, and may be involved in TP53 induced apoptosis. Knockout studies in mice suggest that this gene may contribute to the pathogenesis of collagen-induced arthritis and mediate acute pain during inflammatory responses. Human PTGES has the following nucleic acid sequence:

(SEQ ID NO: 10)
GCTGCTCCTC TGTCGAGCTG ATCACACCCA CAGTTGAGCT GCGCTGGCCA GAGATGCCTG
CCCACAGCCT GGTGATGAGC AGCCCGGCCC TCCCGGCCTT CCTGCTCTGC AGCACGCTGC
TGGTCATCAA GATGTACGTG GTGGCCATCA TCACGGGCCA AGTGAGGCTG CGGAAGAAGG
CCTTTGCCAA CCCCGAGGAT GCCCTGAGAC ACGGAGGCCC CCAGTATTGC AGGAGCGACC
CCGACGTGGA ACGCTGCCTC AGGGCCCACC GGAACGACAT GGAGACCATC TACCCCTTCC
TTTTCCTGGG CTTCGTCTAC TCCTTTCTGG GTCCTAACCC TTTTGTCGCC TGGATGCACT
TCCTGGTCTT CCTCGTGGGC CGTGTGGCAC ACACCGTGGC CTACCTGGGG AAGCTGCGGG
CACCCATCCG CTCCGTGACC TACACCCTGG CCCAGCTCCC CTGCGCCTCC ATGGCTCTGC
AGATCCTCTG GGAAGCGGCC CGCCACCTGT GACCAGCAGC TGATGCCTCC TTGGCCACCA
GACCATGGGC CAAGAGCCGC CGTGGCTATA CCTGGGGACT TGATGTTCCT TCCAGATTGT
GGTGGGCCCT GAGTCCTGGT TTCCTGGCAG CCTGCTGCGC GTGTGGGTCT CTGGGCACAG
TGGGCCTGTG TGTGTGCCCG TGTGTGTGTA TGTGTGTGTG TATGTTTCTT AGCCCCTTGG
ATTCCTGCAC GAAGTGGCTG ATGGGAACCA TTTCAAGACA GATTGTGAAG ATTGATAGAA
AATCCTTCAG CTAAAGTAAC AGAGCATCAA AAACATCACT CCCTCTCCCT CCCTAACAGT
GAAAAGAGAG AAGGGAGACT CTATTTAAGA TTCCCAAACC TAATGATCAT CTGAATCCCG
GGCTAAGAAT GCAGACTTTT CAGACTGACC CCAGAAATTC TGGCCCAGCC AATCTAGAGG
CAAGCCTGGC CATCTGTATT TTTTTTTTTC CAAGACAGAG TCTTGCTCTG TTGCCCAAGC
TGGAGTGAAG TGGTACAATC TGGCTCACTG CAGCCTCCGC CTCCCGGGTT CAAGCGATTC
TCCCGCCTCA GCCTCCTGAG TAGCTGGGAT TACAGGCGCG TATCACCATA CCCAGCTAAT
TTTTGTATTT TTAGTAGAGA CGGGTTCACC ATGTTGCCCA GGAGGGTCTC GAACTCCTGG
CCTCAAGTGA TCCACCGGCC TCGGCCTCCC AAAGTGCTGG GATGACAGGC ATGAATCACT
GTGCTCAGCC ACCATCTGGA GTTTTAAAAG GCTCCCATGT GAGTCCCTGT GATGGCCAGG
CCAGGGGACC CCTGCCAGTT CTCTGTGGAA GCAAGGCTGG GGTCTTGGGT TCCTGTATGG
TGGAAGCTGG GTGAGCCAAG GACAGGGCTG GCTCCTCTGC CCCCGCTGAC GCTTCCCTTG
CCGTTGGCTT TGGATGTCTT TGCTGCAGTC TTCTCTCTGG CTCAGGTGTG GGTGGGAGGG
GCCCACAGGA AGCTCAGCCT TCTCCTCCCA AGGTTTGAGT CCCTCCAAAG GGCAGTGGGT
GGAGGACCGG GAGCTTTGGG TGACCAGCCA CTCAAAGGAA CTTTCTGGTC CCTTCAGTAT
CTTCAAGGTT TGGAAACTGC AAATGTCCCC TTGATGGGGA ATCCGTGTGT GTGTGTGTGT
GTGTGTGTGT GTGTGTGTGT GTGTGTGTGT GTTTTCTCCT AGACCCGTGA CCTGAGATGT
GTGATTTTTA GTCATTAAAT GGAAGTGTCT GCCAGCTGGG CCCAGCA

Human PTGES has the following amino acid sequence:

(SEQ ID NO: 37)
MPAHSLVMSS PALPAFLLCS TLLVIKMYVV AIITGQVRLR KKAFANPEDA LRHGGPQYCR
SDPDVERCLR AHRNDMETIY PFLFLGFVYS FLGPNPFVAW MHFLVFLVGR VAHTVAYLGK
LRAPIRSVTY TLAQLPCASM ALQILWEAAR EL

Myosin IF (MYO1F) is a protein that in humans is encoded by the MYO1F gene. Human MYO1F has the following nucleic acid sequence:

(SEQ ID NO: 11)
GTGAACGCGC AGAAGCAGGG CCATGCCCAA GCCACCCCCA AGATCCCCCT GAACCTGCAC
CTCCATCACG ACCCATTCAG GAGCCTCCAG GAGCCCAGAC ACCAGCCCCC CACCATGGGC
AGCAAGGAGC GCTTCCACTG GCAGAGCCAC AACGTGAAGC AGAGCGGCGT GGATGACATG
GTGCTTCTTC CCCAGATCAC CGAAGACGCC ATTGCCGCCA ACCTCCGGAA GCGCTTCATG
GACGACTACA TCTTCACCTA CATCGGCTCT GTGCTCATCT CTGTAAACCC CTTCAAGCAG
ATGCCCTACT TCACCGACCG TGAGATCGAC CTCTATCAGG GCGCGGCCCA GTATGAGAAT
CCCCCGCACA TCTACGCCCT CACGGACAAC ATGTACCGGA ACATGCTTAT CGACTGTGAG
AACCAGTGTG TCATCATTAG TGGAGAGAGT GGAGCTGGGA AGACAGTGGC AGCCAAATAT
ATCATGGGCT ACATCTCCAA GGTGTCTGGC GGAGGCGAGA AGGTCCAGCA CGTCAAAGAT
ATCATCCTGC AGTCCAACCC GCTGCTCGAG GCCTTCGGCA ACGCCAAGAC TGTGCGCAAC
AACAATTCCA GCCGCTTTGG CAAGTACTTT GAGATCCAGT TCAGCCGAGG TGGGGAGCCA
GATGGGGGCA AGATCTCCAA CTTCTTGCTG GAGAAGTCCC GCGTGGTCAT GCAAAATGAA
AATGAGAGGA ACTTCCACAT CTACTACCAG CTGCTGGAAG GGGCCTCCCA GGAGCAAAGG
CAGAACCTGG GCCTCATGAC ACCGGACTAC TATTACTACC TCAACCAATC GGACACCTAC
CAGGTGGACG GCACGGACGA CAGAAGCGAC TTTGGTGAGA CTCTGAGTGC TATGCAGGTT
ATTGGGATCC CGCCCAGCAT CCAGCAGCTG GTCCTGCAGC TCGTGGCGGG GATCTTGCAC
CTGGGGAACA TCAGTTTCTG TGAAGACGGG AATTACGCCC GAGTGGAGAG TGTGGACCTC
CTGGCCTTTC CCGCCTACCT GCTGGGCATT GACAGCGGGC GACTGCAGGA GAAGCTGACC
AGCCGCAAGA TGGACAGCCG CTGGGGCGGG CGCAGCGAGT CCATCAATGT GACCCTCAAC
GTGGAGCAGG CAGCCTACAC CCGTGATGCC CTGGCCAAGG GGCTCTATGC CCGCCTCTTC
GACTTCCTCG TCGAGGCCAT CAACCGTGCT ATGCAGAAAC CCCAGGAAGA GTACAGCATC
GGTGTGCTGG ACATTTACGG CTTCGAGATC TTCCAGAAAA ATGGCTTCGA GCAGTTTTGC
ATCAACTTCG TCAATGAGAA GCTGCAGCAA ATCTTTATCG AACTTACCCT GAAGGCCGAG
CAGGAGGAGT ATGTGCAGGA AGGCATCCGC TGGACTCCAA TCCAGTACTT CAACAACAAG
GTCGTCTGTG ACCTCATCGA AAACAAGCTG AGCCCCCCAG GCATCATGAG CGTCTTGGAC
GACGTGTGCG CCACCATGCA CGCCACGGGC GGGGGAGCAG ACCAGACACT GCTGCAGAAG
CTGCAGGCGG CTGTGGGGAC CCACGAGCAT TTCAACAGCT GGAGCGCCGG CTTCGTCATC
CACCACTACG CTGGCAAGGT CTCCTACGAC GTCAGCGGCT TCTGCGAGAG GAACCGAGAC
GTTCTCTTCT CCGACCTCAT AGAGCTGATG CAGACCAGTG AGCAGGCCTT CCTCCGGATG
CTCTTCCCCG AGAAGCTGGA TGGAGACAAG AAGGGGCGCC CCAGCACCGC CGGCTCCAAG
ATCAAGAAAC AACCCAACGA CCTGGTGGCC ACACTGATGA GGTGCACACC CCACTACATC
CGCTGCATCA AACCCAACGA GACCAAGAGG CCCCGAGACT GGGAGGAGAA CAGAGTCAAG
CACCAGGTGG AATACCTGGG CCTGAAGGAG AACATCAGGG TGCGCAGAGC CGGCTTCGCC
TACCGCCGCC AGTTCGCCAA ATTCCTGCAG AGGTATGCCA TTCTGACCCC CGAGACGTGG
CCGCGGTGGC GTGGGGACGA ACGCCAGGGC GTCCAGCACC TGCTTCGGGC GGTCAACATG
GAGCCCGACC AGTACCAGAT GGGGAGCACC AAGGTCTTTG TCAAGAACCC AGAGTCGCTT
TTCCTCCTGG AGGAGGTGCG AGAGCGAAAG TTCGATGGCT TTGCCCGAAC CATCCAGAAG
GCCTGGCGGC GCCACGTGGC TGTCCGGAAG TACGAGGAGA TGCGGGAGGA AGCTTCCAAC
ATCCTGCTGA ACAAGAAGGA GCGGAGGCGC AACAGCATCA ATCGGAACTT CGTCGGGGAC
TACCTGGGGC TGGAGGAGCG GCCCGAGCTG CGTCAGTTCC TGGGCAAGAG GGAGCGGGTG
GACTTCGCCG ATTCGGTCAC CAAGTACGAC CGCCGCTTCA AGCCCATCAA GCGGGACTTG
ATCCTGACGC CCAAGTGTGT GTATGTGATT GGGCGAGAGA AAGTGAAGAA GGGACCTGAG
AAGGGCCAGG TGTGTGAAGT CTTGAAGAAG AAAGTGGACA TCCAGGCTCT GCGGGGAGTC
TCCCTCAGCA CGCGACAGGA CGACTTCTTC ATCCTCCAAG AGGATGCCGC CGACAGCTTC
CTGGAGAGCG TCTTCAAGAC CGAGTTTGTC AGCCTTCTGT GCAAGCGCTT CGAGGAGGCG
ACGCGGAGGC CCCTGCCCCT CACCTTCAGC GACACACTAC AGTTTCGGGT GAAGAAGGAG
GGCTGGGGCG GTGGCGGCAC CCGCAGCGTC ACCTTCTCCC GCGGCTTCGG CGACTTGGCA
GTGCTCAAGG TTGGCGGTCG GACCCTCACG GTCAGCGTGG GCGATGGGCT GCCCAAGAGC
TCCAAGCCTA CGCGGAAGGG AATGGCCAAG GGAAAACCTC GGAGGTCGTC CCAAGCCCCT
ACCCGGGCGG CCCCTGCGCC CCCCAGAGGC ATGGATCGCA ATGGGGTGCC CCCCTCTGCC
AGAGGGGGCC CCCTGCCCCT GGAGATCATG TCTGGAGGGG GCACCCACAG GCCTCCCCGG
GGCCCTCCGT CCACATCCCT GGGAGCCAGC AGACGACCCC GGGCACGTCC GCCCTCAGAG
CACAACACAG AATTCCTCAA CGTGCCTGAC CAGGGCATGG CCGGCATGCA GAGGAAGCGC
AGCGTGGGGC AACGGCCAGT GCCTGGTGTG GGCCGACCCA AGCCCCAGCC TCGGACACAT
GGTCCCAGGT GCCGGGCCCT ATACCAGTAC GTGGGCCAAG ATGTGGACGA GCTGAGCTTC
AACGTGAACG AGGTCATTGA GATCCTCATG GAAGATCCCT CGGGCTGGTG GAAGGGCCGG
CTTCACGGCC AGGAGGGCCT TTTCCCAGGA AACTACGTGG AGAAGATCTG AGCTGGGCCC
TGGGATACTG CCTTCTCTTT CGCCCGCCTA TCTGCCTGCC GGCCTGGTGG GGAGCCAGGC
CCTGCCAATG AGAGCCTCGT TTACCTGGGC TGCAATAGCC TAAAAGTCCA GTCCTTTGGC
CTCCAGTCCT GCCCAGGCCC TGGGTCACCA GGTCACTGCT GCAGCCCCCG CCCCTGGGCC
CTGGTCTTCC TCCAACATCA CACCTGCTGC CCATTCTCCA TTTCTGTGTG TGTCAAAGGG
GACTAACAGC AGAATCTACC TCCCAACTGC CATGTGATTA AGAAATGGGT CTTGAGTCCT
GTGCTGTTGG CAAAGTGCCA GGCACAGTTG GGGAGGGGGG GGTCCTTAAC AAGCGTGACT
TTGCTCATTC TGTCATCACT AAGGCAATAA ACCTTTGCCA GGTGAAAAAA AAAAAAAAAA
AAAAAAAAAA AAAAAAAAAA

Human MYO1F has the following amino acid sequence:

(SEQ ID NO: 38)
MGSKERFHWQ SHNVKQSGVD DMVLLPQITE DAIAANLRKR FMDDYIFTYI GSVLISVNPF
KQMPYFTDRE IDLYQGAAQY ENPPHIYALT DNMYRNMLID CENQCVIISG ESGAGKTVAA
KYIMGYISKV SGGGEKVQHV KDIILQSNPL LEAFGNAKTV RNNNSSRFGK YFEIQFSRGG
EPDGGKISNE LLEKSRVVMQ NENERNFHIY YQLLEGASQE QRQNLGLMTP DYYYYLNQSD
TYQVDGTDDR SDFGETLSAM QVIGIPPSIQ QLVLQLVAGI LHLGNISFCE DGNYARVESV
DLLAFPAYLL GIDSGRLQEK LTSRKMDSRW GGRSESINVT LNVEQAAYTR DALAKGLYAR
LFDFLVEAIN RAMQKPQEEY SIGVLDIYGF EIFQKNGFEQ FCINFVNEKL QQIFIELTLK
AEQEEYVQEG IRWTPIQYFN NKVVCDLIEN KLSPPGIMSV LDDVCATMHA TGGGADQTLL
QKLQAAVGTH EHENSWSAGF VIHHYAGKVS YDVSGFCERN RDVLFSDLIE LMQTSEQAFL
RMLFPEKLDG DKKGRPSTAG SKIKKQANDL VATLMRCTPH YIRCIKPNET KRPRDWEENR
VKHQVEYLGL KENIRVRRAG FAYRROFAKF LQRYAILTPE TWPRWRGDER QGVQHLLRAV
NMEPDQYQMG STKVFVKNPE SLFLLEEVRE RKFDGFARTI QKAWRRHVAV RKYEEMREEA
SNILLNKKER RRNSINRNFV GDYLGLEERP ELRQFLGKRE RVDFADSVTK YDRRFKPIKR
DLILTPKCVY VIGREKVKKG PEKGQVCEVL KKKVDIQALR GVSLSTRQDD FFILQEDAAD
SFLESVFKTE FVSLLCKRFE EATRRPLPLT FSDTLQFRVK KEGWGGGGTR SVTFSRGFGD
LAVLKVGGRT LTVSVGDGLP KSSKEIRKGM AKGKPRRSSQ APTRAAPAPP RGMDRNGVPP
SARGGPLPLE IMSGGGTHRP PRGPPSTSLG ASRRPRARPP SEHNTEFLNV PDQGMAGMQR
KRSVGQRPVP GVGRPKPQPR THGPRCRALY QYVGQDVDEL SFNVNEVIEI LMEDPSGWWK
GRLHGQEGLF PGNYVEKI

G protein-coupled receptor 84 (GPR84) is a protein that in humans is encoded by the GPR84 gene. Human GPR84 has the following nucleic acid sequence:

(SEQ ID NO: 12)
TAACTGTCCA CCAGAAAGGA CTGCTCTTTG GGTGAGTTGA ACTTCTTCCA TTATAGAAAG
AATTGAAGGC TGAGAAACTC AGCCTCTATC ATGTGGAACA GCTCTGACGC CAACTTCTCC
TGCTACCATG AGTCTGTGCT GGGCTATCGT TATGTTGCAG TTAGCTGGGG GGTGGTGGTG
GCTGTGACAG GCACCGTGGG CAATGTGCTC ACCCTACTGG CCTTGGCCAT CCAGCCCAAG
CTCCGTACCC GATTCAACCT GCTCATAGCC AACCTCACAC TGGCTGATCT CCTCTACTGC
ACGCTCCTTC AGCCCTTCTC TGTGGACACC TACCTCCACC TGCACTGGCG CACCGCTGCC
ACCTTCTGCA GGGTATTTGG GCTCCTCCTT TTTGCCTCCA ATTCTGTCTC CATCCTGACC
CTCTGCCTCA TCGCACTGGG ACGCTACCTC CTCATTGCCC ACCCTAAGCT TTTTCCCCAA
GTTTTCAGTG CCAAGGGGAT AGTGCTGGCA CTGGTGAGCA CCTGGGTTGT GGGCGTGGCC
AGCTTTGCTC CCCTCTGGCC TATTTATATC CTGGTACCTG TAGTCTGCAC CTGCAGCTTT
GACCGCATCC GAGGCCGGCC TTACACCACC ATCCTCATGG GCATCTACTT TGTGCTTGGG
CTCAGCAGTG TTGGCATCTT CTATTGCCTC ATCCACCGCC AGGTCAAACG AGCAGCACAG
GCACTGGACC AATACAAGTT GCGACAGGCA AGCATCCACT CCAACCATGT GGCCAGGACT
GATGAGGCCA TGCCTGGTCG TTTCCAGGAG CTGGACAGCA GGTTAGCATC AGGAGGACCC
AGTGAGGGGA TTTCATCTGA GCCAGTCAGT GCTGCCACCA CCCAGACCCT GGAAGGGGAC
TCATCAGAAG TGGGAGACCA GATCAACAGC AAGAGAGCTA AGCAGATGGC AGAGAAAAGC
CCTCCAGAAG CATCTGCCAA AGCCCAGCCA ATTAAAGGAG CCAGAAGAGC TCCGGATTCT
TCATCGGAAT TTGGGAAGGT GACTCGAATG TGTTTTGCTG TGTTCCTCTG CTTTGCCCTG
AGCTACATCC CCTTCTTGCT GCTCAACATT CTGGATGCCA GAGTCCAGGC TCCCCGGGTG
GTCCACATGC TTGCTGCCAA CCTCACCTGG CTCAATGGTT GCATCAACCC TGTGCTCTAT
GCAGCCATGA ACCGCCAATT CCGCCAAGCA TATGGCTCCA TTTTAAAAAG AGGGCCCCGG
AGTTTCCATA GGCTCCATTA GAACTGTGAC CCTAGTCACC AGAATTCAGG ACTGTCTCCT
CCAGGACCAA AGTGGCCAGG TAATACGAGA ATAGGTGAAA TAACACATGT GGGCATTTTC
ACAACAATCT CTCCCCAGCC TCCCAAATCA AGTCTCTCCA TCACTTGATC AATGTTTCAG
CCCTAGACTG CCCAAGGAGT ATTATTAATT ATTAATAAAT GAATTCTGTG CTTTTAAAAA
AAAAAAAATA AAAAAAGAAA AAAAAAAAAA AAAAAAAAAA AAAAAA

Human GPR84 has the following amino acid sequence:

(SEQ ID NO: 39)
MWNSSDANFS CYHESVLGYR YVAVSWGVVV AVTGTVGNVL TLLALAIQPK LRTRFNLLIA
NLTLADLLYC TLLQPFSVDT YLHLHWRTGA TFCRVFGLLL FASNSVSILT LCLIALGRYL
LIAEPKLFPQ VFSAKGIVLA LVSTWVVGVA SFAPLWPIYI LVPVVCTCSF DRIRGRPYTT
ILMGIYFVLG LSSVGIFYCL IHRQVKRAAQ ALDQYKLRQA SIHSNHVART DEAMPGRFQE
LDSRLASGGP SEGISSEPVS AATTQTLEGD SSEVGDQINS KRAKQMAEKS PPEASAKAQP
IKGARRAPDS SSEFGKVTRM CFAVFLCFAL SYIPFLLLNI LDARVQAPRV VHMLAANLTW
LNGCINPVLY AAMNRQFRQA YGSILKRGPR SFHRLH

Transcription elongation factor A (SII)-like 2 (TCEAL2) is a protein that in humans is encoded by the TCEAL2 gene. TCEAL2 is a member of the transcription elongation factor A (SII)-like (TCEAL) gene family. Members of this family contain TFA domains and may function as nuclear phosphoproteins that modulate transcription in a promoter context-dependent manner. Multiple family members are located on the X chromosome. Human TCEAL2 has the following nucleic acid sequence:

(SEQ ID NO: 15)
AGCGGTCGGG TCCGGGCGCC CGCGCAGAAT CAGCTGTCTG AGCTGCCCAG GCGGCGGGGG
AGCAGCGAGC GGGCTTCAGC GAGCCGCAGG AGGCACAGGC CTGTCCTGGG TCCCCGCAGG
TCTGCGCGTC TGTTGTTCCC AGCGCTCTGA GAGGCCTGAA AAGGAAGAGC AACCTGTCCA
GAATCCCCGC AGGAAAGGAA AAGGAGGGGA AATCTCGACA TGGAAAAACT CTTCAATGAA
AATGAAGGAA TGCCTTCGAA TCAAGGAAAG ATAGACAATG AAGAACAGCC ACCGCACGAG
GGAAAGCCAG AAGTAGCTTG TATTCTGGAA GACAAGAAGT TAGAAAACGA GGGAAACACA
GAAAACACGG GCAAGAGAGT TGAGGAACCG TTAAAGGATA AAGAAAAGCC AGAGAGTGCG
GGAAAGGCAA AAGGAGAAGG AAAGTCAGAG AGGAAGGGAA AGTCAGAGAT GCAGGGAGGA
TCAAAGACAG AGGGAAAGCC AGAGAGAGGG GGAAGGGCAG AGGGTGAAGG AGAGCCAGAC
AGTGAAAGAG AGCCAGAGAG TGAGGGAGAG CCAGAAAGTG AAACAAGGGC TGCAGGAAAG
CGCCCAGCTG AGGATGATAT ACCCAGGAAA GCCAAAAGAA AAACCAACAA GGGGCTGGCT
CAGTACCTCA AGCAATATAA GGAAGCCATA CATGATATGA ATTTCAGCAA TGAGGACATG
ATAAGAGAAT TTGACAACAT GGCTAGGGTG GAGGATAAAA GGAGAAAAAG CAAACAGAAA
TTGGGGGCGT TTTTGTGGAT GCAAAGAAAT TTACAGGACC CCTTCTATCC TAGGGGTCCA
AGGGAATTCA GGGGTGGCTG CAGGGCCCCA CGAAGGGACA CTGAAGACAT TCCTTATGTG
TAGTGTCCCT GGCAGGCATT TGTCAGGCCA TATGTTTTAA CCTTATGGTA ATACTTTGCT
TTAGTCGTTC CTCCTGCTAC CAGTAGCGTT TTGACCCACC TGCCAGTGTT TGCTTGCTCT
ATGTTTCAGT AGCAGATTTT CACACATGTG CATTGCAGAG ACGTCATGAT TCGTGGAAAA
ATAAAGCAGC TTATAATATC AAAAAAAAAA AAAAAAAAAA AAA

Human TCEAL2 has the following amino acid sequence:

(SEQ ID NO: 40)
MEKLFNENEG MPSNQGKIDN EEQPPHEGKP EVACILEDKK LENEGNTENT GKRVEEPLKD
KEKPESAGKA KGEGKSERKG KSEMQGGSKT EGKPERGGRA EGEGEPDSER EPESEGEPES
ETRAAGKRPA EDDIPRKAKR KTNKGLAQYL KQYKEAIHDM NFSNEDMIRE FDNMARVEDK
RRKSKQKLGA FLWMQRNLQD PFYPRGPREF RGGCRAPRRD TEDIPYV

Collagen, type XXIII, alpha 1 (COL23A1) is a protein that in humans is encoded by the COL23A1 gene. Collagen XXIII is predicted to be a type II membrane protein consisting of an amino-terminal cytoplasmic domain, a transmembrane region, and three collagenous domains flanked by short noncollagenous domains. Collagen XXIII is a new member of the transmembrane collagen family, showing structural homology with the transmembrane collagens XIII and XXV. Human COL23A1 has the following amino acid sequence:

(SEQ ID NO: 16)
AGAGGTGCGC GCTGCGCGTG GGATCAGCCC GGCGCCGACG GGTGGCTCCG AGGAGCTCGC
TCCTTCCTCG CCCCCGCCCC CTCGCCGCGC GGGGCCAGCC CGGCCGCTCC TCCCCTGGGT
GGGTCCCTGC TCCTTTTCTG GCAGGGTCTA TTTGCATAGA GGAAACTGCC CAAAGTGGCC
GCTGTGGAGG AGCTGGCTGC GGCGAAGGGG GCGTGCGCGG CGATCCGCTG CTACCCGGAG
GCTAACCCCC GCGCCCGGCG GACCTCGTGC CTCGGGCTGT CCCGCCTGCT CCTCTCGCAC
CCAGCCTCTG CCCCAGCAGC ACCGCCCCCT CGGAGAGTCC ACGCGCGACG AACGCGCCAT
GGGCCCAGGC GAGCGCGCCG GTGGCGGCGG CGACGCGGGG AAGGGCAATG CGGCGGGCGG
CGGCGGCGGA GGGCGCTCGG CGACGACGGC CGGGTCCCGG GCGGTGAGCG CGCTGTGCCT
GCTGCTCTCC GTGGGCTCGG CGGCTGCCTG CCTGCTGCTG GGTGTCCAGG CGGCCGCGCT
GCAGGGCCGG GTGGCGGCGC TCGAGGAGGA GCGGGAGCTG CTGCGGCGCG CGGGGCCGCC
AGGCGCCCTG GACGCCTGGG CCGAGCCGCA CCTGGAGCGC CTGCTGCGGG AGAAGTTGGA
CGGACTAGCG AAGATCCGGA CTGCTCGGGA AGCTCCATCC GAATGTGTCT GCCCCCCAGG
GCCCCCTGGA CGGCGCGGCA AGCCTGGGAG AAGAGGCGAC CCTGGTCCTC CAGGGCAATC
AGGACGAGAT GGCTACCCGG GACCCCTGGG TTTGGATGGC AAGCCCGGAC TTCCAGGCCC
GAAAGGGGAA AAGGGTGCAC CAGGAGACTT TGGCCCCCGG GGAGACCAAG GACAAGATGG
AGCTGCTGGG CCTCCGGGGC CCCCTGGACC TCCTGGGGCC CGGGGCCCTC CTGGCGACAC
TGGGAAAGAT GGCCCCAGGG GAGCACAAGG CCCAGCGGGC CCCAAAGGAG AGCCCGGACA
AGACGGCGAG ATGGGCCCAA AGGGACCCCC AGGGCCCAAG GGTGAGCCTG GAGTACCTGG
AAAGAAGGGC GACGATGGGA CACCAAGCCA GCCTGGACCA CCAGGGCCCA AGGGCGAGCC
AGGGAGCATG GGGCCTCGGG GAGAGAACGG TGTGGACGGT GCCCCAGGAC CGAAGGGGGA
GCCTGGCCAC CGAGGCACGG ATGGAGCTGC AGGGCCCCGG GGTGCCCCAG GCCTCAAGGG
CGAGCAGGGA GACACAGTGG TGATCGACTA TGATGGCAGG ATCTTGGATG CCCTCAAGGG
GCCTCCCGGA CCACAGGGGC CCCCAGGGCC ACCAGGGATC CCTGGAGCCA AGGGCGAGCT
TGGATTGCCC GGTGCCCCAG GAATCGATGG AGAGAAGGGC CCCAAAGGAC AGAAAGGAGA
CCCAGGAGAG CCTGGGCCAG CAGGACTCAA AGGGGAAGCA GGCGAGATGG GCTTGTCCGG
CCTCCCGGGC GCTGACGGCC TCAAGGGGGA GAAGGGGGAG TCGGCGTCTG ACAGCCTACA
GGAGAGCCTG GCTCAGCTCA TAGTGGAGCC AGGGCCCCCT GGCCCCCCTG GCCCCCCAGG
CCCGATGGGC CTCCAGGGAA TCCAGGGTCC CAAGGGCTTG GATGGAGCAA AGGGAGAGAA
GGGTGCGTCG GGTGAGAGAG GCCCCGACGG CCTGCCTGGG CCAGTTGGCC CACCGGGCCT
TATTGGGCTG CCAGGAACCA AAGGAGAGAA GGGCAGACCC GGGGAGCCAG GACTAGATGG
TTTCCCTGGA CCCCGAGGAG AGAAAGGTGA TCGGAGCGAG CGTGGAGAGA AGGGAGAACG
AGGGGTCCCC GGCCGGAAAG GAGTGAAGGG CCCGATGGGC GAGCCGGGAC CACCGGGCCT
GGACCAGCCG TGTCCCGTGG GCCCCGACGG GCTGCCTGTG CCTGGCTGCT GGCATAAGTG
ACCCACAGGC CCAGCTCACA CCTGTACAGA TCCGTGTGGA CATTTTTAAT TTTTGTAAAA
ACAAAACAGT AATATATTGA TCTTTTTTCA TGGAATGCGC TACCTGTGGC CTTTTAACAT
TCAAGAGTAT GCCCACCCAG CCCCAAAGCC ACCGGCATGT GAAGCTGCCG GAAAGTGGAC
AGGCCAGACC AGGGAGATGT GTACCTGAGG GGCACCCTTG GGCCTGGGCT TTCCCAGGAA
GGAGATGAAG GTAGAAGCAC CTGGCTCGGG CAAGGCTAGA AAGATGCTAC GTTGGGCCTT
CAGTCACCTG ATCAGCAGAG AGACTCTCAG CTGTGGTACT GCCCTGTAAG AACCTGCCCC
CGCAAAACTC TGGAGTCCCT GGGACACACC CTATCCAAGA AGACCCAGGG GTGGAACAGC
GGCTGCTGTT GCTCCTGGCC TCATCAGCCT CCAAACTCAA CCACAACCAG CTGCCTCTGC
AGTTGGACAA GACTTGGCCC CCGGACAAGA CTCGCCCAGC ACTTGCGGCT GGGCCCGGGG
AGCAGTGAGT GGAAATCCCC CACGAGGGTC TAGCTCTACC ACATTCAGGA GGCCTCAGGA
GGCCAGCCTG CCATGAGAGC ACATGTCCTC TGGCCAGGAG TAGTGGCTGA GCTCTGTGAT
CGCTGTGATG TGGACCCAGC TCCAGGGAGC AGAGTGTCGA GGATGGAGGG GGCCAGCCTG
GACTGACTGC TACTTCCTGT CTCTGTTTCC ATTATCACCC AGAGAGGGAC AAGATAGGAC
ATGGCCTGGA CCAGGGAGGC AGGCCTCCCA CTCAGAGTCT GGGTCTCACT GGCCCCAAGT
CTCCCACCCA GAACTCTGGC CAAAAATGGC TCTCTAGGTG GGCTGTGCAG GCAAAGCAAA
GCTCAGGGCT GGTTCCCAGC TGGCCTGAGC AGGGGGCCTG CCACCAGACC CACCCACGCT
CTGACGAGAG GCTTTTCCAC CTCCAGCAAG TGTTCCCAGC AACCAGCTCC ATCCTGGCTG
CTTGCCTTCC ATTTCCGTGT AGATGGAGAT CACTGTGTGT AATAAACCAC AAGTGCGTGT
CTGAAAAAAA AAAAAAAAAA

Human COL23A1 has the following amino acid sequence:

(SEQ ID NO: 41)
MGPGERAGGG GDAGKGNAAG GGGGGRSATT AGSRAVSALC LLLSVGSAAA CLLLGVQAAA
LQGRVAALEE ERELLRRAGP PGALDAWAEP HLERLLREKL DGLAKIRTAR EAPSECVCPP
GPPGRRGKPG RRGDPGPPGQ SGRDGYPGPL GLDGKPGLPG PKGEKGAPGD FGPRGDQGQD
GAAGPPGPPG PPGARGPPGD TGKDGPRGAQ GPAGPKGEPG QDGEMGPKGP PGPKGEPGVP
GKKGDDGTPS QPGPPGPKGE PGSMGPRGEN GVDGAPGPKG EPGHRGTDGA AGPRGAPGLK
GEQGDTVVID YDGRILDALK GPPGPQGPPG PPGIPGAKGE LGLPGAPGID GEKGPKGQKG
DPGEPGPAGL KGEAGEMGLS GLPGADGLKG EKGESASDSL QESLAQLIVE PGPPGPPGPP
GPMGLQGIQG PKGLDGAKGE KGASGERGPS GLPGPVGPPG LIGLPGTKGE KGRPGEPGLD
GFPGPRGEKG DRSERGEKGE RGVPGRKGVK GQKGEPGPPG LDQPCPVGPD GLPVPGCWHK

ST8 alpha-N-acetyl-neuraminide alpha-2,8-sialyltransferase 4 (ST8SIA4) (RefSeq ID: NM_{—}005668) is an enzyme that in humans is encoded by the ST8SIA4 gene. The protein encoded by this gene catalyzes the polycondensation of alpha-2,8-linked sialic acid required for the synthesis of polysialic acid, a modulator of the adhesive properties of neural cell adhesion molecule (NCAM1). The encoded protein, which is a member of glycosyltransferase family 29, is a type II membrane protein that may be present in the Golgi apparatus. Two transcript variants encoding different isoforms have been found for this gene.

Human ST8SIA4 has the following amino acid sequence:

(SEQ ID NO: 17)
TGACGCCCCC GAACCCAGCT GCAGAAGCTG CCGCCACCTC CAATGCACAA GGTGTCTCAT
CTGAAAAGAA ACCTGAGCCC CAGGGAGGCG GCGCGGAGCG ACCCTGGCAG AGCTGGCGCA
AACAGGGCGA GAGGTCGCTG GGCAGCGTTC GAGGACCAGA GGGAGCTCGG CCACAGAAGA
CCCCAGTGAT CTGATCCCGG GATCCCGGCT CCAAGCTCTC CTCGCATTTT ACAGATTTCA
CCCCCGCGAC TATCTCCCCA AAACGGAGCC TTTATATCAA GAGAAGGTGC GGGAGCTGGG
GCAACCAGGA CTTTCTCGGG CACCCAAGAT GCGCTCCATT AGGAAGAGGT GGACGATCTG
CACAATAAGT CTGCTCCTGA TCTTTTATAA GACAAAAGAA ATAGCAAGAA CTGAGGAGCA
CCAGGAGACG CAACTCATCG GAGATGGTGA ATTGTCTTTG AGTCGGTCAC TTGTCAATAG
CTCTGATAAA ATCATTCGAA AGGCTGGCTC TTCAATCTTC CAGCACAATG TAGAAGGTTG
GAAAATCAAT TCCTCTTTGG TCCTAGAGAT AAGGAAGAAC ATACTTCGTT TCTTAGATGC
AGAACGAGAT GTGTCAGTGG TCAAGAGCAG TTTTAAGCCT GGTGATGTCA TACACTATGT
GCTTGACAGG CGCCGGACAC TAAACATTTC TCATGATCTA CATAGCCTCC TACCTGAAGT
TTCACCAATG AAGAATCGCA GGTTTAAGAC CTGTGCAGTT GTTGGAAATT CTGGCATTCT
GTTAGACAGT GAATGTGGAA AGGAGATTGA CAGTCACAT TTTGTAATAA GGTGTAATCT
AGCTCCTGTG GTGGAGTTTG CTGCAGATGT GGGAACTAAA TCAGATTTTA TTACCATGAA
TCCATCAGTT GTACAAAGAG CATTTGGAGG CTTTCGAAAT GAGAGTGACA GAGAAAAATT
TGTGCATAGA CTTTCCATGC TGAATGACAG TGTCCTTTGG ATTCCTGCTT TCATGGTCAA
AGGAGGAGAG AAGCACGTGG AGTGGGTTAA TGCATTAATC CTTAAGAATA AACTGAAAGT
GCGAACTGCC TATCCGTCAT TGAGACTTAT TCATGCTGTC AGAGGTTACT GGCTGACCAA
CAAAGTTCCT ATCAAAAGAC CCAGCACAGG TCTTCTCATG TATACACTTG CCACAAGATT
CTGTGATGAA ATTCACCTGT ATGGATTCTG GCCCTTCCCT AAGGATTTAA ATGGAAAAGC
GGTCAAATAT CATTATTATG ATGACTTAAA ATATAGGTAC TTTTCCAATG CAAGCCCTCA
CAGAATGCCA TAAGAATACA AAACATTAAA TGTGCTACAT AATAGAGGAG CTCTAAAACT
GACAACAGGA AAGTGTGTAA AGCAATAAAG CACATTTTGA AACAAACAAT ATGCACTTCT
TTTCTGAAGA TGCTTCCGAA GATTTGAAAA TAGGATCCAA AACACGGCTG GGTTTCAGCA
TCCACCAATG AACTGAAAGG TGAATAAAGG ACGTTCATGA GAAATCGACT ACCAGCTGAT
GAAATACCTG CAAAGTGCTC TAAAAATTAA ATATTTTGAC TTTAAGGGTC CTAGTAAGTG
CCACTTCCAC TAAGAATACA GTTTGAATGT ATAATCAGTA GTGTTTACAA GATCCAACAG
TGCACTCATC ATTAGTTAAC AAAGCAAATA TGTTCATCAC TGTCAGGCTG CCCACAGCAA
CACCAAGCAT ATTAGAAGAG GAACCCCAGG AACGCAACTC AGACCTTGGG AAATTAAACC
ATCCTTGTCA GCAGAAGCCA AGATGGAAGC AGTTTGAGCA ATGAAATCCG TAAGATTAAA
CAACTCAAGT AAATGCTTCA GTCAGGACTC TGAGTCTGAT CATGAATTTT ATGTTTTAAT
TTATGTTTTT TTTTTGTCTT CTGGAATCTC TTTTGGTTTG GATATTGGGA TGCTTAGAAA
TCCTTTCTGA GATGCATATG AGTGAGGAAA TAAACTTTAA GTAATTATTT TTAAAGTTCT
TATACTTTTT AAAAGCTATC ACACAAAGAC TTTTTTTTTT TTTTTTGTCT CGCTCTGTTG
CCCAGGCTGG AGTACAGTGG CGCGATCTCA GCTCACTGCA AGCTCCGCCT CCCAAGTTCA
CTCCATTCTC CTGCCTCAGC CTCCGGAGTA GCTGGGACTG CAGGCGCCTG CCACCACGCC
TGGCTAATTT TTTGTATTTT TAGTGGAGAC GGATTTTCAC CGTGTTAGCC AGGATGGTCT
CAATCTCCTG ACCTCGTGAT CCACCCGCCT TGGCCTCCCA AAGTGCTGGG ATTACAGGCG
TGAGCCACCG TGCCTGGCCG ACATTTTTAA AAAAGTTTTA TTTTGCACGG CTCTAAACCT
CCATGTTATT TTCCAGTGGT GTAGAAGGTA CCAGCTAAAG TGAACCACTA TGTAATATTA
GGCCATTCTA AAGGAAAGAT GTTCCATGTC ATCAGAGATG GTAAAATAGG CCGGGAAAAA
AAAATCTTTG GTACCAAAGA TTACACTTGT GTTTCTACAC AGCAAACCAT TTTTCTTTCA
TGAAAATAAT ATATTATTAA CATGAATATA TTATTTTGCT ATTAATGTGA AAGTTGTCTC
TAAATATTTT TTAATTTTCA AACTCATACT TTATTTTCAT TTGAAATGTT TTTCACACCT
TTTGCATTAC ATAATAATTT TGTGGAAGCA TTTTGCCCTT TAGAATAAAT ATTAGATTGA
TATAGCTGAA ATGTGACTTC CAGTTCTTTG ATATTCCCCT TGTTATTCAA ATAGAAATAT
GGAAATGCTT TATATATTAC TGTTAAATTT CTTAGTGCAG AAATAACATT ATTAATAGAG
TATTGTTTTC AAAACAGAGA TGATTAATTT CAAGAGGTTT AACAGTGAAA TTGTGTCAAT
ATTTTGCATT TAAAATGAAT TTAATTGACC GATATTTTCT GTAGTTAAAT TTAGTCACAA
TATCACATAT GTTCTTCAAG AAACACATGA AATTATTAAT AAAGTAATTA AAAAATTTTT
AATGTATAAC AGAATTGACC AATAGGCCAG TTTTCTGGTA ACTTATGATA GTAGATTGTT
TCTTTAGAAA CTGGGCAGAA GCTCTGCATT CTCACTTGTA CTTTGATTTC TTATTTCTTG
GGCAGGCAAT TTGAGGAAAG AAGAAATGGC ATGGGGAATA TATATGTTTT GTTTCTTAGG
GAAAACAGTC TGAGAAATGA ATAAAAAGCA TGAAGTACGT GTGTGTGTGT GTGTGTTACC
ATGGAAAAGG ATATTCACAG TAGTACAGTT CTCAATATTT TTAATTAGAT GTCATATTTT
TTTAATATAG TAAAACCTTG GGATATACAA TATTACATCT TTTGAGAATG TATGTGTCTC
TAAGTAAGTA AAATCTAATG CGTATAGGAG ACTGATAGCT AAAAATGAAT GGAACATTAA
TGTACTTTTA TAATTAAACC TCTTATCTAT CAGAAATTGT AAGAGAATAG ATACATGTTT
TGAATGTAAA GTTGAAAAGT CTGGTTTACT TAATAAATTG AAAGTGATTT ATAAAAAGCA
AATTTGGACT ACTTGCAAAT GATAAGCTAT TCTAGTAGCC TTTAGTTTAA ATCCAACAGA
AATCTAGAAG TCACAAGCAA ATATCTTAAA GGTAAAATCC ATCTGGGCAC TCAGTTAAAG
TATATCTTAA AAAAGCAGCA GCAAGGTACC TTGCCATTTT TAGCATATTT TCTTCCTTTT
TCTTTTTTCT TTTTTTTTTT TTTTTGAGAT GGAGTCTCAC TCTGTCACAC AGGCTGGAAT
GCAGTGATGC CATCTCAGCT CACTGCAACC TCCACCTCCT GGGTTCAAGT GATTCTCGTG
CCTCAGCCTC CCAAGTAGCT GGGGTTACAG GCGCCCACCA CCACACTCGG CTAATTTTGT
GTTTTTAGTA GAGACAAAGT TTCACCATGT TGGCCAGGCT GGTCTTGAAC TTCCTGACCT
CAGGTTATCC ACCCACCTCA GCCTCCCAAA GTGCTGGGAT TACAGGTGTG AGCCACCGCA
GCCGGACCAT TTTTAGTATA TTTTCAGTAA ATACATTTAA ACAATGTTAA GGCCACAGCA
CACATATCTC AGCCATTCAT TGTTCTGTGC ATTGATGTTT ATCTCATAGA TGCATTGAGT
AGTGCCTTTT TAGCTTTTTC ACATTACTTT GTCACCATAT CCTTTGTGTT CTCTAAATAC
ATTGCCCACT TCCAAAAATG TTCAGCATGA AAAAAAGGGC TTCAGTGTCG ATTGAGATTG
CTTTTGTTCA TCTCAGGGAT TTCAATAGTC AAGAATGAAT TCAGTTAAAG GTATTTAGGG
TTCAAAGAAG ACAAACTGTA CAAGCCCATT TCATTCCTTG TTGTATACCT TTCCATCTGC
CCTCCCATTT TAACTATCTA CTGTGGCCTT TTTATGGAAA CAGAGCAAGA TCAATGAAGG
CTAATGGCAA GAATAAGAAA AAGAGTTGAG ATTTAACCAA TAGCGGAGCA TAAAGGATCA
TGACAAAATC AAATTATAAA AGCATACTTG AAATAGGTGG AGCTTTTTCT TTTGAAAATA
TATATTCACA ATTTTAATAT TTTAATTTAT TTTTTACTAT TTAACCCTGT ACTTGGCAAT
GCTCAGGCAG CTGATTGTGA AATATTCTTG TCCTTTACAG AACATGGTTG TTATTGTGCT
GTTGACATGA ATAGACCATG GAAACATTTT CATCATTATT ATTCAGCCTG TGCTGTAGTT
AATGTTAAGT TGCTGAAATA AAAAGTGAGC AAGTAATAGA TTTTCTTGGC AAATCTAATG
ATTCAGCCCA CAGGACTGTT GAAACTACTG CGGAAGTTTT TCTATCTGAA AGAAGGTGCT
GGGCATTCAA ATGTGTTCAT GTATTGTATA TCATATGAAT TGTATATCAA TTACTAATGG
GAATTTCTAC ATATATGCTT ACAAAAGCAA TTTATTTAAG TAATGCTAGG GGTAGTGTAC
ATACCAATTA GTTATTCAGC TCCTTTACAG AAAAAGGATG AACAAATTAA TTTATTTCTA
ATTGAGCCAG TTAGACATAA TGCATATAAC GTGATATTTG GTTCATGAAA GAGTTGTTTT
CATGTGGTTA TTGTAGGGAG TATATATAAT TGTGGAAGGG GTATGGGAAG AGTTGTGTAT
AGTTAGTTGT TATCTCTACA AGTTTGAAAG TTTTCCCATC AAACATTATC AATATACCAA
TGTTTTAAAA ATTGAGTGAG GGTTATTATT TGTATTTGAT GAAAGAAAAT CCAAATAAAG
CCCACCTAGA AATAGATATT TTATTATATA TGTGCTATAG ATATACCTAT ATAGTACAAA
TAGACATGTG TGATGCATAT ATACAATGTT ATATATGTGT ATATGTCTGT ATACACACTG
AGTCTGTAAT ATGTATACAC TAAATTTGTG TTATGCTAAC ATCTTCAGGG TCTGCACTGT
GAACTCCCCT GGAGATAAGT AAGTCCACTT TAGAATAAAG AAGTTCTTTT GAGACTTCAG
TTACTAACGT GCTTTAAGAG GTATCTACTT TATAACTGAA TTCTATGTCG TTCATACGTA
GAGTTACAGT AAGGGTCTAG TATGTCCAAA TCTTAATAAT AAAGAAGAAA AGTAAAGGCT
TCAAGCTAGC AATGTATTCG AATTACAGTT TTCAGATTGT GGCTCCAGGC CTTGTGTTTC
TCATTTAAGT AGCACCTTTT AATAAAAACC GTTTCTTTGT GTAGGCAAAA GCACAAGTGT
TTCAAATGTA AATAGCAGGA AAAAAAAAGA GTTTACAGAG ATAGCATTGC TGCACAGAAT
AATTGCTACT GAGTATTTCT TATAGAATTT GTGGAACTGA AAGATGAGGT TTATTCTGTC
AAGTTCAAGT TCATTCTGTT CAACACTGTT TTCTTATTGT TTGTGTATAG CAACCGGGTA
TTATTGTTTT ATCATTTGTA AAATTGTAAA ATAAATTAAT CCCTTTTTTT CACTGTTTCT
CTTATCTCAT ATATCCAAGC CCTTGGTTAT ACTTTGTATG TCAATGTTAG GTGATCATTT
TTAACAAGCT TTGGCTTGTG CTTTGCTTTT CCACTCCCCT TAGCCCTAGT GGTTGGCAAT
TAGGCAAACC ATTTATTTTT AAGTGTATAC ATGGGAATAT GAACAATGTC AAAAACCCCA
TGAATATTAG GAAATCCTTA ACGATATTTT GTGTAGCACA TTCTGTTTGC GGTTGAGGGA
ATAAAGTATT TCACAAGTGA AAAAAAAAAA

Human ST8SIA4 has at least two isoforms. One isoform of human ST8SIA4 has the following amino acid sequence:

(SEQ ID NO: 42)
MRSIRKRWTI CTISLLLIFY KTKEIARTEE HQETQLIGDG ELSLSRSLVN SSDKIIRKAG
SSIFQHNVEG WKINSSLVLE IRKNILRFLD AERDVSVVKS SFKPGDVIHY VLDRRRTLNI
SHDLHSLLPE VSPMKNRRFK TCAVVGNSGI LLDSECGKEI DSHNFVIRCN LAPVVEFAAD
VGTKSDFITM NPSVVQRAFG GFRNESDREK FVHRLSMLND SVLWIPAFMV KGGEKHVEWV
NALILKNKLK VRTAYPSLRL IHAVRGYWLT NKVPIKRPST GLLMYTLATR FCDEIHLYGF
WPFPKDLNGK AVKYHYYDDL KYRYFSNASP HRMPLEFKTL NVLHNRGALK LTTGKCVKQ

Another isoform of human ST8SIA4 has the following amino acid sequence:

(SEQ ID NO: 43)
MRSIRKRWTI CTISLLLIFY KTKEIARTEE HQETQLIGDG ELSLSRSLVN SSDKIIRKAG
SSIFQHNVEG WKINSSLVLE IRKNILRFLD AERDVSVVKS SFKPGDVIHY VLDRRRTLNI
SHDLHSLLPE VSPMKNRRFK TCAVVGNSGI LLDSECGKEI DSHNFVIR

Matrix metallopeptidase 8 (MMP8) is a protein encoded by the MMP8 gene. MMP8 is a collagen cleaving enzyme which is present in the connective tissue of most mammals. Proteins of the matrix metalloproteinase (MMP) family are involved in the breakdown of extracellular matrix in normal physiological processes, such as embryonic development, reproduction, and tissue remodeling, as well as in disease processes, such as arthritis and metastasis. Most MMP's are secreted as inactive proproteins which are activated when cleaved by extracellular proteinases. However, the enzyme encoded by this gene is stored in secondary granules within neutrophils and is activated by autolytic cleavage. Its function is degradation of type I, II and III collagens. Human MMP8 has the following amino acid sequence:

(SEQ ID NO: 19)
GACACATGAT GCTGTGAACG TCAGGGTGCT CGCCAGGGAA GGGCCCTACC CAGAGGGACA
GAAAGAAAGC CAGGAGGGGT AGAGTTTGAA GAGAAGATCA TGTTCTCCCT GAAGACGCTT
CCATTTCTGC TCTTACTCCA TGTGCAGATT TCCAAGGCCT TTCCTGTATC TTCTAAAGAG
AAAAATACAA AAACTGTTCA GGACTACCTG GAAAAGTTCT ACCAATTACC AAGCAACCAG
TATCAGTCTA CAAGGAAGAA TGGCACTAAT GTGATCGTTG AAAAGCTTAA AGAAATGCAG
CGATTTTTTG GGTTGAATGT GACGGGGAAG CCAAATGAGG AAACTCTGGA CATGATGAAA
AAGCCTCGCT GTGGAGTGCC TGACAGTGGT GGTTTTATGT TAACCCCAGG AAACCCCAAG
TGGGAACGCA CTAACTTGAC CTACAGGATT CGAAACTATA CCCCACAGCT GTCAGAGGCT
GAGGTAGAAA GAGCTATCAA GGATGCCTTT GAACTCTGGA GTGTTGCATC ACCTCTCATC
TTCACCAGGA TCTCACAGGG AGAGGCAGAT ATCAACATTG CTTTTTACCA AAGAGATCAC
GGTGACAATT CTCCATTTGA TGGACCCAAT GGAATCCTTG CTCATGCCTT TCAGCCAGGC
CAAGGTATTG GAGGAGATGC TCATTTTGAT GCCGAAGAAA CATGGACCAA CACCTCCGCA
AATTACAACT TGTTTCTTGT TGCTGCTCAT GAATTTGGCC ATTCTTTGGG GCTCGCTCAC
TCCTCTGACC CTGGTGCCTT GATGTATCCC AACTATGCTT TCAGGGAAAC CAGCAACTAC
TCACTCCCTC AAGATGACAT CGATGGCATT CAGGCCATCT ATGGACTTTC AAGCAACCCT
ATCCAACCTA CTGGACCAAG CACACCCAAA CCCTGTGACC CCAGTTTGAC ATTTGATGCT
ATCACCACAC TCCGTGGAGA AATACTTTTC TTTAAAGACA GGTACTTCTG GAGAAGGCAT
CCTCAGCTAC AAAGAGTCGA AATGAATTTT ATTTCTCTAT TCTGGCCATC CCTTCCAACT
GGTATACAGG CTGCTTATGA AGATTTTGAC AGAGACCTCA TTTTCCTATT TAAAGGCAAC
CAATACTGGG CTCTGAGTGG CTATGATATT CTACAGGATT ATCCCAAGGA TATATCAAAC
TATGGCTTCC CCAGCAGCGT CCAAGCAATT GACGCAGCTG TTTTCTACAG AAGTAAAACA
TACTTCTTTG TAAATGACCA ATTCTGGAGA TATGATAACC AAAGACAATT CATGGAGCCA
GGTTATCCCA AAAGCATATC AGGTGCCTTT CCAGGAATAG AGAGTAAAGT TGATGCAGTT
TTCCAGCAAG AACATTTCTT CCATGTCTTC AGTGGACCAA GATATTACGC ATTTGATCTT
ATTGCTCAGA GAGTTACCAG AGTTGCAAGA GGCAATAAAT GGCTTAACTG TAGATATGGC
TGAAGCAAAA TCAAATGTGG CTGTATCCAC TTTCAGAATG TTGAAGGGAA GTTCAGCAAG
CATTTTCGTT ACATTGTGTC CTGCTTATAC TTTTCTCAAT ATTAAGTCAT TGTTTCCCAT
CACTGTATCC ATTCTACCTG TCCTCCGTGA AAATATGTTT GGAATATTCC ACTATTTGCA
GAGGCTTATT CAGTTCTTAC ACATTCCATC TTACATTAGT GATTCCATCA AAGAGAAGGA
AAGTAAGCCT TTTTGTCACC TCAATATTTA CTATTTCAAT ACTTACATAT CTGACTTCTA
GGATTTATTG TTATATTACT TGCCTATCTG ACTTCATACA TCCCTCAGTT TCTTAAAATG
TCCTATGTAT ATCTTCTACA TGCAATTTAG AACTAGATTT TGGTTAGAAG TAAGGATTAT
AAACAACCTA GACAGTACCC TTGGCCTTTA CAGAAAATAT GGTGCTGTTT TCTACCCTTG
GAAAGAAATG TAGATGATAT GTTTCGTGGG TTGAATTGTG TCCCCCATAA AAGATATGTT
GAAGTTCTAA CCCCAGGTAC CCATGAATGT GAGCTTACCA GGGTCTTTGC AGATGTAATT
AGTTAAGTTA AGGTGAGATC ACACTGAATT AGGGTGGGCT CTAAATCCAT TATGACTGTT
GTTCTTATAA GAAGAAGAGA GGCATAGTCA CCTAGGGGAG GAGGCCGTAT GAAGACAGAG
GCAGAGATTG GAGTGACGCA TCTCCAAGCC AAGGAATTCC AAGGACTGTA AGCCACCAGT
AGAAGCTTTG AAGAGGCAAG GAAGGATTCC CTCCAATAGC CTTCAAGTGT GACCCTGCTG
ACACCTGCAG AATTCGGACT TCTATCCTCC AAAACCGTGA GGGAATAAAT TTCCTTTGTT
TTAAGCCACC AACTTTGCAA TACTTTGTTA CAGCAACCCT AGACATGAGG TACTAGACAC
AGTACATCTA CACATATGAA AATGAATCAA CACAGAATGC AGAAGTAGAA CCCTTGCTAA
GGACTACTGG GCATCTTCCC AGGACAGCAG CCAAAAGAGA ACCACCACTT CCTCTCCTGC
CTCCTCCTTG CTCTCTCCTA GAGTCCAAAC CCAAATGGGC CAGTTGGATC TGATGTTCGT
CAGTTCTTTA CTTCTATTTC CTGGGGTACT CAGGAGGGCA CACACTATAG ATAACTTGGG
TTAGCTGCAT AAAATTCAAT GTCTCATTAA GTTGCATTAA ACTGAGCTTA GATGTGTAAG
TTTGCTAACG GATGGGTTTT TTTGTTAAGA ACTATAGGAT TTATGGGACC AAGTCTAGCG
AGTCCAGATA TCAAAATCAT TATAATGTTA TATTTGCTGT TATTAGAATA TAATATAGCT
TATTATACAA TAAATATGTA GACTGTAAAA TATATTTCTC ACTAGTACCT CCTATTTTCT
TTCTCTGTTG AAGTTTTTAA ATCCCACAGA TAATTAAATT GGCACCTTTA TGCTTGTTCA
AAAATTAAAA TAATCTATTA AATAAGTTCA AATTAAAGAT TTTTACTTCA AATGAC

Human MMP8 has the following amino acid sequence:

(SEQ ID NO: 44)
MFSLKTLPFL LLLHVQISKA FPVSSKEKNT KTVQDYLEKF YQLPSNQYQS TRKNGTNVIV
EKLKEMQRFF GLNVTGKPNE ETLDMMKKPR CGVPDSGGFM LTPGNPKWER TNLTYRIRNY
TPQLSEAEVE RAIKDAFELW SVASPLIFTR ISQGEADINI AFYQRDHGDN SPFDGPNGIL
AHAFQPGQGI GGDAHFDAEE TWTNTSANYN LFLVAAHEFG HSLGLAHSSD PGALMYPNYA
FRETSNYSLP QDDIDGIQAI YGLSSNPIQF TGPSTPKPCD PSLTFDAITT LRGEILFFKD
RYFWRRHPQL QRVEMNFISL FWPSLPTGIQ AAYEDFDRDL IFLFKGNQYW ALSGYDILQG
YPKDISNYGF PSSVQAIDAA VFYRSKTYFF VNDQFWRYDN QRQFMEPGYP KSISGAFPGI
ESKVDAVFQQ EHFFHVFSGP RYYAFDLIAQ RVTRVARGNK WLNCRYG

Developmental pluripotency associated 4 (DPPA4) is a protein encoded by the DPPA4 gene. Human DPPA4 has the following amino acid sequence:

(SEQ ID NO: 20)
AAGTGGGAGG AGACTTTGCA AATAGCAATC TTGGGGCAGG GGCCATTTTG GAAGCATGTT
GCGAGGCTCC GCTTCTTCTA CAAGTATGGA GAAGGCAAAA GGCAAGGAGT GGACCTCCAC
AGAGAAGTCG AGGGAAGAGG ATCAGCAGGC TTCTAATCAA CCAAATTCAA TTGCTTTGCC
AGGAACATCA GCAAAGAGAA CCAAAGAAAA AATGTCTATC AAAGGCAGTA AAGTGCTCTG
CCCTAAGAAA AAGGCAGAGC ACACTGACAA CCCCAGACCT CAGAAGAAGA TACCAATCCC
TCCATTACCT TCTAAACTGC CACCTGTTAA TCTGATTCAC CGGGACATTC TGCGGGCCTG
GTGCCAACAA TTGAAGCTGA GCTCCAAAGG CCAGAAATTG GATGCATATA AGCGCCTGTG
TGCCTTTGCC TACCCAAATC AAAAGGATTT TCCTAGCACA GCAAAAGAGG CCAAAATCCG
GAAATCATTG CAAAAAAAAT TAAAGGTGGA AAAGGGGGAA ACGTCCCTGC AAAGTTCTGA
GACACATCCT CCTGAAGTGG CTCTTCCTCC TGTGGGGGAG CCGCCTGCCC TGGAAAATTC
CACTGCTCTC CTTGAGGGAG TTAATACAGT TGTGGTGACA ACTTCTGCCC CAGAGGCTTT
GCTGGCCTCC TGGGCGAGAA TTTCAGCCAG GGCGAGGACA CCAGAGGCAG TGGAATCTCC
ACAAGAGGCC TCTGGTGTCA GGTGGTGTGT GGTCCATGGG AAAAGTCTCC CTGCAGACAC
AGATGGTTGG GTTCACCTGC AGTTTCATGC TGGTCAAGCC TGGGTTCCAG AAAAGCAAGA
AGGGAGAGTG AGTGCACTCT TCTTGCTTCC TGCCTCCAAT TTTCCACCCC CGCACCTTGA
AGACAATATG TTGTGCCCCA AATGTGTTCA CAGGAACAAG GTCTTAATAA AAAGCCTCCA
ATGGGAATAG AATATCAGGA AAAAGGCCAC ATCTATGGTA ATTAATGGCA GAAAAGCTGG
AGAGTTGGAT TCTGCGGTGC TGCTGACAGG TGAACTCTGG TCCTCTGCAC CTGTTTATGG
GCCATGCAGA CTGGTGGGGT GGCAGATGTT AGCCTAAGAC CCCTAGCAGT GCCTGTTGCT
TTGTGAGTGG AGATAGAGAC TCTTACATTT AAAAATGGAA AAACATTTCA CAAATTACCA
TAAATTGTAG TTAATATGTA GAAAAACTCA TTCATACTAC TTTTCTAAAA TAGACATGAC
TTCAGCAGCA GCTTTTTTTT GTTGTATTTT GAGACAGTGT CTCACTGTTG CCCAGGCTGG
AGTGCAGTGG TGCAATCTCA GTTCAGTGCA ATCTCCGCCT CCTGGGTTCA AATGATTCTC
CTGCCTCAGC CTCCTGAGTA GCTAGGTACA GGCACCTGCC ACCACACCCA GCTAATTTTT
TGTATTTTTA GTAGAGATGG GGTTTCACCA TGTTGGCCAG GTTGGTCTCA AACTCCTGGA
CTCAAGTGAT CACCCTCCTC AGCCTCCCAA AATGCTGGGA CTATGGGCAT GAGCCCCTGC
GCCTGACCTT CAACAGCTCT TTTAAGTGAG TTCTTCAGCT AAGCATTGTG ATGGACTTGA
GTAAAATGGT AGTTGGCTCT TGTGCTCAAT TTTCTCTTCC TCTGAACACT GACTACTTTA
GGAGCTGCTT CATTCCAATT GCAATTTCAT AAAACGTAAA GTATTTTAAG GCAAAGAAAG
GCTGTTAATT CCCTCCCTCC CCCAAACACA TGATTTTTAA TATTCTAAAC AATATTTTTC
AAAGTTCTCT TAATAACCTG AGATTTCTAT GGTTTGACTC CAGGATCAAA ACACAAGGGA
CTTTGTATTA TTTCACTTAT AATTGTTTTG TATATTTCTG GAGTTTAAAA TGTTTAAGGT
TGCTTCCCGC TCATAAATAC ATAATATATT GAATTTAAAA TGTGTTTATT AACCGATTCT
CCATAAATAA AAATAAGATG TGTATGTAAA ATAATTCATC TGTTGTATTT AGAGAACCAT
ATTCATTGCA TGCAAATTTT ATTGTTAGTG TTCTTAACTC AAGTAGGAGT AAACCAAAAA
GTGTGATTTT TCTTTTGTAT GACTCGTTTG TTCTTTATTA GTTGGTGGTA TGGGTTGGAT
CATTTGTTTT TAAAACTACT TAGGTATGAT TCACATACAA AAAGCTGCAC ATATTTAATG
TATCCTATTG TGTAATTAAT TTTTAATTTT TTTGTGTACT TCCTAAACTT ATAGTCCTGC
GAGTCTGGGA ACAGATCTGT TTTTCACTTA TCCTGATTTA ATGACAGTTT CCAACATTGT
TTTGTTATTA CAAGTAGGGG ATCTTTTTTT TTGCCCGTTT AATGAAGATA CTAAAAATAA
TGCACTGGAA GGAGTGGAAG AGTTGGAAAA TTTGTAACCA TCATAATACA GGTGTAATAG
GTTTGGGAAA GAATCCTCAA AAATGTTAAA GCAAGGGAGG AAAGTTTGTT GAGAAGCAAG
ATGTTCTTCT CTCCTGCCCG CCCCCGCCGT TGGTTGTTGG TGGTCAGAAT TATTGTGTAA
TAAATAATAG ACATTTTTTC TTATACTATG TGTATTGTTC CTTTTGTTTC CTTTTTAAAC
TTCTCCCCTG CTTTATTTGG ATGGGTCAAG TTTCTGTTCT GTTTCCTTCC TTTCTATTAA
TTTGGAAATG TCCTTGGCTT TACGATTCTG CTTGTAGATA CTTCCCCTGC TTCTAACACA
TTTCAATAAA CTTAAATTTC TCTATATACA AAATAAATTA ATAATTGGAG TCTACCAAAA
AAA

Human DPPA4 has the following amino acid sequence:

(SEQ ID NO: 45)
MLRGSASSTS MEKAKGKEWT STEKSREEDQ QASNQPNSIA LPGTSAKRTK EKMSIKGSKV
LCPKKKAEHT DNPRPQKKIP IPPLPSKLPP VNLIHRDILR AWCQQLKLSS KGQKLDAYKR
LCAFAYPNQK DFPSTAKEAK IRKSLQKKLK VEKGETSLQS SETHPPEVAL PPVGEPPALE
NSTALLEGVN TVVVTTSAPE ALLASWARIS ARARTPEAVE SPOEASGVRW CVVHGKSLPA
DTDGWVHLQF HAGQAWVPEK QEGRVSALFL LPASNFPPPH LEDNMLCPKC VHRNKVLIKS
LQWE

Endothelial cell-specific molecule 2 (ECSM2) is a protein encoded by the ECSM2 gene. Human ECSM2 has the following amino acid sequence:

TCTCTTCTCC ACTATGGACA GAGCCTCCAC TGAGCTGCTG CCTGCCCGCC ACATACCCAG
CTGACATGGG CACCGCAGGA GCCATGCAGC TGTGCTGGGT GATCCTGGGC TTCCTCCTGT
TCCGAGGCCA CAACTCCCAG CCCACAATGA CCCAGACCTC TAGCTCTCAG GGAGGCCTTG
GCGGTCTAAG TCTGACCACA GAGCCAGTTT CTTCCAACCC AGGATACATC CCTTCCTCAG
AGGCTAACAG GCCAAGCCAT CTGTCCAGCA CTGGTACCCC AGGCGCAGGT GTCCCCAGCA
GAGGAAGAGA CGGAGGCACA AGCAGAGACA CATTTCAAAC TGTTCCCCCC AATTCAACCA
CCATGAGCCT GAGCATGAGG GAAGATGCGA CCATCCTGCC CAGCCCCACG TCAGAGACTG
TGCTCACTGT GGCTGCATTT GGTGTTATCA GCTTCATTGT CATCCTGGTG GTTGTGGTGA
TCATCCTAGT TGGTGTGGTC AGCCTGAGGT TCAAGTGTCG GAAGAGCAAG GAGTCTGAAG
ATCCCCAGAA ACCTGGGAGT TCAGGGCTGT CTGAAAGCTG CTCCACAGCC AATGGAGAGA
AAGACAGCAT CACCCTTATC TCCATGAAGA ACATCAACAT GAATAATGGC AAACAAAGTC
TCTCAGCAGA GAAGGTTCTT TAAAAGCAAC TTTGGGTCCC CATGAGTCCA AGGATGATGC
AGCTGCCCTG TGACTACAAG GAGGAAGAGA TGGAATTAGT AGAGGCAATG AACCACATGT
AAATTATTTT ATTGTTTCAT GTCTGCTTCT AGATCTAAAG GACACTAGCA TTGCCCCAGA
TCTGGGAGCA AGCTACCAAC AGGGGAGACT CTTTCCTGTA TGGACAGCTG CTGTGGAAAT
ACTGCCTGCT TCTCCCACCT CCTCAGAGCC ACAGGAAAGA GGAGGTGACA GAGAGAGAGC
AAGGAAAGTG ATGAGGTGGA TTGATACTTT CTACTTTGCA TTAAAATTAT TTTCTAGCCT

Human ECSM2 has the following amino acid sequence:

(SEQ ID NO: 46)
MGTAGAMQLC WVILGFLLFR GHNSQPTMTQ TSSSQGGLGG LSLTTEPVSS NPGYIPSSEA
NRPSHLSSTG TPGAGVPSSG RDGGTSRDTF QTVPPNSTTM SLSMREDATI LPSPTSETVL
TVAAFGVISF IVILVVVVII INGVVSLREK CRKSKESEDP QKPGSSGLSE SCSTANGEKD
SITLISMKNI NMNNGKQSLS AEKVL

The disclosed biomarker can be an expression product of a gene having the following nucleic acid sequence (GenBank Accession No. AA393032.1):

(SEQ ID NO: 13)
GCTTTTTAAA TGACCCAGGC GTGTGTAATA ATATAATGAA TAACCATAGA GCAGTGCCTT
TAAATTAGCT ATAGGAAGGA AATAGTCTTT TCAAGTTTCT GAACAATATA TTTCTCTTAG
TTGGCACCTC ACAAATACTA GATCATGTCA GACGCTGCTG GTTAATAGCT GCAGGAAGGC
ATGTTGTGCA GTGGATATTG CTCATGGAAG TGTGTGAAAT CATAGTAAGC TTTGTTCTCC
CTGCTAAGAC TTGCTATGTA TATTTCCATC ATTGTTTCAT GTAAACTGAA CCATTGTGGT
AAACTTTTGG AGTTGATATG GAATCACTTT AATGCTGTTT TCACAAATAA AAGTT

The disclosed biomarker can therefore be an mRNA or protein encoded by SEQ ID NO:13.

The disclosed biomarker can be the gene having the following nucleic acid sequence (GenBank mRNA ID: AK026379):

(SEQ ID NO: 14)
TATAAACAAC ATTCAAATAA CCTTGGACCT TGGTGAAATG ACTTGTGGTG GCCAGAATGG
TGCAACAAGA TGTTATTTGC AAGTTTTGTT AAGACACAAA TATCTCAGAT ACTAATAATG
AGAATAAAGA CTGTTGAATA TGAAATTAAA GCCAAGCAAT AATGTGCCAA AAAGAGGCAG
TTATACCAGC AAATGCATCT ATTATGGGCA CACCATTATA TAATGATGGT TTGCTTTATG
AAGACTGACT GTAACCCACA GGATAAAATA AGCAAAGGCA TAGTTTCTGC TTTCTTCCTG
GAAAAACTTG TTTAGAAGCT TCATAAAGAG GTACAGCACT AATGAGCATT AGTCAGGATA
CAGTTGGCAT CTATGTTTTT ATGTGAGCCC AGAGGGAAGA GGAGCCACTC AAAGTCTTGC
TGGCTTAAAA CTCAAGACAG CTGCAACCAG AAGTTTTGTT GAAATGGAGA CTTTAAACTT
ATGGTAATTA CTCTTTCTGG ACACTAGCAT GTAGAAAGCA ATTCAGTTAA CTCTGCCCAG
AGGATTACCA GCTTTAGCTG TGAAAAAATG GGCTCCCGGA TGTAAAATCA CTAAAACATG
AGATCTTGTA TCCAAAGAGG CTTCAAATGA TGCCTTACAG AAAACGATGC TCCAGATGGG
CACTTCTAAA TGCTAACTCT TCATCAAGTA TCTTTCTGGA TTCAAGCTCA AAATTAATTG
GCTGCAAAAT AGTAGGAATA AAAATCACAT ATTTTACACT TTAGAAAAGG ATATTGATGA
TCAACCTGCA TGGTGATAAT TATGATGAGA TACCCCAGTG ATTTAATGAT GTTAGAAAGA
ATTAAATGGG AGAGAATTGC TAACAGCTTT CTTGATCTCT TAACTATGGA GATGTCATTC
ATTTATTTCT GGGGTGAAAA TTATAGCTTG CTTTTTGACA TTGCTGCTAG TATTGTTCTT
TGTTGCTTTA AAAATTGTCT CTCTTTAGAA AAACTCTTGA GCAGTTAAAC AGTTTTTTTT
CTGATTCATA TCATTGCTTT TAATAACATG TAAAGGCTGT GTGTAGAGCA AACTATATAA
AATGAGTAGA AAGGGCTTAC TCATGTTAAT TGGCATCCTT GATGATTTTA GTTGAGATTC
CTTAACATTT ATTTTAGATC ACATCTTTAC GTAACTTATT TTTCCTAATG TTTTCCATCG
TGTCTTAAAA TGATGCTGGT ATATCAGGAG ATTGCAGTAT TATAGTCATA CTCCCCAATC
CCTAGAGGAG AGGAAAGACT AATTCTTGTT TTAAGGGCCC CTGGAGATAC CTTTTATTAA
GGTTGAAAAA GGTCAACACA GCCTGAAAAT AAGAAAAATA TATACTAGCA ATTACTAATT
TTCTAAATGT GTGTATCTCT GCTGTACTAA TGTGTGAACA ATATGTCGTG CATAATACTG
TAGCTGGTCG TGGTATGTCA ATACATTCTG TGAGTGTGTA CAGTCTGAGT GATCAGTTTT
CTATTTTTAT GTGTAAAAAA AATAACTTGT CGTATCCCAT TTAAAGGCCA ATTTCTGTAT
TCAGGCAGGC ATATGTACAT ACATGAATAA AGCCAACAAA AGTGTGCACA TGTAAAAAAA
AAAAAAAAAA

The disclosed biomarker can be protein encoded by SEQ ID NO:14.

The disclosed biomarker can be the gene having the following nucleic acid sequence (GenBank mRNA ID: AI271427):

(SEQ ID NO: 18)
TTTTTTTTTT TTTTTTTTAA CAGGAGTTAT TTCTGATTTT ATTTATAATA TAAAAATGTT
CAAGTGTCAA CAGTCAGGTG TTCAGACATT TCAGGACAGG ATTCCCATCT GTTTCTGTTT
GGGATTTTTT TTTTTTTTTT AAACAATTAC CTTTTTGACA AATTAGCAGT GGACCCAGTT
TTTGGGGGTG GGAGGGCAGG ACTGGAGACG AGTGGATGTC ATAGGTGGGT TGGGGGCTAG
GAGGCAGCCT GTGAGAAGGA AATGGTGTTA CTTTATTGCT AAAAGGGGAA TACACTGTCG
AGTGGCTCTT CTCGGTCCCA GCGTGACCAT GCATCCAATC TAAAGAATCT GAAATGCAAA
GGACATGCAG GTGTAAAATA GAAAAGACGA CCTGTAAACG AAGGTGCTGC AAAGGACGGA
GGGGCGTCCT GG

The disclosed biomarker can be protein encoded by SEQ ID NO:18.

More than one biological marker, for example 2, 3, 4, 5, 6, 7, 8, 9, 10, 11, 12, 13, 14, 15, 16, 17, 18, 19, 20, or 21 nucleic markers, and/or 2, 3, 4, 5, 6, 7, 8, 9, 10, 11, 12, 13, 14, 15, 16, 17, 18, 19, 20, 21, 22, 23, 24, or 25 polypeptide markers, may detected or determined.

B. Protein Detection of Biomarkers

In some embodiments, trichogenic dermal cells, such as DP cells and DS cells, can be detected, identified, and enriched in assays that detect protein expression. Trichogenic dermal cells, such as DP cells and DS cells, can be detected, identified, and enriched using a variety of conventional techniques including, but not limited to immunological, spectrophotometric, fluorometric, and colormetric assays.

In some embodiment, trichogenic dermal cells, such as DP cells and DS cells, are detected using antibodies that specifically bind the one or more biomarkers disclosed herein. Serglycin (SRGN), Src-like-adaptor—encoded polypeptide 3 (SLA), Thrombomodulin (THBD), Runt-related transcription factor 2 (RUNX2), Runt-related transcription factor 3 (RUNX3), Protocadherin 17 (PCDH17), Lymphocyte antigen 75 (LY75), Placental Growth Factor (PGF), Amyloid beta (A4) precursor protein-binding, family A, member 2 (APBA2), Prostaglandin E synthase (PTGES), myosin IF (MYO1F), G protein-coupled receptor 84 (GPR84), Transcription elongation factor A (SII)-like 2 (TCEAL2), Collagen, type XXIII, alpha 1 (COL23A1), ST8 alpha-N-acetyl-neuraminide alpha-2,8-sialyltransferase 4 (ST8SIA4), Matrix metallopeptidase 8 (MMP8), Developmental pluripotency associated 4 (DPPA4), and Endothelial cell-specific molecule 2 (ECSM2) antibodies are commercially available, for example, from Abeam (Cambridge, Mass.), R&D Systems (Minneapolis, Minn.), Santa Cruz Biotechnology (Santa Cruz, Calif.), and Sigma-Aldrich (St. Louis, Mo.).

The antibody can be labelled with a detectable label such as fluorescent labels, chemiluminescent labels, chromophores, antibodies, enzymatic markers, radioactive isotopes, affinity tags and photoreactive groups.

C. Nucleic Acid Detection of Biomarkers

In some embodiments, trichogenic dermal cells, such as DP cells and DS cells, can be detected, identified, and enriched in assays that detect nucleic acid expression, such as mRNA expression. A number of widely used procedures exist for detecting and determining the abundance of a particular mRNA in a total or poly(A) RNA sample. For example, specific mRNAs can be detected using Northern blot analysis, nuclease protection assays (NPA), in situ hybridization, or reverse transcription-polymerase chain reaction (RT-PCR), and microarray analysis.

In theory, each of these techniques can be used to detect specific RNAs and to precisely determine their expression level. In general, Northern analysis is the only method that provides information about transcript size, whereas NPAs is one way to simultaneously examine multiple messages. In situ hybridization is used to localize expression of a particular gene within a tissue or cell type, and RT-PCR is the most sensitive method for detecting and quantitating gene expression.

Relative quantitative RT-PCR involves amplifying an internal control simultaneously with the gene of interest. The internal control is used to normalize the samples. Once normalized, direct comparisons of relative abundance of a specific mRNA can be made across the samples. It is crucial to choose an internal control with a constant level of expression across all experimental samples (i.e., not affected by experimental treatment). Commonly used internal controls (e.g., GAPDH, β-actin, cyclophilin) often vary in expression and, therefore, may not be appropriate internal controls. Additionally, most common internal controls are expressed at much higher levels than the mRNA being studied. For relative RT-PCR results to be meaningful, all products of the PCR reaction must be analyzed in the linear range of amplification. This becomes difficult for transcripts of widely different levels of abundance.

Competitive RT-PCR is used for absolute quantitation. This technique involves designing, synthesizing, and accurately quantitating a competitor RNA that can be distinguished from the endogenous target by a small difference in size or sequence. Known amounts of the competitor RNA are added to experimental samples and RT-PCR is performed. Signals from the endogenous target are compared with signals from the competitor to determine the amount of target present in the sample.

Method for detecting nucleic acids, such as RNA, generally involve the use of an oligonucleotide primer or probe that hybridizes to the target nucleic acid. Therefore, oligonucleotides are also provided for use as primer or probes for the detection of one or more of the disclosed biological markers. The disclosed oligonucleotide can be a fragment of one or more of the disclosed nucleic acid biomarkers, such as those set forth in SEQ ID NO:1, 2, 3, 4, 5, 6, 7, 8, 9, 10, 11, 12, 13, 14, 15, 16, 17, 18, 19, 20, or 21, or the complement thereof. For example, the oligonucleotide can include at least 6, 7, 8, 9, 10, 11, 12, 13, 14, 15, 16, 17, 18, 19, 20, 25, 30, 35, 40, 45, 50, 60, 70, 80, 90, 100 consecutive nucleic acids set forth in SEQ ID NO: 1, 2, 3, 4, 5, 6, 7, 8, 9, 10, 11, 12, 13, 14, 15, 16, 17, 18, 19, 20, or 21, or the complement thereof. Moreover, the oligonucleotide can include at least 6, 7, 8, 9, 10, 11, 12, 13, 14, 15, 16, 17, 18, 19, 20, 25, 30, 35, 40, 45, 50, 60, 70, 80, 90, 100 consecutive nucleic acids of a nucleic acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NOs:1, 2, 3, 4, 5, 6, 7, 8, 9, 10, 11, 12, 13, 14, 15, 16, 17, 18, 19, 20, or 21, or the complement thereof. Therefore, the oligonucleotide can include at least 6, 7, 8, 9, 10, 11, 12, 13, 14, 15, 16, 17, 18, 19, 20, 25, 30, 35, 40, 45, 50, 60, 70, 80, 90, 100 consecutive nucleic acids of a nucleic acid sequence that hybridizes under stringent conditions to an oligonucleotide consisting of the nucleic acid sequence SEQ ID NOs:1, 2, 3, 4, 5, 6, 7, 8, 9, 10, 11, 12, 13, 14, 15, 16, 17, 18, 19, 20, or 21, or the complement thereof.

Arrays, such as microarrays, that contain 1, 2, 3, 4, 5, 6, 7, 8, 9, 10, 11, 12, 13, 14, 15, 16, 17, 18, 19, 20, or 21 of the disclosed oligonucleotides are also provided. These arrays can be used to detect multiple biomarkers simulataneously.

D. Cell Populations Enriched for DP Cells and DS Cells

Populations of skin cells enriched for trichogenic dermal cells, such as DP cells and DS cells, are also provided. The population of skin cells can be enriched for cells expressing the one or more biomarkers disclosed herein by at least about 5%, 10%, 15%, 20%, 25%, 30%, 35%, or 40%. The initial skin cell population can be obtained from a mammalian subject, preferably from a human. The initial skin cell population can be obtained from a cell culture containing DP and/or DS cells. The initial skin cell population can be heterogeneous in that it can contain, for example, DP cells, DS cells, fibroblasts, melanocytes, and keratinocytes. The initial skin cell population can be heterogeneous in that it can contain, for example, both trichogenic and non-trichogenic dermal cells. In a preferred embodiment, the enriched trichogenic dermal cell population is homogeneous in that it contains only trichogenic decal cells, such as trichogenic DP cells and/or DS cells.

Methods for enriching cell populations based on protein expression are known in the art and include, but are not limited to, flow cytometry and immunological separation techniques. A preferred technique for enriching DP cells and DS cells uses commercially available reagents such as CELLection™ Biotin Binder Kit from Invitrogen. Generally, biotinylated antibodies to the one or more disclosed biomarkers are added to a suspension of skin cells. Next, streptavidin conjugated beads are added to the suspension and bind to biotinylated antibody bound to cells positive for the one or more biomarkers. A magnet is then used to separate the DP cells and/or DS cells from the other skin cells and thereby form two populations of cells. One population is enriched with DP cells and/or DS cells and the other population has a significantly reduced number of DP cells and/or DS cells.

A skin cell population of enriched trichogenic dermal cells, such as DP cells and/or DS cells, combined with epidermal cells is also provided. The epidermal:dermal can be present in the suspension in a ratio of about 0:1, 1:1, 1:2 and 1:10. The suspension of trichogenic dermal cells and epidermal cells can further include additional cell types, such as melanocytes.

Aggregates of enriched trichogenic dermal cells and epidermal cells are also provided. The epidermal:dermal can be present in the aggregates in a ratio of about 0:1, 1:1, 1:2 and 1:10. The aggregate of trichogenic dermal cells and epidermal cells can further include additional cell types, such as melanocytes. The cells can be aggregated by suspension growth in a non-stick tissue culture dish, or by centrifugation of the cultured cells. In certain embodiments, a suitable aggregation enhancing substance may be added to the cells prior to, or at the time of, implantation. Suitable aggregation enhancing substances include, but are not limited to, glycoproteins such as fibronection or glycosaminoglycans, dermatan sulfate, chondroitin sulfates, proteoglycans, heparin sulfate and collagen.

C. Kits

Kits are also provided that include a container containing antibodies that selectively bind the one or more biomarkers disclosed herein for use in detecting, identifying or enriching trichogenic dermal cells, such as DP cells and/or DS cells.

Kits are also provided that include a container containing oligonucleotides that hybridize to the one or more nucleic acid biomarkers disclosed herein for detection of trichogenic dermal cells, such as DP cells and/or DS cells.

The kit can also include reagents for detecting the nucleic acid biomarkers. Alternatively, the kits can contain antibodies that bind to the protein biomarkers. The antibodies are preferably labeled with a detectable label.

### III. Methods of Identifying and Isolating DP and/or DS Cells

The one or more biomarkers disclosed herein can be used to identify cells that have the ability to induce hair follicle formation, i.e., are trichogenic. Therefore, the one or more biomarkers disclosed herein can be used to identify trichogenic dermal cells, such as DP cells and/or DS cells. Generally, cells are harvested from an animal, for example a mouse or human. The cells can be autologous or allogenic. Tissue, preferably scalp tissue, is obtained from a subject, such as a human fetus, child, or adult, and processed to obtain dissociated cells using techniques known in the art. The cells can be a mixed population of cells containing DP and/or DS cells and other skin cells, such as fibroblasts and keratinocytes. In some embodiments the mixed population of cells includes both dermal and epidermal cells. In some embodiments the mixed population of cells includes both trichogenic and non-trichogenic dermal cells.

Trichogenic dermal cells, such as DP and/or DS cells, in a mixed population of skin cells can be identified by assaying the cells for expression of one or more biomarkers disclosed herein. For example, the biomarker can be Serglycin (SRGN), Src-like-adaptor—encoded polypeptide 3 (SLA), Thrombomodulin (THBD), Runt-related transcription factor 2 (RUNX2), Runt-related transcription factor 3 (RUNX3), Protocadherin 17 (PCDH17), Lymphocyte antigen 75 (LY75), Placental Growth Factor (PGF), Amyloid beta (A4) precursor protein-binding, family A, member 2 (APBA2), Prostaglandin E synthase (PTGES), myosin IF (MYO1F), G protein-coupled receptor 84 (GPR84), Transcription elongation factor A (SII)-like 2 (TCEAL2), Collagen, type XXIII, alpha 1 (COL23A1), ST8 alpha-N-acetyl-neuraminide alpha-2,8-sialyltransferase 4 (ST8SIA4), Matrix metallopeptidase 8 (MMP8), Developmental pluripotency associated 4 (DPPA4), Endothelial cell-specific molecule 2 (ECSM2), or a combination thereof.

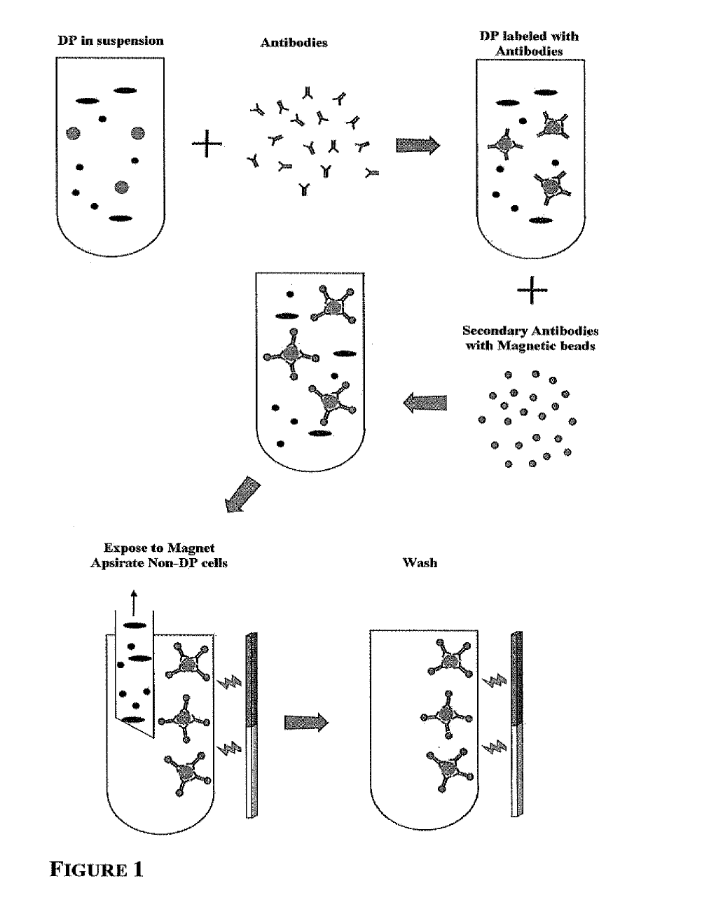

In one embodiment a population of cells enriched for expression of one or more trichogenic biomarkers is obtained by cell sorting using CELLection™ Biotin Binder Kit. Both direct and indirect methods can be employed. Basically, a biotinylated anti-biomarker antibody is added to the cell sample at 1 μg per 1 million cells (indirect method) or added to streptavidin coated beads at 2 μg/25 ul beads (direct method) and incubated at 4° C. overnight. The streptavidin coated beads can be moved using a magnet. Next, the streptavidin coated beads and cell sample are mixed together so the biomarker positive cells attach to the streptavidin coated beads through the biotinylated anti-biomarker antibody. The bead-bound-cells are then separated from other cells by a magnet. The biomarker positive cells are then released from the magnetic beads. The beads are then removed using magnets. See FIG. 1 for a schematic illustration of a method of enriching cells using antibodies specific for a trichogenic biomarker.

In another embodiment, biomarker expression is detected by Guava Analyzer. Briefly, cells are first incubated with a Phycoerythrin conjugated anti-biomarker antibody at 4° C. for half an hour. Then the cells are washed two times with Dulbecco's Phosphate Buffered Saline (DPBS) with bovine serum albumin (0.1% BSA) plus antibiotic (clindamycin, actinomycin, streptomycin). Biomarker expression level is measured by GUAVA Analyzer.

The method can in some embodiments, involve detection in the cell of the nucleic acid encoding one or more nucleic acid biomarkers disclosed herein. For example, the biomarker can be SRGN, SLA, THBD, RUNX2, RUNX3, PCDH17, LY75, PGF, APBA2, PTGES, MYO1F, GPR84, TCEAL2, COL23A1, ST8SIA4, MMP8, DPPA4, ECSM2, or a combination thereof. Methods for identifying nucleic acid or protein biomarkers are known in the art. Quantitative Real-Time PCR, flow cytometry and immunological techniques are preferred.

### IV. Methods of Using Enriched DP and DS Cells

Populations of trichogenic dermal cells, such as DP cells and/or DS cells, can be used to replace, augment, or restore hair. The disclosed trichogenic dermal cells selected or enriched for expression of one or more biomarkers represent an improvement over prior art methods using dermal cells not selected or enriched for trichogenicity. Therefore, the disclosed enriched trichogenic dermal cells can be injected subcutaneously or intradermally to induce the formation of new hair follicles. The new hair follicles generate new hair shafts. Thus, the enriched trichogenic dermal cells can replace or augment existing hair follicles by inducing the formation of new or additional hair follicles that generate new hair shafts. Alternatively, the populations of enriched trichogenic dermal cells, such as DP cells and/or DS cells, can be injected subcutaneously or intradermally to induce existing hair follicles to generate new terminal hair. For example, a population of enriched trichogenic dermal cells can be injected adjacent to one or more existing hair follicles that produce vellus hair. The enriched trichogenic dermal cells then induce the vellus hair follicle to produce terminal hair. These methods for using enriched trichogenic dermal cell populations are described in more detail below.

A. Hair Follicle Induction

Enriched trichogenic dermal cells, such as DP cells and/or DS cells that express one or more of the disclosed biomarkers, can be used to generate new hair follicles in a subject. Typically, the enriched dermal cell population is autologous or allogenic.

Subjects to be transplanted with enriched trichogenic dermal cells include any subject that has an insufficient amount of hair or an insufficient rate of hair growth at a site or region of skin. The enriched trichogenic dermal cells can be used to treat hair loss resulting from androgenetic alopecia, wounding, trauma, scarring, telogen effluvium, genetic pattern baldness or with hormonal disorders that decrease hair growth or cause loss of hair. Subjects may have these conditions or be at risk for the development of these conditions, based on genetic, behavioral or environmental predispositions or other factors. Other suitable subjects include those that have received a treatment, such as chemotherapy or radiation, that causes a decrease in hair growth or a loss of hair. The enriched trichogenic dermal cells can also be used to treat scalp or hair trauma, structural hair shaft abnormalities, or a surgical procedure, such as a skin graft, which results in an area of skin in need of hair follicles.

In certain embodiments, the enriched population of trichogenic dermal cells, such as DP cells and/or DS cells that express one or more of the disclosed biomarkers, are combined with epidermal cells prior to implantation in a subject. Preferred locations for implantation include body skin including, but not limited to the subject's scalp or face. In one embodiment enriched trichogenic dermal cells are injected alone.

In a preferred embodiment, enriched trichogenic dermal cells and epidermal cells are cultured and expanded prior to implantation to obtain a sufficiently large number of cells suitable for implantation at multiple sites of a host to form new hair follicles. The cells are cultured in a manner that maintains the trichogenic activity of the dermal cells. Methods for culturing dissociated dermal and epidermal cells are known in the art. Dermal cells may be cultured separately from epidermal cells or may be co-cultured with epidermal cells. Exemplary methods for culturing dermal cells are provided in Rob, et al., Physiol. Genomics, 19:207-17 (2004) and McElwee, et al., Jour. Invest. Dermatol., 121(6):1267-75 (2003).

Suitable cell culture media include commercially available media, such as Dulbecco's Modified Eagle Medium/Nutrient Mixture F-12 (DMEM/F-12), RPMI-1640 and Ham's F10 (Sigma). The medium may be supplemented as appropriate with serum (such as fetal bovine serum, calf serum or horse serum), hormones or other growth factors (such as insulin, epidermal growth factor, Wnt polypeptides, or transferrin), ions (such as sodium, chloride or calcium), buffers (such as HEPES), nucleosides or trace elements.

The cells that are implanted into the subject may be autologous, allogenic or xenogenic. In one embodiment, enriched trichogenic dermal cells, such as DP cells and/or DS cells that express one or more of the disclosed biomarkers, and epidermal cells are obtained from skin sections from a single allogenic donor. In another embodiment, trichogenic dermal cells and epidermal cells are obtained from skin sections from more than one donor. For example, enriched trichogenic dermal cells may be derived from one donor and epidermal cells from another donor. In a preferred embodiment, the cells that are implanted are autologous.

Enriched trichogenic dermal cells and epidermal cells can be combined at an appropriate ratio prior to implanting into the subject. The epidermal:dermal ratio would range from 0:1, 1:1, 1:2 and 1:10. Dermal cells and epidermal cells can be further combined with additional cell types, such as melanocytes, prior to implantation. The enriched trichogenic dermal cells and epidermal cells to be implanted can be subjected to physical and/or biochemical aggregation prior to implanting to induce and/or maintain aggregation of the cells within the transplantation site. For example, the cells can be aggregated by suspension growth in a non-stick tissue culture dish, or by centrifugation of the cultured cells. In certain embodiments, a suitable aggregation enhancing substance may be added to the cells prior to, or at the time of, implantation. Suitable aggregation enhancing substances include, but are not limited to, glycoproteins such as fibronection or glycosaminoglycans, dermatan sulfate, chondroitin sulfates, proteoglycans, heparin sulfate and collagen.

The enriched trichogenic dermal cells, such as DP cells and/or DS cells that express one or more of the disclosed biomarkers, may be implanted into a subject using routine methods known in the art. Various routes of administration and various sites can be used. For example, the cells can be introduced directly between the dermis and the epidermis of the outer skin layer at a treatment site. This can be achieved by raising a blister on the skin at the treatment site and introducing the cells into fluid of the blister. The cells may also be introduced into a suitable incision extending through the epidermis down into the dermis. The incision can be made using routine techniques, for example, using a scalpel or hypodermic needle. The incision may be filled with cells generally up to a level in direct proximity to the epidermis at either side of the incision. In a preferred embodiment, the cells are delivered using a device as described in US Patent Application Publication No. 2007/0233038 to Pruitt, et al.

The dosage of cells to be injected is typically between about one million to about four million cells per square cm.

In another embodiment, a plurality of small recipient sites, for example, 10, 50, 100, 500 or 1000 or more is formed in the skin into which the cells are transplanted. Each perforation can be filled with a plurality of cells. The size and depth of the perforations can be varied. The perforations in the skin can be formed by routine techniques and can include the use of a skin-cutting instrument, e.g., a scalpel or a hypodermic needle or a laser (e.g., a low power laser). Alternatively, a multiple-perforation apparatus can be used having a plurality of spaced cutting edges formed and arranged for simultaneously forming a plurality of spaced perforations in the skin. The cells can be introduced simultaneously into a plurality of perforations in the skin.

The number of cells introduced into each perforation can vary depending on various factors, for example, the size and depth of the opening and the overall viability and trichogenic activity of the cells. In one embodiment about 50,000 to about 2,000,000 cells are delivered per injection. The cell concentration can be about 5,000 to about 1,000,000 cells/μl, typically about 50,000 cells/μl to about 75,000 cells/μl. A representative volume of cells delivered per injection is about 1 to about 10 μl, preferably about 4 μl. In one embodiment, 1 to 100 injections per cm², typically 1 to 30 injections per cm² are made in the skin, preferably the scalp.

The epidermal cells, dermal cells, or combinations thereof may be combined with a pharmacologically and/or physiologically suitable carrier such as saline solution, phosphate buffered saline solution, Dulbecco's Phosphate Buffered Saline (“DPBS”), DMEM, D-MEM-F-12 or HYPOTHERMOSOL-FRS from BioLifeSolutions (Bothell, Wash.) or a preservation solution such as a solution including, but not limited to, distilled water or deionized water, mixed with potassium lactobionate, potassium phosphate, raffinose, adenosine, allopurinol, pentastarch prostaglandin El, nitroglycerin, and/or N-acetylcysteine into the solution. Typically, the injected cells are suspended in cell culture media used to culture the cells. The preservation solution employed may be similar to standard organ and biological tissue preservation aqueous cold storage solutions such as HYPOTHERMOSOL-FRS from BioLifeSolutions (Bothell, Wash.).

The cells and the carrier may be combined to form a suspension suitable for injection. Each injection will typically include about 1.0 μl to about 10 it of composition or suspension. The injection may be performed with any suitable needle, syringe or other instrument. A 25 gauge needle attached to a syringe loaded with the composition or suspension may be used. Alternatively, a hubless insulin syringe may also be used to inject the composition into skin of a mammal. The suspension may also be delivered by other suitable methods, such as spreading the composition or suspension over superficial cuts of the skin or pipetting the composition or suspension into an artificially created wound.

The use of dermal and/or epidermal cells derived from an allogenic source may require administration of an immunosuppressant, alteration of histocompatibility antigens, or use of a barrier device to prevent rejection of the implanted cells. Cells can be administered alone or in conjunction with a barrier or agent for inhibiting or reducing immune responses against the transplanted cells in a recipient subject. For example, an immunosuppressive agent can be administered to a subject to inhibit or interfere with normal response in the subject. The immunosuppressive agent can be an immunosuppressive drug that inhibits T cell/or B cell activity in the subject or an antibody to t-cells. Suitable immunosuppressive drugs are commercially available. An immunosuppressive agent can be administered to a subject at a dosage sufficient to achieve the desired therapeutic effect (e.g., inhibition of rejection of the cells).

In some embodiments, the subject is treated, topically and/or systematically, with a hair growth promoting substance before, at the same time as, and/or after the transplantation of cells to enhance hair growth. Suitable hair growth promoting substances can include, e.g., minoxidil, cyclosporin, and natural or synthetic steroid hormones and their enhancers and antagonists, e.g., anti-androgens, all of which are commercially available.

B. Terminal Hair Induction

Another embodiment provides a method for inducing vellus hair to become terminal hair. Vellus hair is the fine, non-pigmented hair (peach fuzz) that covers the body of children and adults. Terminal hair is developed hair, which is generally longer, coarser, thicker and darker than the shorter and finer vellus hair. The growth of vellus hair is not affected by hormones; whereas, the growth of terminal hair is affected by hormones. Vellus hair is also present in male pattern baldness.

In one embodiment a population of skin cells enriched for trichogenic dermal cells, such as DP cells and/or DS cells that express one or more of the disclosed biomarkers, are injected into a skin as described above. The enriched trichogenic dermal cells are obtained as described above and are typically autologous or allogenic cells. The cells are injected adjacent to vellus hair or vellus hair follicles. Multiple injections of enriched trichogenic dermal cells can be delivered to an area of skin containing vellus hair to induce as many vellus hair follicles as possible to become terminal hair follicles. It will be appreciated that the number of injections and volume of cells to be injected can be routinely determined by one of skill in the art.

In another embodiment, enriched trichogenic dermal cells are injected into skin in an amount effective to induce formation of hair follicles and to induce vellus hair follicles to become terminal hair follicles. In one embodiment, the number of cells injected is effective to induce hair follicle formation in a period of about two weeks to about twelve weeks. In another embodiment, the injected cells induce terminal hair formation from vellus hair in a period of about two weeks to about twelve weeks.

### EXAMPLES

### Example 1

### Identification of Hair Induction-Capable and Hair Induction-Incapable Gene Markers

Identification of the hair induction-capable and hair induction-incapable gene markers was done in two parts. First, genes were identified that are expressed in DP/DS cells and not in fibroblasts or keratinocytes. Gene expression was compared in these cell types using microarray analysis. Second, selected genes that are expressed in DP/DS cells were further screened to compare RNA expression between hair induction-capable, hair induction incapable DP/DS cells. Gene expression was compared in these cell types using real-time quantitative PCR (qPCR) analysis.

Materials and Methods

Methodology Used in the Identification of Markers

Total RNA was prepared from 9 cell culture samples and 3 freshly isolated tissue samples. The 12 samples fell into the groups below:

Group 1: cultured human dermal fibroblasts (HDF) from 3 independent donors;

Group 2: cultured human keratinocytes (HK) from 3 independent donors;

Group 3: cultured dermal papilla cells (DP cells) from 3 independent donors; and

Group 4: freshly isolated dermal papillae (DPfr) from 3 independent pools of donors.

RNA extraction, purification, analysis, labelling, profiling on microarrays and primary microarray data analysis was performed by ALMAC Diagnostics (Durham, N.C., USA) in accordance with Minimum Information About a Microarray Experiment (MIAME) standards (see Brazma et al., 2001, Nature Genetics 29: 365-371 and MGED Society website http://www.mged.org/Workgroups/MIAME/miame_{—}2.0.html).

The 12 RNA samples were assessed for quality by spectrophotometry and Agilent Bioanalyzer analysis. High quality RNA samples were used to generate labelled nucleic acid samples that were profiled on Affymetrix Human Genome U133 Plus2 Arrays. Nucleic acid preparations were amplified using the NuGEN™ Ovation™ RNA Amplification System V2 (see http://www.nugeninc.com/nugen/index.cfm/products/amplification-systems/ovation-amp-v2/?keywords=3100-12). The amplified cDNA was then labeled using the FL-Ovation™ cDNA Biotin Module V2 (see http://www.nugeninc.com/nugen/index.cfm/products/target-prep-modules/fl-ovation-biotin-v21/?keywords=4200-12).

The resultant labelled cDNA was hybridised onto Affymetrix GeneChip® arrays. Following the hybridisation, the array was washed and stained using a GeneChip® Fluidics Station 450 using the appropriate fluidics script, before being inserted into the Affymetrix autoloader carousel and scanned using the GeneChip® Scanner 3000.

Rosetta Resolver Gene Expression Analysis system was used for microarray data analysis. Data quality control included Data Distribution Plot analysis; Hierarchical Clustering; and Data Reduction Analysis with Principal Components Analysis (PCA) applied to the data to produce a set of expression patterns known as principal components. No outliers were detected and all 12 samples were used in the data analyses.

Data Analysis

Statistical analysis (ANOVA) with multiple testing correction (FDR adjusted P*-value<0.001 and post hoc p-value<0.001) were used to generate a “stringent gene list” for the three post hoc comparisons (DP cells vs. DPfr, HDF, and HK, respectively) based on less stringent genes which passed filters of background correction and 3× standard deviations (>7.94) and ratio error p-value<0.01.

Candidate Validation

108 candidates from the stringent gene list were selected for further validation based on the relative levels of gene expression profiled below:

1. DPfr>DP cells>(HDF and HK)

2. DP cells>DPfr>(HDF and HK).

This further validation was performed by QPCR using standard methods. Validation was performed on both amplified and non-amplified RNA samples, and the results were very similar indicating that RNA amplification did not introduced significant variability in the samples.

A total of 80 candidates from the stringent gene list fit the desired gene expression profile in 1 or 2 above. Each candidate transcript is identified by one or more specific Affymetrix probe ID, which corresponds to a specific nucleotide sequence (target sequence).

Further Screening Using QPCR

Total RNA was prepared from hair induction-capable DP cells from 3 independent donors and from hair induction-incapable DP cells from 3 independent donors.

QPCR primers for the 80 candidate genes were designed based on the nucleotide sequence from which Affymetrix target nucleotide sequence was derived. QPCR analysis was performed by QPCR using standard methods.

Results

A number of sequences were identified that were expressed in hair inductive DP cells and/or DS cells and not in non-inductive DP cells and/or DS cells. Table 1 contains a list of these oligonucleotide marker sequences that are preferentially expressed in hair inductive DP/DS cells, together with associated sequence identifiers as used by various public databases.

TABLE 1
Oligonucleotide markers expressed in hair inductive DP cells and/or
DS cells but not in non-inductive DP cells and/or DS cells.

| Gene Name | Affymetrix | | RefSeq |
|---|---|---|---|
| SEQ ID NO | Probe ID | RefSeq ID | Protein ID |
| Serglycin (SRGN) | 201859_AT | NM_002727 | NP_002718 |
| SEQ ID NO: 1 | 201858_S_AT | | |
| Src-like-adaptor (SLA) | 203761_AT | NM_006748 | NP_001039021 |
| SEQ ID NO: 2 | | NM_001045556 | NP_001039022 |
| | | NM_001045557 | NP_006739 |
| Thrombomodulin (THBD) | 203887_S_AT | NM_000361 | NP_000352 |
| SEQ ID NO: 3 | 203888_AT | | |
| Runt-related | 236858_S_AT | NM_001015051 | NP_001015051 |
| transcription factor 2 (RUNX2) | 236859_AT | NM_001024630 | NP_001019801 |
| SEQ ID NO: 4 | | NM_004348 | NP_004339 |
| Runt-related | 204197_S_AT | NM_004350 | NP_001026850 |
| transcription factor 3 | 204198_S_AT | NM_001031680 | NP_004341 |
| (RUNX3) | | | |
| SEQ ID NO: 5 | | | |
| Protocadherin 17 | 205656_AT | NM_001040429 | NP_001035519 |
| SEQ ID NO: 6 | 228863_AT | | |
| Lymphocyte antigen | 205668_AT | NM_002349 | NP_002340 |
| 75 (LY75) | | | |
| SEQ ID NO: 7 | | | |
| Placental growth | 209652_S_AT | NM_002632 | NP_002623 |
| factor (PGF) | | | |
| SEQ ID NO: 8 | | | |
| Amyloid beta | 209870_S_AT | NM_005503 | NP_005494 |
| precursor protein- | | NM_001130414 | NP_001123886 |
| binding, family A, | | | |
| member 2 (APBA2) | | | |
| SEQ ID NO: 9 | | | |
| Prostaglandin E | 210367_S_AT | NM_004878 | NP_004869 |
| synthase (PTGES) | | | |
| SEQ ID NO: 10 | | | |
| myosin IF (MYO1F) | 213733_AT | NM_012335 | NP_036467 |
| SEQ ID NO: 11 | | | |
| G protein-coupled | 223767_AT | NM_020370 | NP_065103 |
| receptor 84 (GPR84) | | | |
| SEQ ID NO: 12 | | | |
| 230680_AT | 230680_AT | | |
| SEQ ID NO: 13 | | | |
| 232687_AT | 232687_AT | | |
| SEQ ID NO: 14 | | | |
| Transcription | 211276_AT | NM_080390 | NP_525129 |
| elongation factor A | | | |
| (SII)-like 2 (TCEAL2) | | | |
| SEQ ID NO: 15 | | | |
| Collagen, type XXIII, | 229168_AT | NM_173465 | NP_775736 |
| alpha 1 (COL23A1) | | | |
| SEQ ID NO: 16 | | | |
| ST8 alpha-N-acetyl- | 230261_AT | NM_005668 | NP_005659 |
| neuraminide alpha- | 242943_AT | NM_175052 | NP_778222 |
| 2,8-sialyltransferase 4 | | | |
| (ST8S1A4) | | | |
| SEQ ID NO: 17 | | | |
| 242303_AT | 242303_AT | | |
| SEQ ID NO: 18 | | | |
| Matrix | 207329_AT | NM_002424 | NP_002415 |
| metallopeptidase 8 | | | |
| (MMP8) | | | |
| SEQ ID NO: 19 | | | |
| Developmental | 219651_AT | NM_018189 | NP_060659 |
| pluripotency | 232985_S_AT | | |
| associated 4 (DPPA4) | | | |
| SEQ ID NO: 20 | | | |
| Endothelial cell- | 227780_S_AT | NM_001077693 | NP_001071161 |
| specific molecule 2 | | | |
| (ECSM2) | | | |
| SEQ ID NO: 21 | | | |

Certain oligonucleotide markers provided herein encode one or more polypeptides which can be used as polypeptide markers according to the invention. Specifically, these polypeptide markers are SEQ ID NO:s:22-46. Table 2 contains a list of these polypeptides marker sequences, together with associated oligonucleotide sequence identifiers as used by various public databases. As can be seen from Table 2, various oligonucleotide markers encode more than one polypeptide due to variations in mRNA splicing of the oligonucleotide marker.

TABLE 2
Polypeptide Markers encoded by oligonucleotide markers.

| Gene Name | |
|---|---|
| (SEQ ID NO) | RefSeq Protein ID (SEQ ID NO) |
| Serglycin (SRGN) | NP_002718 (SEQ ID NO: 22) |
| (SEQ ID NO: 1) | |
| Src-like-adaptor (SLA) | NP_001039021 (SEQ ID NO: |
| (SEQ ID NO: 2) | 23) |
| | NP_001039022 (SEQ ID NO: |
| | 24) |
| | NP_006739 (SEQ ID NO: 25) |
| Thrombomodulin (THBD) | NP_000352 (SEQ ID NO: 26) |
| (SEQ ID NO: 3) | |
| Runt-related transcription factor 2 | NP_001015051 (SEQ ID NO: |
| (RUNX2) | 27) |
| (SEQ ID NO: 4) | NP_001019801 (SEQ ID NO: |
| | 28) |
| | NP_004339 (SEQ ID NO: 29) |
| Runt-related transcription factor 3 | NP_001026850 (SEQ ID NO: |
| (RUNX3) | 30) |
| (SEQ ID NO: 5) | NP_004341 (SEQ ID NO: 31) |
| Protocadherin 17 (PCDH17) | NP_001035519 (SEQ ID NO: |
| (SEQ ID NO: 6) | 32) |
| Lymphocyte antigen 75 (LY75) | NP_002340 (SEQ ID NO: 33) |
| (SEQ ID NO: 7) | |
| Placental growth factor (PGF) | NP_002623 (SEQ ID NO: 34) |
| (SEQ ID NO: 8) | |
| Amyloid beta (A4) precursor protein- | NP_005494 (SEQ ID NO: 35) |
| binding, family A, member 2 (APBA2) | NP_001123886 (SEQ ID NO: |
| (SEQ ID NO: 9) | 36) |
| Prostaglandin E synthase (PTGES) | NP_004869 (SEQ ID NO: 37) |
| (SEQ ID NO: 10) | |
| myosin IF (MYO1F) | NP_036467 (SEQ ID NO: 38) |
| (SEQ ID NO: 11) | |
| G protein-coupled receptor 84 (GPR84) | NP_065103 (SEQ ID NO: 39) |
| (SEQ ID NO: 12) | |
| Transcription elongation factor A (SII)- | NP_525129 (SEQ ID NO: 40) |
| like 2 (TCEAL2) | |
| (SEQ ID NO: 15) | |
| Collagen, type XXIII, alpha 1 | NP_775736 (SEQ ID NO: 41) |
| (COL23A1) | |
| (SEQ ID NO: 16) | |
| ST8 alpha-N-acetyl-neuraminide alpha- | NP_005659 (SEQ ID NO: 42) |
| 2,8-sialyltransferase 4 (ST8SIA4) | NP_778222 (SEQ ID NO: 43) |
| (SEQ ID NO: 17) | |
| Matrix metallopeptidase 8 (MMP8) | NP_002415 (SEQ ID NO: 44) |
| (SEQ ID NO: 19) | |
| Developmental pluripotency associated | NP_060659 (SEQ ID NO: 45) |
| 4 (DPPA4) | |
| (SEQ ID NO: 20) | |
| Endothelial cell-specific molecule 2 | NP_001071161 (SEQ ID NO: |
| (ECSM2) | 46) |
| (SEQ ID NO: 21) | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for detecting trichogenic dermal cells comprising
contacting a population of skin cells with a binding moiety specific for Serglycin (SRGN), Src-like-adaptor—encoded polypeptide 3 (SLA), Thrombomodulin (THBD), Runt-related transcription factor 2 (RUNX2), Runt-related transcription factor 3 (RUNX3), Protocadherin 17 (PCDH17), Lymphocyte antigen 75 (LY75), Placental Growth Factor (PGF), Amyloid beta (A4) precursor protein-binding, family A, member 2 (APBA2), Prostaglandin E synthase (PTGES), myosin IF (MYO1F), G protein-coupled receptor 84 (GPR84), Transcription elongation factor A (SII)-like 2 (TCEAL2), Collagen, type XXIII, alpha 1 (COL23A1), ST8 alpha-N-acetyl-neuraminide alpha-2,8-sialyltransferase 4 (ST8SIA4), Matrix metallopeptidase 8 (MMP8), Developmental pluripotency associated 4 (DPPA4), Endothelial cell-specific molecule 2 (ECSM2), or a combination thereof; and
assaying for presence of the binding moiety on the skin cell, wherein detection of the binding moiety on the skin cell is indicative of the skin cell being a trichogenic dermal cell.

2. The method of claim 1, wherein the binding moiety is an antibody or antigen-binding fragment thereof.

3. The method of claim 1, wherein the binding moiety is labeled with a detectable label.

4. The method of claim 3, wherein the detectable label is selected from the group consisting of a radioisotope, fluorophore, or enzyme.

5. The method of claim 1, wherein in the binding moiety is detected using immunological detection, spectrophotometry, fluorospectroscopy, or mass spectroscopy.

6. The method of claim 1, wherein the trichogenic dermal cells are isolated using a cell sorting method.

7. The method of claim 6, wherein the cell sorting method is fluorescent-activated cell sorting (FACS) or magnetic bead cell sorting (MACS).

8. The method of claim 1, wherein the trichogenic dermal cells are isolated using a magnetic bead isolation.

9. A method for detecting trichogenic dermal cells in a skin cell population comprising
contacting a nucleic acid sample from a population of skin cells with an oligonucleotide primer or probe that hybridizes under stringent conditions to an oligonucleotide consisting of the nucleic acid sequence SEQ ID NO:1, 2, 3, 4, 5, 6, 7, 8, 9, 10, 11, 12, 13, 14, 15, 16, 17, 18, 19, 20, or 21, or the complement thereof; and
detecting hybridization of the oligonucleotide primer or probe to the nucleic acid sample, wherein detection of hybridization is indicative of trichogenic dermal cells being present in the skin cell population.

10. The method any one of claim 1, wherein the population of skin cells is derived from a culture of Dermal Papilla (DP) cells, Dermal Sheath (DS) cells, or a combination thereof.

11. A method for producing an enriched population of trichogenic dermal cells comprising
providing a heterogeneous skin cell population; and
selecting from the skin cell population cells expressing Serglycin (SRGN), Src-like-adaptor—encoded polypeptide 3 (SLA), Thrombomodulin (THBD), Runt-related transcription factor 2 (RUNX2), Runt-related transcription factor 3 (RUNX3), Protocadherin 17 (PCDH17), Lymphocyte antigen 75 (LY75), Placental Growth Factor (PGF), Amyloid beta (A4) precursor protein-binding, family A, member 2 (APBA2), Prostaglandin E synthase (PTGES), myosin IF (MYO1F), G protein-coupled receptor 84 (GPR84), Transcription elongation factor A (SII)-like 2 (TCEAL2), Collagen, type XXIII, alpha 1 (COL23AI), ST8 alpha-N-acetyl-neuraminide alpha-2,8-sialyltransferase 4 (ST8SIA4), Matrix metallopeptidase 8 (MMP8), Developmental pluripotency associated 4 (DPPA4), Endothelial cell-specific molecule 2 (ECSM2), or a combination thereof, thereby forming a second population of cells enriched for trichogenic dermal cells.

12. The method of claim 11, wherein the trichogenic dermal cells are dermal papilla cells, dermal sheath cells, or a combination thereof.

13. The method of claim 11, wherein the trichogenic dermal cells are selected using antibodies to SRGN, SLA, THBD, RUNX2, RUNX3, PCDH17, LY75, PGF, APBA2, PTGES, MYO1F, GPR84, TCEAL2, COL23A1, ST8SIA4, MMP8, DPPA4, or ECSM2 via fluorescence activated cell sorter or magnetic beads.

14. The method of claim 11, wherein the heterogeneous skin cell population is derived from the culture of dermal papilla cells, dermal sheath cells, or a combination thereof.

15. A population of trichogenic dermal cells produced by the method of claim 10.

16. A skin cell population comprising trichogenic dermal cells of claim 14 and epidermal cells.

17. The skin cell population of claim 16, wherein the epidermal cells are present in a ratio of epidermal to dermal cells effective to induce hair follicle formation when administered to a subject.

18. The skin cell population of claim 16 in a cell culture or shipping container.

19. The skin cell population of claim 16 in a device for injecting the cells into openings in the skin.

20. The skin cell population of claim 16, wherein the dermal cells are human dermal cells.

21. The skin cell population of claim 20, wherein the epidermal cells are human epidermal cells.

22. A method for inducing hair follicle formation in a subject comprising administering to the subject an effective amount of the cells of claim 14.

23. The method of claim 22, wherein the cells are administered to a site on the subject experiencing hair loss in an amount effective to induce hair follicle formation.

24. The method of claim 23 wherein the hair loss is due to androgenetic alopecia, wounding, trauma, scarring, telogen effluvium, genetic pattern baldness or with hormonal disorders that decrease hair growth or cause loss of hair.

25. The method of claim 22 wherein the cells administered to the subject are autologous or allogeneic cells.


SEQUENCE LISTING (46 sequences)

SEQ ID NO: 1 - DNA, 1270 bp, Homo sapiens
attttctaaaagggacagagagcaccctgctacatttcctaatcaagaagttggcgtgcagctgggagagctagactaagttggtcatgatgcagaagctactcaaatgcagtcggcttgtcctggctcttgccctcatcctggttctggaatcctcagttcaaggttatcctacgcggagagccaggtaccaatgggtgcgctgcaatccagacagtaattctgcaaactgccttgaagaaaaaggaccaatgttcgaactacttccaggtgaatccaacaagatcccccgtctgaggactgacctttttccaaagacgagaatccaggacttgaatcgtatcttcccactttctgaggactactctggatcaggcttcggctccggctccggctctggatcaggatctgggagtggcttcctaacggaaatggaacaggattaccaactagtagacgaaagtgatgctttccatgacaaccttaggtctcttgacaggaatctgccctcagacagccaggacttgggtcaacatggattagaagaggattttatgttataaaagaggattttcccaccttgacaccaggcaatgtagttagcatattttatgtaccatggttatatgattaatcttgggacaaagaattttatagaaatttttaaacatctgaaaaagaagcttaagttttatcatccttttttttctcatgaattcttaaaggattatgctttaatgctgttatctattttattgttcttgaaaatacctgcattttttggtatcatgttcaaccaacatcattatgaaattaattagattcccatggccataaaatggctttaaagaatatatatatatttttaaagtagcttgagaagcaaattggcaggtaatatttcatacctaaattaagactctgacttggattgtgaattataatgatatgccccttttcttataaaaacaaaaaaaaaaataatgaaacacagtgaatttgtagagtgggggtatttgacatattttacagggtggagtgtactatatactattacctttgaatgtgtttgcagagctagtggatgtgtttgtctacaagtatgattgctgttacataacaccccaaattaactcccaaattaaaacacagttgtgctgtcaatacctcatactgctttacctttttttcctggatatctgtgtattttcaaatgttactatatattaaagcagaaatataaccaaaggttaaaaaaaaaaaaaaaaaa

SEQ ID NO: 2 - DNA, 2872 bp, Homo sapiens
aaccaatcttcaccaatctcatcttcacatataaacagccgcctttcaagaagcaagctgccagaaaaatgatgcacgatgctctctaaactgggtcattctccacttggagggctcagggcacggttgactttccccgtctgtctcctataccacaggctctgggcatcaccagcggccccagggaaaaagaaagaaatgggaaacagcatgaaatccacccctgcgcctgccgagaggcccctgcccaacccggagggactggatagcgacttccttgccgtgctaagtgactacccgtctcctgacatcagccccccgatattccgccgaggggagaaactgcgtgtgatttctgatgaagggggctggtggaaagctatttctcttagcactggtcgagagagttacatccctggaatatgtgtggccagagtttaccatggctggctgtttgagggcctgggcagagacaaggccgaggagctgctgcagctgccagacacaaaggtcggctccttcatgatcagagagagtgagaccaagaaagggttttactcactgtcggtgagacacaggcaggtaaagcattaccgcattttccgtctgcccaacaactggtactacatttccccgaggctcaccttccagtgcctggaggacctggtgaaccactattctgaggtggctgatggcctgtgctgtgtgctcaccacgccctgcctgacacaaagcacggctgccccagcagtgagggcctccagctcacctgtcaccttgcgtcagaagactgtggactggaggagagtgtccagactgcaggaggaccccgagggaacagagaacccgcttggggtagacgagtcccttttcagctatggccttcgagagagcattgcctcttacctgtccctgaccagtgaggacaacacctcctttgatcgaaagaagaaaagcatctccctgatgtatggtggcagcaagagaaagagctcattcttctcatcaccaccttactttgaggactagccaagaacagacacaatggttcatgcccaaaaggaacagaagttccaactattgcctgggatcttgcgaaaagcgaggttccctgatccctgggagcctcacgtattttagaagccaagagaagccacatggagactcaaattcgcatcttctctatccacatcatgaccaaaggaacccctccctggtgtctgatcagggctgtggcatcacgaaacattggatcatgacatgtcgggcgatgcttggaagagcccagcatgtatgtatgcacacattgtgtgtgtgggaaggacaaagccactctcacaagaaagggcaccaggactgctctccaaggaactggacctgtccagacagttacactccaaggtcattggagagaacttctgtatgggcaagcctgagagggagaggaaacaaaagctgtgtcctggcagaaggtctgggtttgcagatgggtgccctgaatggaactactttaactaatccatagggacttctggtatgctttcctctctttttaaaggaacttcgtgacactaaacattagcccaaaggacttcttagccttcaattgggagatacctttggtctgctcctgcaccaaagccatatgggtggaagtcagttggcctccctggttctgcagagggccagaagaatgagagagaggaagactgctggcagggaaatcgaggaggcgagactagaactgcaccagcttccctgatgtctgcagccatggctttgcagcgcagacagagcttctctgggatgctgggattcttgcctgtatgaatgcatcaagtattcatttattgcccgaataggcattgcattaagtcctctgtaaggtgtcaggcaagccaaaaaaaaaaaaaagatgcgtaagtcctaacccccaacagaggtgttcacagtgtagacagggaaaaaatgtataaacaaatgtgtaaaaagagaaatcagctcatggcttaggatggaattagagacaggtgagggacactcaggagctcattttccagctgctcttcagagtggaagggctggctggatcgggtaggtaagaatagctggattttttagaaaagaaatggatacagtctaaagaattaactcacccggtactttattctaagaagggtctggcatccatatgaggaaaaatgctcagctccaggaaagatggggagtccaagtggattaatgatgtcatgcataattttaagagacaagggagaaaacacaatgtatagccagagaaggagaagctcccatccaaatcctactaggaagagagtgggctgcagatgaatctgtgactcatgtttccctgtttcaaagggatcctggggaaggaggggaacatgcttgcagtatctctccctgtctgtctgctcacataagcattccgtccatctgagctcatcgtgctactggtatgtgtatgtgcagttacacagttttctgtatcatagattctagtgtgtttatacaaggagacatctgtggtttccccaaccgttccaaaaggctatttcaaaggaaccagccaacgtatgagaaatgaatgtaacactgtggacattgacttcccgcataaggcagggtgaccccctgaactccagatgtctgcacagtatcttatgtgttgttttccgttgtgacgaatgtgattggaacatttggggagcacccagagggatttctcagtgggaagcattacactttgctaaatcatgtatttattcctgattaaaacaaacctaataaatatttaacccttggcaaaaaaaaaaaaaaaaaaaaaaaaaaaaaaaaaaaaaaaaaaaaaaaa

SEQ ID NO: 3 - DNA, 4048 bp, Homo sapiens
ggctgcctcgcaggggctgcgcgcagcggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagcctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcttagctgtctgctcagacagaacccctacatgaaacagaaacaaaaacactaaaaataaaaatggccatttgctttttcaccagatttgctaatttatcctgaaatttcagattcccagagcaaaataattttaaacaaaggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtgctctcgggttgtgtgtctgttcacttttcctccctcagtgccctcattttatgtcattaaatggggctcacaaaccatgcaaatgctatgagatgcatggagggctgccctgtaccccagcacttgtgttgtctggtggtggcaccatctctgattttcaaagctttttccagaggctattattttcactgtagaatgatttcatgctatctctgtgtgcacaaatatttattttctttctgtaaccataacaacttcatatatgaggacttgtgtctctgtgcttttaaatgcataaatgcattataggatcatttgttggaatgaattaaataaacccttcctggggcatctggcgaatcccaaaaaaaaaaaaaaaaa

SEQ ID NO: 4 - DNA, 5487 bp, Homo sapiens
gtgtgaatgcttcattcgcctcacaaacaaccacagaaccacaagtgcggtgcaaactttctccaggaggacagcaagaagtctctggtttttaaatggttaatctccgcaggtcactaccagccaccgagaccaacagagtcatttaaggctgcaagcagtatttacaacagagggtacaagttctatctgaaaaaaaaaggagggactatggcatcaaacagcctcttcagcacagtgacaccatgtcagcaaaacttcttttgggatccgagcaccagccggcgcttcagccccccctccagcagcctgcagcccggcaaaatgagcgacgtgagcccggtggtggctgcgcaacagcagcagcaacagcagcagcagcaacagcagcagcagcagcagcaacagcagcagcagcagcaggaggcggcggcggcggctgcggcggcggcggcggctgcggcggcggcagctgcagtgccccggttgcggccgccccacgacaaccgcaccatggtggagatcatcgccgaccacccggccgaactcgtccgcaccgacagccccaacttcctgtgctcggtgctgccctcgcactggcgctgcaacaagaccctgcccgtggccttcaaggtggtagccctcggagaggtaccagatgggactgtggttactgtcatggcgggtaacgatgaaaattattctgctgagctccggaatgcctctgctgttatgaaaaaccaagtagcaaggttcaacgatctgagatttgtgggccggagtggacgaggcaagagtttcaccttgaccataaccgtcttcacaaatcctccccaagtagctacctatcacagagcaattaaagttacagtagatggacctcgggaacccagaaggcacagacagaagcttgatgactctaaacctagtttgttctctgaccgcctcagtgatttagggcgcattcctcatcccagtatgagagtaggtgtcccgcctcagaacccacggccctccctgaactctgcaccaagtccttttaatccacaaggacagagtcagattacagaccccaggcaggcacagtcttccccgccgtggtcctatgaccagtcttacccctcctacctgagccagatgacgtccccgtccatccactctaccaccccgctgtcttccacacggggcactgggcttcctgccatcaccgatgtgcctaggcgcatttcaggtgcttcagaactgggccctttttcagaccccaggcagttcccaagcatttcatccctcactgagagccgcttctccaacccacgaatgcactatccagccacctttacttacaccccgccagtcacctcaggcatgtccctcggtatgtccgccaccactcactaccacacctacctgccaccaccctaccccggctcttcccaaagccagagtggacccttccagaccagcagcactccatatctctactatggcacttcgtcaggatcctatcagtttcccatggtgccggggggagaccggtctccttccagaatgcttccgccatgcaccaccacctcgaatggcagcacgctattaaatccaaatttgcctaaccagaatgatggtgttgacgctgatggaagccacagcagttccccaactgttttgaattctagtggcagaatggatgaatctgtttggcgaccatattgaaattcctcagcagtggcccagtggtatctgggggccacatcccacacgtatcaatatatacatatatagagagagtgcatatatatgtatatcgattagctatctacaaagtgcctattttttagaagatttttcattcactcactcagtcatgatcttgcagccataagagggtagatattgagaagcagaaggctcaagagagacaattgcaatcgagcttcagattgtttactatttaagatgtacttttacaaaggaacaaagaagggaaaaggtatttttgtttttgttgtttggtctgttatcatcaataacctgttcatatgccaattcagagaggtggactccaggttcaggagggagaagagcaaagccgcttcctctctgtgctttgaaacttcacaccctcacggtggcagctgtgtatggaccagtgccctccgcagacagctcacaaaaccagttgaggtgcactaaagggacatgaggtagaatggatgcttccatcacagtaccatcattcagaataactcttccaatttctgctttcagacatgctgcaggtcctcatctgaactgttgggttcgtttttttttttttttttcctgctccaagaaagtgacttcaaaaataactgatcaggatagattattttattttactttttaacactccttctccccttttcccactgaaccaaaaagaaatcccatccctaaaacctgccttctccttttatgcaaaactgaaaatggcaatacattattatagccataatggtatagatagtgattgcgtttggctatgtgttgttttctttttttttaaattatgaatatgtgtaaaatctgaggtaacttgctaacgtgaatggtcatataactttaaagatatatttataattatttaatgacatttggacccttgaaacatttcttagtgtattgatatgttgacttcggtctctaaaagtgctctttattaaataacaaatttcttcagtggtctagagccatatctgaaatattgctaagcaatttcagttcatccaggcacaatgtgattttaaaaaatacttccatctccaaatattttagatatagattgtttttgtgatgtatgaaggaaatgttatgtttagttctttcagatctttgaatgcctctaacacagctttgccttctaaagcggtaattagggatttaaaaaacaacctttagccctttatcagcatgaaatgctggagtgatgtggttttctaatttctttggggtaattatgactcttgtcatattaaaaagacaagcacaagtaaatcattgaactacagaaaaatgttctgtggtttcatagttaagcaaaactctaaatcgccaggcttcatagcaaagacatagtcagctaaaagccgcacatgtggatagagggttcaattatgagacacctagtacaggagagcaaaattgcaccagagattcttaaccaaccagccttaccaaacaacacaacaggggaaccccaatctgccttacccaaggccccactggcagctttccacagaatttgcatttagaggagcagaatgacatcactgtcctttgggagtaggtcctctgaaaaggcagcaggttccagcaggtagctgagctgagaggacatatggcccacggggacctacagacagcctttgacatttgtatttcttacaatggagggccaaggagggcaaggggctgtggagtttggtgtctactagtgtgtatgaatttgagctagagtccttctgtggcatgcactttgaccactcctggcagtcacatggcagatttccaagtgcaaatccttaatccaaacaaggatcatctaatgacaccaccaggccaatccctgctctcctccccgaaaagtcagggtcccttcattggaatcctccacccacccaagcagaatttagcagagatttgccttcaaaccctaacggcccccttgttctctggtccttctcaaacccacctttgtaggccacccagcattgcaggacagcgtgtggggcagctggacctgtgcttcctgcctgggagtctcccttggaattcatcctgactccttctaataaaaatggatgggaaagcaaaacactttgccttctaaaggccgtataccaagtatgcttagataaataagccacttttctattacttaagtaagaaggaagtagtaattgatactatttattgtttgtgtgtggtagcttgaagcacaccactgtccatttatttgtaagtgtaaaatatgtgtgtttgtttcagcagcacttaaaaaagccagtgtctggttacacatttcaattttaattaattgacataaaaatgctaccgccagtgccagctgcatcctatttaattaaaaaggtactatatttgtacattattttttaatgttaaaagggcttttttaagtttacagtacacataccgagtgactttagggatgcttttgtgttgaaatgttactatagtggctgcaggcagcaacccagaaacactttagaagctttttttccttgggaaaaattcaagcacttcttccctccaccctcactccaaccaccccaatgggggtaattcacatttcttagaacaaattctgcccttttttggtctagggattaaaattttgtttttctttctttctttttttttttttttcactgaacccttaatttgcactgggtcatgtgtttgatttgtgatttcaagaccaaagcaaagtcttactactactgtggaaccatgtactagttcctgggaattaaaatagcgtggttctctttgtagcacaaacattgctggaatttgcagtcttttcaatgcagccacatttttatccatttcagttgtctcacaaattttaacccatatcagagttccagaacaggtaccacagctttggttttagattagtggaataacattcagcccagaactgagaaactcaacagattaactatcgtttgctctttagacggtctcactgcctctcacttgccagagccctttcaaaatgagcagagaagtccacaccattagggaccatctgtgataaattcagaagggaggagatgtgtgtacagctttaaggattccctcaattccgaggaaagggactggcccagaatccaggttaatacatggaaacacgaagcattagcaaaagtaataattatacctatggtatttgaaagaacaataataaaagacacttcttccaaaccttgaatttgttgtttttagaaaacgaatgcatttaaaaatattttctatgtgagaattttttagatgtgtgtttacttcatgtttacaaataactgtttgctttttaatgcagtactttgaaatatatcagccaaaaccataacttacaataatttcttaggtattctgaataaaattccatttcttttggatatgctttaccattcttaggtttctgtggaacaaaaatatttgtagcattttgtgtaaatacaagctttcatttttattttttccaattgctattgcccaagaattgctttccatgcacatattgtaaaaattccgctttgtgccacaggtcatgattgtggatgagtttactcttaacttcaaagggactatttgtattgtatgttgcaactgtaaattgaattatttggcatttttctcatgattgtaatattaatttgaagtttgaatttaattttcaataaaatggcttttttggttttgtta

SEQ ID NO: 5 - DNA, 4270 bp, Homo sapiens
cccgccacttgattctggaggatttgttctggggctgcggccgcggagtcggggcggccgcgggcgagcttcggggcgggaggcggcggcagcggcacagccccgcgcgggccccgccgcggcccaggcagccgggacagccacgaggggcggccgcacgcggggccgcgcgccgaggatgcgggactagccgggcaggctgcgggcggccgtcgggccagcgaggcctcgcagcgggcgggccctggcgagtagtggccgggcgccgccccctgcgccctgaggcccgggccccgccgcttctgctttcccgcttctcgcggcagcggcggccgaggaggcgcccgcgccggccgcccccgggggaagccgcgccgtctccgcctgcccggcgccctgacggccgctgttatgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactgaccgccctggtggactcctcccgctggaggcggggaccctaacaaccttcaagaccagtgatgggccggctccgaggctccgggcgggaatgggacctgcgctccagggtggtctcggtcccagggtggtcccagctggtgggagcctctggctgcatctgtgcagccacatccttgtacagaggcataggttaccacccccaccccggcccgggatactgcccccggcccagatcctggccgtctcatcccatacttctgtggggaatcagcctcctgccacccccccggaaggacctcactgtctccagctatgcccagtgctgcatgggacccatgtctcctgggacagaggccatctctcttccagagagaggcagcattggcccacaggataagcctcaggccctgggaaacctcccgacccctgcaccttcgttggagcccctgcatcccctgggtccagccccctctgcatttacacagatttgagtcagaactggaaagtgtcccccacccccaccaccctcgagcggggttcccctcattgtacagatggggcaggacccagcacgctgctggcagagatggtttgagaacacatccaagccagtccccccagcccagcttcccctccgttcctaactgttggctttcccccagccgcacgggtcccaggccccagagaagatgagtctatggcatcaggttcttaaacccaggaaagcacctacagaccggctcctccatgcactttaccagctcaacgcatccactctctgttctcttggcagggcgggggaggggggataggaggtcccctttcccctaggtggtctcataattccatttgtggagagaacaggagggccagatagataggtcctagcagaaggcattgaggtgagggatcattttgggtcagacatcaatgtccctgtcccccctgggtccagccaagctgtgccccatcccccaagcctcctgggaggatccagccaaatcttgcgactcctggcacacacctgtctgtaacctgttttgtgctctgaaagcaaatagtcctgagcaaaaaaaaaaaaaaaacaaaaaaacaaaaaaaaaacaaaacagtttttaaaactgattttagaaaaagaagcttaatctaacgttttcaaacacaaggtctcttacaggtatagttccgtgattatgatagctctgtgattataagcaacatccccgccccctctcccccccgcggacccccagctgcctcctgagggtgtggggttattagggtctcaatactttctcaaggggctacactccccatcaggcagcatcccaccagcctgcaccacaggctcccctgggaggacgagggaaacgctgatgagacgctgggcatctctcctctgtggctctaggacatctgtccaggaggctgggcggaggtgggcaggatgtgagaggtggggagtactggctgtgcgtggcaggacagaagcactgtaaagggctctccagccgcagctcagctgcactgcgttccgaggtgaagtcttgcccctgaattttgcaaaatgggaaagtgggcgcttgcccaagggccaggctgcatggattctcacatcagagttctctggccctagaaaggcttagaaaaggcgtaagggaactcataaaggctagcagcatgcggtattttaactttctgcctcggcctctgtggatgcagaaatctgccctacaaaatgctcttcattggttgtctctgtgagagcactgtccccacccaacctgtcacaacggccagaaccatacaccagagacacactggcaggttaggcagtccttctggtgatcctattccattccctcctgctgcggtttctcttggcctgtcctcactggaaaaacagtctccatctcctcaaaatagttgctgactccctgcacccaaggggcctctccatgccttcttaggaagcagctatgaatccattgtccttgtagtttcttccctcctgttctctggttatagctggtcccaggtcagcgtgggaggcacctttgggttcccagtgcccagcactttgtagtctcatcccagattactaacccttcctgatcctggagaggcagggatagtaaataaattgctcttcctaccccatcccccatcccctgacaaaaagtgacggcagccgtactgagtctgtaaggcccaaagtgggtacagacagcctgggctggtaaaagtaggtccttatttacaaggctgcgttaaagttgtactaggcaaacacactgatgtaggaagcacgaggaaaggaagacgttttgatatagtgttactgtgagcctgtcagtagtgggtaccaatcttttgtgacatattgtcatgctgaggtgtgacacctgctgcactcatctgatgtaaaaccatcccagagctggcgagaggatggagctgggtggaaactgctttgcactatcgtttgcttggtgtttgtttttaacgcacaacttgcttgtacagtaaactgtcttctgtactatttaactgtaaaatggaattttgactgatttgttacaataatataactctgagatgtgtggaagga

SEQ ID NO: 6 - DNA, 8009 bp, Homo sapiens
gatttcgggggagagccttttccgaggaagagagggaggagcctggtggggagaggaaactacaaatcgggacactagttctttacgctgcatttcctcccctccctttggctgctcggaaaggagagagaggaaaaaaaaaatacgcttggctggtagatgcagtccgccgccgccgctgcctcagccagcaatgcaagattagatctctaaatgcagcaaaacactgcctgaaaacagaccggcccgcgcagcaagcagacatttcacggtgcgctggggaagcttcaaaatatatctgtgactctgtcttcgttgctcttcatccccatcaatttcatcacgggaggcgagcagcaagtaagaatttcactttcggatctgcctagagacacacctccctgctccctcccccactcgatgtgaagagtattccggagtctccgggcgggagtagatttgcagcaccctagcgggagcgaggaaaacctactgattctttagctcattatcatctctcccagacgagatttccttcttatcgcctgcctcatcgctcaagtttgagcctcccgaagtccgggcgggagagacgaaacccctggctcacccccagccgcaggaagccaccgccttgctccaagcccctgcagctctgctgcaccgcagcttctcacccagtgcggatgctgtagatcaacaggttcagggaacttgagcagaataaggagagaccaccgggtgccgcagctcgggtgcagagggaaaaaaggacccatagacttgtggctcgcgtcgcgcgcgcacgctgcgccagggccccaggctggcgcgcactccctctctggctcctccagtccgattgctcctgcccccaccttacaggtctgggatgtacctttccatctgttgctgctttcttctatgggcccctgccctcactctcaagaacctcaactactccgtgccggaggagcaaggggccggcacggtgatcgggaacatcggcagggatgctcgactgcagcctgggcttccgcctgcagagcgcggcggcggagggcgcagcaagtcgggtagctaccgggtgctggagaactccgcaccgcacctgctggacgtggacgcagacagcgggctcctctacaccaagcagcgcatcgaccgcgagtccctgtgccgccacaatgccaagtgccagctgtccctcgaggtgttcgccaacgacaaggagatctgcatgatcaaggtagagatccaggacatcaacgacaacgcgccctccttctcctcggaccagatcgaaatggacatctcggagaacgctgctccgggcacccgcttccccctcaccagcgcacatgaccccgacgccggcgagaatgggctccgcacctacctgctcacgcgcgacgatcacggcctctttggactggacgttaagtcccgcggcgacggcaccaagttcccagaactggtcatccagaaggctctggaccgcgagcaacagaatcaccatacgctcgtgctgactgccctggacggtggcgagcctccacgttccgccaccgtacagatcaacgtgaaggtgattgactccaacgacaacagcccggtcttcgaggcgccatcctacttggtggaactgcccgagaacgctccgctgggtacagtggtcatcgatctgaacgccaccgacgccgatgaaggtcccaatggtgaagtgctctactctttcagcagctacgtgcctgaccgcgtgcgggagctcttctccatcgaccccaagaccggcctaatccgtgtgaagggcaatctggactatgaggaaaacgggatgctggagattgacgtgcaggcccgagacctggggcctaaccctatcccagcccactgcaaagtcacggtcaagctcatcgaccgcaacgacaatgcgccgtccatcggtttcgtctccgtgcgccagggggcgctgagcgaggccgcccctcccggcaccgtcatcgccctggtgcgggtcactgaccgggactctggcaagaacggacagctgcagtgtcgggtcctaggcggaggagggacgggcggcggcgggggcctgggcgggcccgggggttccgtccccttcaagcttgaggagaactacgacaacttctacacggtggtgactgaccgcccgctggaccgcgagacacaagacgagtacaacgtgaccatcgtggcgcgggacgggggctctcctcccctcaactccaccaagtcgttcgcgatcaagattctagacgagaacgacaacccgcctcggttcaccaaagggctctacgtgcttcaggtgcacgagaacaacatcccgggagagtacctgggctctgtgctcgcccaggatcccgacctgggccagaacggcaccgtatcctactctatcctgccctcgcacatcggcgacgtgtctatctacacctatgtgtctgtgaatcccacgaacggggccatctacgccctgcgctcctttaacttcgagcagaccaaggcttttgagttcaaggtgcttgctaaggactcgggggcgcccgcgcacttggagagcaacgccacggtgagggtgacagtgctagacgtgaatgacaacgcgccagtgatcgtgctccccacgctgcagaacgacaccgcggagctgcaggtgccgcgcaacgctggcctgggctatctggtgagcactgtgcgcgccctagacagcgacttcggcgagagcgggcgtctcacctacgagatcgtggacggcaacgacgaccacctgtttgagatcgacccgtccagcggcgagatccgcacgctgcaccctttctgggaggacgtgacgcccgtggtggagctggtggtgaaggtgaccgaccacggcaagcctaccctgtccgcagtggccaagctcatcatccgctcggtgagcggatcccttcccgagggggtaccacgggtgaatggcgagcagcaccactgggacatgtcgctgccgctcatcgtgactctgagcactatctccatcatcctcctagcggccatgatcaccatcgccgtcaagtgcaagcgcgagaacaaggagatccgcacttacaactgccgcatcgccgagtacagccacccgcagctgggtgggggcaagggcaagaagaagaagatcaacaaaaatgatatcatgctggtgcagagcgaagtggaggagaggaacgccatgaacgtcatgaacgtggtgagcagcccctccctggccacctcccccatgtacttcgactaccagacccgcctgcccctcagctcgccccggtcggaggtgatgtatctcaaaccggcctccaacaacctgactgtccctcaggggcacgcgggctgccacaccagcttcaccggacaagggactaatgcaagcgagacccctgccactcggatgtccataattcagacagacaattttcccgcagagcccaattacatgggcagcaggcagcagtttgttcaaagtagctccacgtttaaggacccagaaagagccagcctgagagacagtgggcacggggacagtgatcaggctgacagtgaccaagacactaacaaaggctcctgctgtgacatgtctgttagggaggcactcaagatgaaaactacttcaactaaaagccaaccacttgaacaagaaccagaagagtgtgttaattgcacagatgaatgccgagtgcttggtcattctgacaggtgctggatgccacagttccctgcagccaatcaggctgaaaatgcagattaccgcacaaatctctttgtacctacagttgaagctaatgttgagactgagacttacgaaactgtgaatcccactgggaaaaagactttttgtacatttggaaaagacaagcgagagcacactattctcattgccaacgttaaaccttatttaaaagccaaacgtgccctgagccctctcctccaagaggtcccctcagcatcaagcagcccaaccaaggcgtgcatcgagccttgcacctcaacaaaaggctccctggatggctgtgaagcaaaaccaggagccctggctgaagcaagcagtcagtacttgcccactgacagtcaatatctgtcacctagtaagcaaccaagagaccctcccttcatggcttccgatcagatggcaagggtctttgcagatgtgcattccagagccagccgggattccagtgagatgggtgctgttcttgagcagcttgaccaccccaacagggatctgggcagagagtctgtggatgcagaggaagttgtgagagaaattgataagcttttgcaagactgccggggaaacgaccctgtggctgtgagaaagtgaaaaaagaaaaaaaaaaaggcattggcattttcttgtctcttctgttgatttaaaaatgatccctcctggtgataacccattttacagggatgaagaaagaccaatgctgctttaaggcttttagtgaacatctgaagtgcccacaagtatgttctttccactgctgatttctttttcagagataacaatggtttcgttttgaccaaacttgtattaggacagaattaatgatgcttaaagagaaaagaaaaaaagagagaagaaaaaggagagatgaaaaaggaggatgaggagaagaattaccttttgacaatctgttaggaaggtatgcagtgtgagaactgaagtatttctgatcactctcagactgtcctccgtgatttatgctgacttaactgtttacctataaaccccatacaaagcagggtcataatttgtgatctgtggtggatttctagcagtcatcacaggcttctactgaaagtcctgaaaagaccttgcagtagtccaagctacaccaaacattaacacatatttgtggtaaacatttctgtataaagttacctgacacacatataaacacaaggaacattccatatcattagtcgaaaacaaaaacaaaaaaaaaacctttggtcatttgtaagacatctcatgtcatataaaagttaaatgtaaaaagatacagtccattttgtcctgcacacacgtagactaattcacgtcattaaagaagaagaaaacttaaagatttaaaatgcctatttagcattttagtgtccaacaaagatttaaacaatgatgaatatgttttaaatttgacatagaaaagttctaaaaaatagttaccattgagtggtaagattcagagaaaattaacttgattaatatgttttattcatttgtggacactaaaatagctcaggaaagtgaaaatgtcttagacatacgcaagtcacatgaccatttaaatgtgcaaatgtaagaagattcaatgtgtttacatcaaatgacatattttattgatttattgcagattcagtgcatatgagccaaattgttgagtgtgtaagagctatattgtgtattttattaaattaatatatagttgtgttgcaaaaatatttgggcttatattgtaaatggcaagtgttgccttggtagctgtcgaactctatgagttttgttttttcctgcttccttttccccatggagtgtgggaagcagtgcctcagagcaaagtctcttgtttaatgtatagtctaccaagtactacagtacataatctgttcaaaatgtgtttgagtgagctgatggagctaactgaaaggtcaaaaattacatccatcagtcatggttatgtgcaagtccttgtagaagcttttattaaagtcatgctaaatcacaagaattgacatttgtaccaatatctgaaacttcttcatgttttttcaataacatacagcttctgcctgtgtagatattatgccatcagttggttctcaaaagtattttaagtgcttcagatgtgtgttcccattatattttgaaaacatgaaaaatgctttaatgcatgtatgtaccagcagtggttacttgcattgtgtagtgtttttcaagaggtctgggtcttaacaaaatgttttcctttatctcagtgctcttctgcctctttttgttggtgtcctttgagaacaatacaccttctattccttcatttggttacacctttccttgtgacatttagcgagtttcaaacttacttccatatgaggctaagaaacctcaaatttcaggaattgggaaaaataaaattagcacttgcagaagtagcagcagatgggaaaatgccttgattgacattttctttcagcatttaaaatttttggcattttacagcttcatgacaaacagttttgtgcccataccttagaaaatgtggtgctgagttaaataaaggctgtttgagcactggagcagaaaaatgcattatttgcaaactggtggataattttgtgccttctcttctggccaccaagccagtgtagaaacagcaaaaatgtcataaaaattcttatatttaaaacaaaaacaaaagcaaaaacaaacattgaattaaattaagttttgtaattttaaactttaaaaacttctactgaaaatatttccgccaaatgccatcaatattttagactgtacctcgtttgcaaaactgctttgagagggaagagtggacaactcccatcagccttattctcttgagaactatattttggttcctagtaacagcctttccaaagctctactcttggtttttattactcataaatgtttaaattagaaaagaagggaccttgtacatgtgaaacctaattgactctctatattttggacaatttatgtatctgaaatgtgttgtctctgttatatgatgttatttttgccaggagactacaggttgatttagcttgatagctgaaatttgatggaaaactgatttccatttagtcttaccaagtgttgcttctctcttactagacagatatccacttagtaaaatctaaagcagtatgtaaatgaaaccagcaaagagagtagggtttattttataaacattcttaatgctaagtaaccagttgttcaatttattatatgtgtctgaggacattaaaacaccataaggttgtaataattggttgtgccaatgtgtgagggatttacctttaggctctctgtcaccagtgatttactagtgttagctgtttaacacattatctgtatttagtagtgattatttatttacaagttggtggtaattcagcagtcaggactctaagcttttatagttgaattgaggaaatctcgcttttattcatttagctggcaactgcctttattgcagacctctggtgcttggctttcaaggaagcctatgagatgccaaaatcacacctttagagagcaccttgctctaataggtgatgcatgagcaaacagtgagatttgaaggggttttaacataatttagaatgtgaaaaaaatatcaattcatatctttcaagtactaacccctcaaaaaagcccacacatacaaaatatgtgatgtgataccactttgtcttttaggtctttaagtaactgaagttaagcacagaaaaaaaaatcacttcatggaaatttcagtaagaaacccaaacttctaaaaattgcttgcagatgagctaaaaaaaaaaaaaaaaaaaaaaagcaacaaaataaccttttcatcagagttaaaagtagtgagaatctgttagttatacgtataccaaagtaaacattaaagagacatatcatgcaatttcaaagaattctttcatgctatttcttaacctgacatttctaactttattgcaggcaatatacaaagattggctcactactccataggttaattgaattcctggttgagaaactaacttgttttgttttccaaaattagctgaaatcttgtaaaacatgacttccctttaaaggatctagatattgttcaatttaaaatatggcaccataaaaaagtcatgtagtaatagagcatatgcttttttagaaccaggttaaaagctgtttgttatctaatagagtaaaagttactgag

SEQ ID NO: 7 - DNA, 6927 bp, Homo sapiens
gcgctcagcaggcggggcgggagccgcgtgcgcccgaggacccggccggaaggcttgcgccagctcaggatgaggacaggctgggcgacccctcgccgcccggcggggctcctcatgctgctcttctggttcttcgatctcgcggagccctctggccgcgcagctaatgaccccttcaccatcgtccatggaaatacgggcaagtgcatcaagccagtgtatggctggatagtagcagacgactgtgatgaaactgaggacaagttatggaagtgggtgtcccagcatcggctctttcatttgcactcccaaaagtgccttggcctcgatattaccaaatcggtaaatgagctgagaatgttcagctgtgactccagtgccatgctgtggtggaaatgtgagcaccactctctgtacggagctgcccggtaccggctggctctgaaggatggacatggcacagcaatctcaaatgcatctgatgtctggaagaaaggaggctcagaggaaagcctttgtgaccagccttatcatgagatctataccagagatgggaactcttatgggagaccttgtgaatttccattcttaattgatgggacctggcatcatgattgcattcttgatgaagatcatagtgggccatggtgtgccaccaccttaaattatgaatatgaccgaaagtggggcatctgcttaaagcctgaaaacggttgtgaagataattgggaaaagaacgagcagtttggaagttgctaccaatttaatactcagacggctctttcttggaaagaagcttatgtttcatgtcagaatcaaggagctgatttactgagcatcaacagtgctgctgaattaacttaccttaaagaaaaagaaggcattgctaagattttctggattggtttaaatcagctatactctgctagaggctgggaatggtcagaccacaaaccattaaactttctcaactgggatccagacaggcccagtgcacctactataggtggctccagctgtgcaagaatggatgctgagtctggtctgtggcagagcttttcctgtgaagctcaactgccctatgtctgcaggaaaccattaaataatacagtggagttaacagatgtctggacatactcagatacccgctgtgatgcaggctggctgccaaataatggattttgctatctgctggtaaatgaaagtaattcctgggataaggcacatgcgaaatgcaaagccttcagtagtgacctaatcagcattcattctctagcagatgtggaggtggttgtcacaaaactccataatgaggatatcaaagaagaagtgtggataggccttaagaacataaacataccaactttatttcagtggtcagatggtactgaagttactctaacatattgggatgagaatgagccaaatgttccctacaataagacgcccaactgtgtttcctacttaggagagctaggtcagtggaaagtccaatcatgtgaggagaaactaaaatatgtatgcaagagaaagggagaaaaactgaatgacgcaagttctgataagatgtgtcctccagatgagggctggaagagacatggagaaacctgttacaagatttatgaggatgaggtcccttttggaacaaactgcaatctgactatcactagcagatttgagcaagaatacctaaatgatttgatgaaaaagtatgataaatctctaagaaaatacttctggactggcctgagagatgtagattcttgtggagagtataactgggcaactgttggtggaagaaggcgggctgtaaccttttccaactggaattttcttgagccagcttccccgggcggctgcgtggctatgtctactggaaagtctgttggaaagtgggaggtgaaggactgcagaagcttcaaagcactttcaatttgcaagaaaatgagtggaccccttgggcctgaagaagcatcccctaagcctgatgacccctgtcctgaaggctggcagagtttccccgcaagtctttcttgttataaggtattccatgcagaaagaattgtaagaaagaggaactgggaagaagctgaacgattctgccaagcccttggagcacacctttctagcttcagccatgtggatgaaataaaggaatttcttcactttttaacggaccagttcagtggccagcattggctgtggattggtttgaataaaaggagcccagatttacaaggatcctggcaatggagtgatcgtacaccagtgtctactattatcatgccaaatgagtttcagcaggattatgacatcagagactgtgctgctgtcaaggtatttcataggccatggcgaagaggctggcatttctatgatgatagagaatttatttatttgaggccttttgcttgtgatacaaaacttgaatgggtgtgccaaattccaaaaggccgtactccaaaaacaccagactggtacaatccagaccgtgctggaattcatggacctccacttataattgaaggaagtgaatattggtttgttgctgatcttcacctaaactatgaagaagccgtcctgtactgtgccagcaatcacagctttcttgcaactataacatcttttgtgggactaaaagccatcaaaaacaaaatagcaaatatatctggtgatggacagaagtggtggataagaattagcgagtggccaatagatgatcattttacatactcacgatatccatggcaccgctttcctgtgacatttggagaggaatgcttgtacatgtctgccaagacttggcttatcgacttaggtaaaccaacagactgtagtaccaagttgcccttcatctgtgaaaaatataatgtttcttcgttagagaaatacagcccagattctgcagctaaagtgcaatgttctgagcaatggattccttttcagaataagtgttttctaaagatcaaacccgtgtctctcacattttctcaagcaagcgatacctgtcactcctatggtggcacccttccttcagtgttgagccagattgaacaagactttattacatccttgcttccggatatggaagctactttatggattggtttgcgctggactgcctatgaaaagataaacaaatggacagataacagagagctgacgtacagtaactttcacccattattggttagtgggaggctgagaataccagaaaatttttttgaggaagagtctcgctaccactgtgccctaatactcaacctccaaaaatcaccgtttactgggacgtggaattttacatcctgcagtgaacgccactttgtgtctctctgtcagaaatattcagaagttaaaagcagacagacgttgcagaatgcttcagaaactgtaaagtatctaaataatctgtacaaaataatcccaaagactctgacttggcacagtgctaaaagggagtgtctgaaaagtaacatgcagctggtgagcatcacggacccttaccagcaggcattcctcagtgtgcaggcgctccttcacaactcttccttatggatcggactcttcagtcaagatgatgaactcaactttggttggtcagatgggaaacgtcttcattttagtcgctgggctgaaactaatgggcaactcgaagactgtgtagtattagacactgatggattctggaaaacagttgattgcaatgacaatcaaccaggtgctatttgctactattcaggaaatgagactgaaaaagaggtcaaaccagttgacagtgttaaatgtccatctcctgttctaaatactccgtggataccatttcagaactgttgctacaatttcataataacaaagaataggcatatggcaacaacacaggatgaagttcatactaaatgccagaaactgaatccaaaatcacatattctgagtattcgagatgaaaaggagaataactttgttcttgagcaactgctgtacttcaattatatggcttcatgggtcatgttaggaataacttatagaaataagtctcttatgtggtttgataagaccccactgtcatatacacattggagagcaggaagaccaactataaaaaatgagaagtttttggctggtttaagtactgacggcttctgggatattcaaacctttaaagttattgaagaagcagtttattttcaccagcacagcattcttgcttgtaaaattgaaatggttgactacaaagaagaatataatactacactgccacagtttatgccatatgaagatggtatttacagtgttattcaaaaaaaggtaacatggtatgaagcattaaacatgtgttctcaaagtggaggtcacttggcaagcgttcacaaccaaaatggccagctctttctggaagatattgtaaaacgtgatggatttccactatgggttgggctctcaagtcatgatggaagtgaatcaagttttgaatggtctgatggtagtacatttgactatatcccatggaaaggccaaacatctcctggaaattgtgttctcttggatccaaaaggaacttggaaacatgaaaaatgcaactctgttaaggatggtgctatttgttataaacctacaaaatctaaaaagctgtcccgtcttacatattcatcaagatgtccagcagcaaaagagaatgggtcacggtggatccagtacaagggtcactgttacaagtctgatcaggcattgcacagtttttcagaggccaaaaaattgtgttcaaaacatgatcactctgcaactatcgtttccataaaagatgaagatgagaataaatttgtgagcagactgatgagggaaaataataacattaccatgagagtttggcttggattatctcaacattctgttgaccagtcttggagttggttagatggatcagaagtgacatttgtcaaatgggaaaataaaagtaagagtggtgttggaagatgtagcatgttgatagcttcaaatgaaacttggaaaaaagttgaatgtgaacatggttttggaagagttgtctgcaaagtgcctctgggccctgattacacagcaatagctatcatagttgccacactaagtatcttagttctcatgggcggactgatttggttcctcttccaaaggcaccgtttgcacctggcgggtttctcatcagttcgatatgcacaaggagtgaatgaagatgagattatgcttccttctttccatgactaaattcttctaaaagttttctaatttgcactaatgtgttatgagaaattagtcacttaaaatgtcccagtgtcagtatttactctgctccaaagtagaactcttaaatactttttcagttgtttagatcttaggcatgtgctggtatccacagttaattccctgctaaatgccatgtttatcaccctaattaatagaatggaggggactccaaagctggaactgaagtccaaattgtttgtacagtaatatgtttaatgttcattttctctgtatgaatgtgattggtaactaggatatgtatattttaatagaatttttaacaaaacttcttagaaaattaaaataggcatattactaggtgacatgtctactttttaatttttaagagcatccggccaaatgcaaaattagtacctcaaagtaaaaattgaactgtaaactctatcagcattgtttcaaaatagtcatttttagcactggggaaaaataaacaataagacatgcttactttttaatttttatttttttgagactgagtctctctctgttgcccaggctggagtacaatggcgtgatctcggctcactgcaaatctccgcctcccaggttcaagcgattctcctgcctcagcctcctgagtagctgggattacaggcaactgccaccatgcccggctaatttttgtatttttagtagagatggggtttcaccatgttggccaggctggtctcgaactcgtgaccgcaggtgatcctcccgcctcggcctcccaaagtgctgggattacaggcatgagccaccgcgcctggcctctgcttactttttatatagcaaaatgattcctcttggcaagatgtttcttatattattccaaagttatttcataccattattatgtaaatatgaagagtttttttctgtttataattgtttataaaacaatgacttttaaagatttagtgcttaacattttcccaagtgtgggaacattatttttagattgagtaggtaccttgtagcagtgtgctttgcattttctgatgtattacatgactgtttcttttgtaaagagaatcaactaggtatttaagactgataattttacaatttatatgcttcacatagcatgtcaacttttgactaagaattttgttttacttttttaacatgtgttaaacagagaaagggtccatgaaggaaagtgtatgagttgcatttgtaaaaatgagactttttcagtggaactctaaaccttgtgatgactactaacaaatgtaaaattatgagtgattaagaaaacattgctttgtggttatcactttaagttttgacacctagattatagtcttagtaatagcatccactggaaaaggtgaaaatgttttattcggcatttaacttacatttgtactttatttttgtataaaatccatagatttattttacatttagagtatttacactatgataaagttgtaaataattttctaagacagtttttatatagtctacagttgtcctgatttcttattgaatttgttagactagttctcttgtcctgtgatctgtgtacaattttagtcactaagactttcctccaagaactaagccaacttgatgtgaaaagcacagctgtatataatggtgatgtcataataaagttgttttatcttttaagtaaaagtaaaa

SEQ ID NO: 8 - DNA, 1758 bp, Homo sapiens
ctgctgtctgcggaggaaactgcatcgacggacggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggaggagacccaagggcagggggaagaggaggagagagaagcagagacccacagactgccacctgtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggatcctgctggggccgccacggcctggctggtgggaaggccggcagcgggcggaggggatccagccacttccccctcttcttctgaagatcagaacattcagctctggagaacagtggttgcctgggggcttttgccactccttgtcccccgtgatctcccctcacactttgccatttgcttgtactgggacattgttctttccggccaaggtgccaccaccctgccccccctaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaaaaaaaaaaaaaaaaaaaaaaaaaaaaa

SEQ ID NO: 9 - DNA, 3653 bp, Homo sapiens
cagtctcctgaatatttacgcgttgctgaatctcctgtggacaaaccaccaataggccaggactgtcctgtggacagacggggtgagcctcttcttgtgtctggagattctgaaaagatttgatcaccaggagatttttcgggacattaccaaaccactcattttagtggctgcctccgggtgatgatggctgtgtgaacgactgccatggcccaccggaagcttgagagcgtggggagcggcatgttggaccatagggtgagaccaggtcctgtccctcacagccaggagcccgagagcgaggacatggagctgcccttggagggctatgtgcccgagggcctggagctggctgccctgcggccagagagccccgcgccagaggaacaggagtgccacaaccacagccccgatggggactccagctctgactacgtgaacaacacctctgaggaggaggactatgacgagggcctccctgaggaggaggagggcatcacctactacatccgctactgccctgaggacgacagctacctagagggcatggactgcaacggggaggagtacctggcccacagtgcacaccctgtggacactgatgagtgccaggaggcggtggaggagtggacggactcggcgggcccgcacccccacggccacgaggctgaaggcagccaggactacccagacggccaactgcccattccggaggatgagccctccgtccttgaggcccatgaccaggaagaagatggtcactactgtgccagcaaagagggctaccaggactactaccccgaggaggccaacgggaacaccggcgcctccccctaccgcctgaggcgtggggatggggacctggaggaccaggaggaggacattgaccagatcgtggcagagatcaagatgagtctgagcatgaccagcatcaccagcgccagtgaggccagccccgagcatgggcctgagccagggcctgaggactctgtagaggcctgcccacccatcaaggccagctgcagccccagcaggcacgaggcgaggcccaagtcgctgaacctccttcccgaggccaagcaccccggagacccccagagaggcttcaagcccaagaccaggaccccagaagagaggctgaagtggccccacgagcaggtttgcaatggtctggagcagccaaggaagcagcagcgctctgatctcaatggacctgttgacaataacaacattccagagacaaagaaggtggcatcatttccaagttttgtggctgttccagggccctgcgaaccagaagacctcatcgacgggatcatctttgctgccaattacctggggtccacccagctgctatcagaacggaacccttccaaaaacatcagaatgatgcaagcgcaggaggccgtcagccgggtcaagaggatgcaaaaggctgctaagatcaagaaaaaagcgaattctgagggggatgcccagacgctgacggaagtggacctcttcatttccacccagaggatcaaggttttaaatgcagacacgcaggaaaccatgatggaccacgccttgcgtaccatctcctacatcgccgacattgggaacattgtagtgctgatggccagacgccgcatgccccggtcagcctctcaggactgcatcgagaccacgcccggggcccaggaaggcaagaagcagtataagatgatctgccatgtgttcgagtcggaggatgcccagctcatcgcccagtctatcggccaggccttcagcgtggcctaccaggagttcctgcgagccaatggcatcaaccccgaagacttgagccagaaggaatacagcgacatcatcaacacccaggagatgtacaacgacgacctcatccacttctcaaactcggagaactgcaaggagctgcagctggagaagcacaagggcgagatcctgggcgtggtggtggtggagtcgggctggggctccatcctgcccacggtgatcctggccaacatgatgaatggcggcccggctgcccgctcggggaagctgagcatcggggaccagatcatgtccatcaatggcaccagcctggtggggctgcccctcgccacctgccaaggcatcatcaagggcctgaagaaccagacacaggtgaagctcaacattgtcagctgtcccccggtcaccacggtccttatcaagcggccagacctcaagtaccagctgggcttcagcgtgcagaatggaattatctgcagcctcatgagagggggcattgctgagcgagggggcgtccgtgtgggccaccgcatcatcgagatcaacgggcagagcgtggtggccacagcccacgagaagatagtccaagctctgtccaactcggtcggagagatccacatgaagaccatgcccgccgccatgttcaggctcctcacgggtcaggagaccccgctgtacatctaggccaccccagcctggccacgcagccaggacaccgggcagggccgcccgggcccagaggagctgggagccgggccgcagacttgaccccgacgccacagcccagccacggacgctggctccccaaagggtgtgccctcaccacccacttgatttttttcattttgccaaaaaggggtatgtctttatcaaaggagagtcacagaacaaatgtttgtttgtaaagcgttccaagtattttgccacgttctggactgtcttctccctgcacaagccagggtgtgtctcggtagctgtgcgtggtgtggagtgtgtgtctttcctccctgaagctgtgcggagcgaactggcgcctccgagggacgcggctcccggggcagggcagccgtcacccctgcctcccgcccccttggctgggacgtctggggtcctgtggggcccccacaatggtcccaaacagctgcctctgccactgactgcagggacacgggcagcctggctcccaggacacgacttgtaatgaaagtttggggacatgtgattgattgattgattgtaaataaaggatgatggccacaacatgaaaactccatatttatttagatgctattattactgtttggacttttattttggcaggcttttttccagactctagggttttccaatgtgactaatgaccacacctgcctctcccgtcgtctcttctgggcaccctcccacccggctgcatacccgggcagggctcccacagagacaaggagggcacaggtgtctgccccctctttaaaatcgatctacacacatccacgcacatgcgaccccgaggaaacgaaacccactctagaaaacgcgaccttggccgcacctaaagcagccagccgtgagtgcagaccccttggccagcgtggcgcagtggccctgagcagtagtggcatgtgtgtagatcaagtcggatctagtccagctcggttcattagcgatccatgtaatctgacgtcatcttgtctcgaagtctctttttttggcccaggccttgaagaatacactgtgacttaagaagccttaccacgcagtaactaaagctttaggatgactgtattcgaggagtgccgtgtgttgcatgcagctacccgtaggaagacttcgcgcatatcactaataaacctgaagtcgtgatgaaaaaaaaaaaaaaaaaa

SEQ ID NO: 10 - DNA, 1787 bp, Homo sapiens
gctgctcctctgtcgagctgatcacacccacagttgagctgcgctggccagagatgcctgcccacagcctggtgatgagcagcccggccctcccggccttcctgctctgcagcacgctgctggtcatcaagatgtacgtggtggccatcatcacgggccaagtgaggctgcggaagaaggcctttgccaaccccgaggatgccctgagacacggaggcccccagtattgcaggagcgaccccgacgtggaacgctgcctcagggcccaccggaacgacatggagaccatctaccccttccttttcctgggcttcgtctactcctttctgggtcctaacccttttgtcgcctggatgcacttcctggtcttcctcgtgggccgtgtggcacacaccgtggcctacctggggaagctgcgggcacccatccgctccgtgacctacaccctggcccagctcccctgcgcctccatggctctgcagatcctctgggaagcggcccgccacctgtgaccagcagctgatgcctccttggccaccagaccatgggccaagagccgccgtggctatacctggggacttgatgttccttccagattgtggtgggccctgagtcctggtttcctggcagcctgctgcgcgtgtgggtctctgggcacagtgggcctgtgtgtgtgcccgtgtgtgtgtatgtgtgtgtgtatgtttcttagccccttggattcctgcacgaagtggctgatgggaaccatttcaagacagattgtgaagattgatagaaaatccttcagctaaagtaacagagcatcaaaaacatcactccctctccctccctaacagtgaaaagagagaagggagactctatttaagattcccaaacctaatgatcatctgaatcccgggctaagaatgcagacttttcagactgaccccagaaattctggcccagccaatctagaggcaagcctggccatctgtatttttttttttccaagacagagtcttgctctgttgcccaagctggagtgaagtggtacaatctggctcactgcagcctccgcctcccgggttcaagcgattctcccgcctcagcctcctgagtagctgggattacaggcgcgtatcaccatacccagctaatttttgtatttttagtagagacgggttcaccatgttgcccaggagggtctcgaactcctggcctcaagtgatccaccggcctcggcctcccaaagtgctgggatgacaggcatgaatcactgtgctcagccaccatctggagttttaaaaggctcccatgtgagtccctgtgatggccaggccaggggacccctgccagttctctgtggaagcaaggctggggtcttgggttcctgtatggtggaagctgggtgagccaaggacagggctggctcctctgcccccgctgacgcttcccttgccgttggctttggatgtctttgctgcagtcttctctctggctcaggtgtgggtgggaggggcccacaggaagctcagccttctcctcccaaggtttgagtccctccaaagggcagtgggtggaggaccgggagctttgggtgaccagccactcaaaggaactttctggtcccttcagtatcttcaaggtttggaaactgcaaatgtccccttgatggggaatccgtgtgtgtgtgtgtgtgtgtgtgtgtgtgtgtgtgtgtgtgtgtgtgttttctcctagacccgtgacctgagatgtgtgatttttagtcattaaatggaagtgtctgccagctgggcccagca

SEQ ID NO: 11 - DNA, 3860 bp, Homo sapiens
gtgaacgggcagaagcagggccatgcccaagccacccccaagatccccctgaacctgcacctccatcacgacccattcaggagcctccaggagcccagacaccagccccccaccatgggcagcaaggagcgcttccactggcagagccacaacgtgaagcagagcggcgtggatgacatggtgcttcttccccagatcaccgaagacgccattgccgccaacctccggaagcgcttcatggacgactacatcttcacctacatcggctctgtgctcatctctgtaaaccccttcaagcagatgccctacttcaccgaccgtgagatcgacctctatcagggcgcggcccagtatgagaatcccccgcacatctacgccctcacggacaacatgtaccggaacatgcttatcgactgtgagaaccagtgtgtcatcattagtggagagagtggagctgggaagacagtggcagccaaatatatcatgggctacatctccaaggtgtctggcggaggcgagaaggtccagcacgtcaaagatatcatcctgcagtccaacccgctgctcgaggccttcggcaacgccaagactgtgcgcaacaacaattccagccgctttggcaagtactttgagatccagttcagccgaggtggggagccagatgggggcaagatctccaacttcttgctggagaagtcccgcgtggtcatgcaaaatgaaaatgagaggaacttccacatctactaccagctgctggaaggggcctcccaggagcaaaggcagaacctgggcctcatgacaccggactactattactacctcaaccaatcggacacctaccaggtggacggcacggacgacagaagcgactttggtgagactctgagtgctatgcaggttattgggatcccgcccagcatccagcagctggtcctgcagctcgtggcggggatcttgcacctggggaacatcagtttctgtgaagacgggaattacgcccgagtggagagtgtggacctcctggcctttcccgcctacctgctgggcattgacagcgggcgactgcaggagaagctgaccagccgcaagatggacagccgctggggcgggcgcagcgagtccatcaatgtgaccctcaacgtggagcaggcagcctacacccgtgatgccctggccaaggggctctatgcccgcctcttcgacttcctcgtggaggccatcaaccgtgctatgcagaaaccccaggaagagtacagcatcggtgtgctggacatttacggcttcgagatcttccagaaaaatggcttcgagcagttttgcatcaacttcgtcaatgagaagctgcagcaaatctttatcgaacttaccctgaaggccgagcaggaggagtatgtgcaggaaggcatccgctggactccaatccagtacttcaacaacaaggtcgtctgtgacctcatcgaaaacaagctgagccccccaggcatcatgagcgtcttggacgacgtgtgcgccaccatgcacgccacgggcgggggagcagaccagacactgctgcagaagctgcaggcggctgtggggacccacgagcatttcaacagctggagcgccggcttcgtcatccaccactacgctggcaaggtctcctacgacgtcagcggcttctgcgagaggaaccgagacgttctcttctccgacctcatagagctgatgcagaccagtgagcaggccttcctccggatgctcttccccgagaagctggatggagacaagaaggggcgccccagcaccgccggctccaagatcaagaaacaagccaacgacctggtggccacactgatgaggtgcacaccccactacatccgctgcatcaaacccaacgagaccaagaggccccgagactgggaggagaacagagtcaagcaccaggtggaatacctgggcctgaaggagaacatcagggtgcgcagagccggcttcgcctaccgccgccagttcgccaaattcctgcagaggtatgccattctgacccccgagacgtggccgcggtggcgtggggacgaacgccagggcgtccagcacctgcttcgggcggtcaacatggagcccgaccagtaccagatggggagcaccaaggtctttgtcaagaacccagagtcgcttttcctcctggaggaggtgcgagagcgaaagttcgatggctttgcccgaaccatccagaaggcctggcggcgccacgtggctgtccggaagtacgaggagatgcgggaggaagcttccaacatcctgctgaacaagaaggagcggaggcgcaacagcatcaatcggaacttcgtcggggactacctggggctggaggagcggcccgagctgcgtcagttcctgggcaagagggagcgggtggacttcgccgattcggtcaccaagtacgaccgccgcttcaagcccatcaagcgggacttgatcctgacgcccaagtgtgtgtatgtgattgggcgagagaaagtgaagaagggacctgagaagggccaggtgtgtgaagtcttgaagaagaaagtggacatccaggctctgcggggagtctccctcagcacgcgacaggacgacttcttcatcctccaagaggatgccgccgacagcttcctggagagcgtcttcaagaccgagtttgtcagccttctgtgcaagcgcttcgaggaggcgacgcggaggcccctgcccctcaccttcagcgacacactacagtttcgggtgaagaaggagggctggggcggtggcggcacccgcagcgtcaccttctcccgcggcttcggcgacttggcagtgctcaaggttggcggtcggaccctcacggtcagcgtgggcgatgggctgcccaagagctccaagcctacgcggaagggaatggccaagggaaaacctcggaggtcgtcccaagcccctacccgggcggcccctgcgccccccagaggcatggatcgcaatggggtgcccccctctgccagagggggccccctgcccctggagatcatgtctggagggggcacccacaggcctccccggggccctccgtccacatccctgggagccagcagacgaccccgggcacgtccgccctcagagcacaacacagaattcctcaacgtgcctgaccagggcatggccggcatgcagaggaagcgcagcgtggggcaacggccagtgcctggtgtgggccgacccaagccccagcctcggacacatggtcccaggtgccgggccctataccagtacgtgggccaagatgtggacgagctgagcttcaacgtgaacgaggtcattgagatcctcatggaagatccctcgggctggtggaagggccggcttcacggccaggagggccttttcccaggaaactacgtggagaagatctgagctgggccctgggatactgccttctctttcgcccgcctatctgcctgccggcctggtggggagccaggccctgccaatgagagcctcgtttacctgggctgcaatagcctaaaagtccagtcctttggcctccagtcctgcccaggccctgggtcaccaggtcactgctgcagcccccgcccctgggccctggtcttcctccaacatcacacctgctgcccattctccatttctgtgtgtgtcaaaggggactaacagcagaatctacctcccaactgccatgtgattaagaaatgggtcttgagtcctgtgctgttggcaaagtgccaggcacagttggggaggggggggtccttaacaagcgtgactttgctcattctgtcatcactaaggcaataaacctttgccaggtgaaaaaaaaaaaaaaaaaaaaaaaaaaaaaaaaaaaa

SEQ ID NO: 12 - DNA, 1546 bp, Homo sapiens
taactgtccaccagaaaggactgctctttgggtgagttgaacttcttccattatagaaagaattgaaggctgagaaactcagcctctatcatgtggaacagctctgacgccaacttctcctgctaccatgagtctgtgctgggctatcgttatgttgcagttagctggggggtggtggtggctgtgacaggcaccgtgggcaatgtgctcaccctactggccttggccatccagcccaagctccgtacccgattcaacctgctcatagccaacctcacactggctgatctcctctactgcacgctccttcagcccttctctgtggacacctacctccacctgcactggcgcaccggtgccaccttctgcagggtatttgggctcctcctttttgcctccaattctgtctccatcctgaccctctgcctcatcgcactgggacgctacctcctcattgcccaccctaagctttttccccaagttttcagtgccaaggggatagtgctggcactggtgagcacctgggttgtgggcgtggccagctttgctcccctctggcctatttatatcctggtacctgtagtctgcacctgcagctttgaccgcatccgaggccggccttacaccaccatcctcatgggcatctactttgtgcttgggctcagcagtgttggcatcttctattgcctcatccaccgccaggtcaaacgagcagcacaggcactggaccaatacaagttgcgacaggcaagcatccactccaaccatgtggccaggactgatgaggccatgcctggtcgtttccaggagctggacagcaggttagcatcaggaggacccagtgaggggatttcatctgagccagtcagtgctgccaccacccagaccctggaaggggactcatcagaagtgggagaccagatcaacagcaagagagctaagcagatggcagagaaaagccctccagaagcatctgccaaagcccagccaattaaaggagccagaagagctccggattcttcatcggaatttgggaaggtgactcgaatgtgttttgctgtgttcctctgctttgccctgagctacatccccttcttgctgctcaacattctggatgccagagtccaggctccccgggtggtccacatgcttgctgccaacctcacctggctcaatggttgcatcaaccctgtgctctatgcagccatgaaccgccaattccgccaagcatatggctccattttaaaaagagggccccggagtttccataggctccattagaactgtgaccctagtcaccagaattcaggactgtctcctccaggaccaaagtggccaggtaataggagaataggtgaaataacacatgtgggcattttcacaacaatctctccccagcctcccaaatcaagtctctccatcacttgatcaatgtttcagccctagactgcccaaggagtattattaattattaataaatgaattctgtgcttttaaaaaaaaaaaaataaaaaaagaaaaaaaaaaaaaaaaaaaaaaaaaaaaa

SEQ ID NO: 13 - DNA, 355 bp, Homo sapiens
gctttttaaatgacccaggcgtgtgtaataatataatgaataaccatagagcagtgcctttaaattagctataggaaggaaatagtcttttcaagtttctgaacaatatatttctcttagttggcacctcacaaatactagatcatgtcagacgctgctggttaatagctgcaggaaggcatgttgtgcagtggatattgctcatggaagtgtgtgaaatcatagtaagctttgttctccctgctaagacttgctatgtatatttccatcattgtttcatgtaaactgaaccattgtggtaaacttttggagttgatatggaatcactttaatgctgttttcacaaataaaagtt

SEQ ID NO: 14 - DNA, 1630 bp, Homo sapiens
tataaacaacattcaaataaccttggaccttggtgaaatgacttgtggtggccagaatggtgcaacaagatgttatttgcaagtttttttaagacacaaatatctcagatactaataatgagaataaagactgttgaatatgaaattaaagccaagcaataatgtgccaaaaagaggcagttataccagcaaatgcatctattatgggcacaccattatataatgatggtttgctttatgaagactgactgtaacccacaggataaaataagcaaaggcatagtttctgctttcttcctggaaaaacttgtttagaagcttcataaagaggtacagcactaatgagcattagtcaggatacagttggcatctatgtttttatgtgagcccagagggaagaggagccactcaaagtcttgctggcttaaaactcaagacagctgcaaccagaagttttgttgaaatggagactttaaacttatggtaattactctttctggacactagcatgtagaaagcaattcagttaactctgcccagaggattaccagctttagctgtgaaaaaatgggctcccggatgtaaaatcactaaaacatgagatcttgtatccaaagaggcttcaaatgatgccttacagaaaacgatgctccagatgggcacttctaaatgctaactcttcatcaagtatctttctggattcaagctcaaaattaattggctgcaaaatagtaggaataaaaatcacatattttacactttagaaaaggatattgatgatcaacctgcatggtgataattatgatgagataccccagtgatttaatgatgttagaaagaattaaatgggagagaattgctaacagctttcttgatctcttaactatggagatgtcattcatttatttctggggtgaaaattatagcttgctttttgacattgctgctagtattgttctttgttgctttaaaaattgtctctctttagaaaaactcttgagcagttaaacagttttttttctgattcatatcattgcttttaataacatgtaaaggctgtgtgtagagcaaactatataaaatgagtagaaagggcttactcatgttaattggcatccttgatgattttagttgagattccttaacatttattttagatcacatctttacgtaacttatttttcctaatgttttccatcgtgtcttaaaatgatgctggtatatcaggagattgcagtattatagtcatactccccaatccctagaggagaggaaagactaattcttgttttaagggcccctggagataccttttattaaggttgaaaaaggtcaacacagcctgaaaataagaaaaatatatactagcaattactaattttctaaatgtgtgtatctctgctgtactaatgtgtgaacaatatgtcgtgcataatactgtagctggtcgtggtatgtcaatacattctgtgagtgtgtacagtctgagtgatcagttttctatttttatgtgtaaaaaaaataacttgtcgtatcccatttaaaggccaatttctgtattcaggcaggcatatgtacatacatgaataaagccaacaaaagtgtgcacatgtaaaaaaaaaaaaaaaaa

SEQ ID NO: 15 - DNA, 1123 bp, Homo sapiens
agcggtcgggtccgggcgcccgcgcagaatcagctgtctgagctgcccaggcggcgggggagcagcgagcgggcttcagcgagccgcaggaggcacaggcctgtcctgggtccccgcaggtctgcgcgtctgttgttcccagcgctctgagaggcctgaaaaggaagagcaacctgtccagaatccccgcaggaaaggaaaaggaggggaaatctcgacatggaaaaactcttcaatgaaaatgaaggaatgccttcgaatcaaggaaagatagacaatgaagaacagccaccgcacgagggaaagccagaagtagcttgtattctggaagacaagaagttagaaaacgagggaaacacagaaaacacgggcaagagagttgaggaaccgttaaaggataaagaaaagccagagagtgcgggaaaggcaaaaggagaaggaaagtcagagaggaagggaaagtcagagatgcagggaggatcaaagacagagggaaagccagagagagggggaagggcagagggtgaaggagagccagacagtgaaagagagccagagagtgagggagagccagaaagtgaaacaagggctgcaggaaagcgcccagctgaggatgatatacccaggaaagccaaaagaaaaaccaacaaggggctggctcagtacctcaagcaatataaggaagccatacatgatatgaatttcagcaatgaggacatgataagagaatttgacaacatggctagggtggaggataaaaggagaaaaagcaaacagaaattgggggcgtttttgtggatgcaaagaaatttacaggaccccttctatcctaggggtccaagggaattcaggggtggctgcagggccccacgaagggacactgaagacattccttatgtgtagtgtccctggcaggcatttgtcaggccatatgttttaaccttatggtaatactttgctttagtcgttcctcctgctaccagtagcgttttgacccacctgccagtgtttgcttgctctatgtttcagtagcagattttcacacatgtgcattgcagagacgtcatgattcgtggaaaaataaagcagcttataatatcaaaaaaaaaaaaaaaaaaaaaaa

SEQ ID NO: 16 - DNA, 3080 bp, Homo sapiens
agaggtgcgcgctgcgcgtgggatcagcccggcgccgacgggtggctccgaggagctcgctccttcctcgcccccgccccctcgccgcgcggggccagcccggccgctcctcccctgggtgggtccctgctccttttctggcagggtctatttgcatagaggaaactgcccaaagtggccgctgtggaggagctggctgcggcgaagggggcgtgcgcggcgatccgctgctacccggaggctaacccccgcgcccggcggacctcgtgcctcgggctgtcccgcctgctcctctcgcacccagcctctgccccagcagcaccgccccctcggagagtccacgcgcgacgaacgcgccatgggcccaggcgagcgcgccggtggcggcggcgacgcggggaagggcaatgcggcgggcggcggcggcggagggcgctcggcgacgacggccgggtcccgggcggtgagcgcgctgtgcctgctgctctccgtgggctcggcggctgcctgcctgctgctgggtgtccaggcggccgcgctgcagggccgggtggcggcgctcgaggaggagcgggagctgctgcggcgcgcggggccgccaggcgccctggacgcctgggccgagccgcacctggagcgcctgctgcgggagaagttggacggactagcgaagatccggactgctcgggaagctccatccgaatgtgtctgccccccagggccccctggacggcgcggcaagcctgggagaagaggcgaccctggtcctccagggcaatcaggacgagatggctacccgggacccctgggtttggatggcaagcccggacttccaggcccgaaaggggaaaagggtgcaccaggagactttggcccccggggagaccaaggacaagatggagctgctgggcctccggggccccctggacctcctggggcccggggccctcctggcgacactgggaaagatggccccaggggagcacaaggcccagcgggccccaaaggagagcccggacaagacggcgagatgggcccaaagggacccccagggcccaagggtgagcctggagtacctggaaagaagggcgacgatgggacaccaagccagcctggaccaccagggcccaagggcgagccagggagcatggggcctcggggagagaacggtgtggacggtgccccaggaccgaagggggagcctggccaccgaggcacggatggagctgcagggccccggggtgccccaggcctcaagggcgagcagggagacacagtggtgatcgactatgatggcaggatcttggatgccctcaaggggcctcccggaccacaggggcccccagggccaccagggatccctggagccaagggcgagcttggattgcccggtgccccaggaatcgatggagagaagggccccaaaggacagaaaggagacccaggagagcctgggccagcaggactcaaaggggaagcaggcgagatgggcttgtccggcctcccgggcgctgacggcctcaagggggagaagggggagtcggcgtctgacagcctacaggagagcctggctcagctcatagtggagccagggccccctggcccccctggccccccaggcccgatgggcctccagggaatccagggtcccaagggcttggatggagcaaagggagagaagggtgcgtcgggtgagagaggccccagcggcctgcctgggccagttggcccaccgggccttattgggctgccaggaaccaaaggagagaagggcagacccggggagccaggactagatggtttccctggaccccgaggagagaaaggtgatcggagcgagcgtggagagaagggagaacgaggggtccccggccggaaaggagtgaagggccagaagggcgagccgggaccaccaggcctggaccagccgtgtcccgtgggccccgacgggctgcctgtgcctggctgctggcataagtgacccacaggcccagctcacacctgtacagatccgtgtggacatttttaatttttgtaaaaacaaaacagtaatatattgatcttttttcatggaatgcgctacctgtggccttttaacattcaagagtatgcccacccagccccaaagccaccggcatgtgaagctgccggaaagtggacaggccagaccagggagatgtgtacctgaggggcacccttgggcctgggctttcccaggaaggagatgaaggtagaagcacctggctcgggcaaggctagaaagatgctacgttgggccttcagtcacctgatcagcagagagactctcagctgtggtactgccctgtaagaacctgcccccgcaaaactctggagtccctgggacacaccctatccaagaagacccaggggtggaacagcggctgctgttgctcctggcctcatcagcctccaaactcaaccacaaccagctgcctctgcagttggacaagacttggcccccggacaagactcgcccagcacttgcggctgggcccggggagcagtgagtggaaatcccccacgagggtctagctctaccacattcaggaggcctcaggaggccagcctgccatgagagcacatgtcctctggccaggagtagtggctgagctctgtgatcgctgtgatgtggacccagctccagggagcagagtgtcgaggatggaggggcccagcctggactgactgctacttcctgtctctgtttccattatcacccagagagggacaagataggacatggcctggaccagggaggcaggcctcccactcagagtctgggtctcactggccccaagtctcccacccagaactctggccaaaaatggctctctaggtgggctgtgcaggcaaagcaaagctcagggctggttcccagctggcctgagcagggggcctgccaccagacccacccacgctctgacgagaggcttttccacctccagcaagtgttcccagcaaccagctccatcctggctgcttgccttccatttccgtgtagatggagatcactgtgtgtaataaaccacaagtgcgtgtctgaaaaaaaaaaaaaaaaa

SEQ ID NO: 17 - DNA, 6330 bp, Homo sapiens
tgacgcccccgaacccagctgcagaagctgccgccacctccaatgcacaaggtgtctcatctgaaaagaaacctgagccccagggaggcggcgcggagcgaccctggcagagctggcgcaaacagggcgagaggtcgctgggcagcgttcgaggaccagagggagctcggccacagaagaccccagtgatctgatcccgggatcccggctccaagctctcctcgcattttacagatttcacccccgcgactatctccccaaaacggagcctttatatcaagagaaggtgcgggagctggggcaaccaggactttctcgggcacccaagatgcgctccattaggaagaggtggacgatctgcacaataagtctgctcctgatcttttataagacaaaagaaatagcaagaactgaggagcaccaggagacgcaactcatcggagatggtgaattgtctttgagtcggtcacttgtcaatagctctgataaaatcattcgaaaggctggctcttcaatcttccagcacaatgtagaaggttggaaaatcaattcctctttggtcctagagataaggaagaacatacttcgtttcttagatgcagaacgagatgtgtcagtggtcaagagcagttttaagcctggtgatgtcatacactatgtgcttgacaggcgccggacactaaacatttctcatgatctacatagcctcctacctgaagtttcaccaatgaagaatcgcaggtttaagacctgtgcagttgttggaaattctggcattctgttagacagtgaatgtggaaaggagattgacagtcacaattttgtaataaggtgtaatctagctcctgtggtggagtttgctgcagatgtgggaactaaatcagattttattaccatgaatccatcagttgtacaaagagcatttggaggctttcgaaatgagagtgacagagaaaaatttgtgcatagactttccatgctgaatgacagtgtcctttggattcctgctttcatggtcaaaggaggagagaagcacgtggagtgggttaatgcattaatccttaagaataaactgaaagtgcgaactgcctatccgtcattgagacttattcatgctgtcagaggttactggctgaccaacaaagttcctatcaaaagacccagcacaggtcttctcatgtatacacttgccacaagattctgtgatgaaattcacctgtatggattctggcccttccctaaggatttaaatggaaaagcggtcaaatatcattattatgatgacttaaaatataggtacttttccaatgcaagccctcacagaatgccattagaattcaaaacattaaatgtgctacataatagaggagctctaaaactgacaacaggaaagtgtgtaaagcaataaagcacattttgaaacaaacaatatgcacttcttttctgaagatgcttccgaagatttgaaaataggatccaaaacacggctgggtttcagcatccaccaatgaactgaaaggtgaataaaggacgttcatgagaaatcgactaccagctgatgaaatacctgcaaagtgctctaaaaattaaatattttgactttaagggtcctagtaagtgccacttccactaagaatacagtttgaatgtataatcagtagtgtttacaagatccaacagtgcactcatcattagttaacaaagcaaatatgttcatcactgtcaggctgcccacagcaacaccaagcatattagaagaggaaccccaggaacgcaactcagaccttgggaaattaaaccatccttgtcagcagaagccaagatggaagcagtttgagcaatgaaatccgtaagattaaacaactcaagtaaatgcttcagtcaggactctgagtctgatcatgaattttatgttttaatttatgttttttttttgtcttctggaatctcttttggtttggatattgggatgcttagaaatcctttctgagatgcatatgagtgaggaaataaactttaagtaattatttttaaagttcttatactttttaaaagctatcacacaaagacttttttttttttttttgtctcgctctgttgcccaggctggagtacagtggcgcgatctcagctcactgcaagctccgcctcccaagttcactccattctcctgcctcagcctccggagtagctgggactgcaggcgcctgccaccacgcctggctaattttttgtatttttagtggagacggattttcaccgtgttagccaggatggtctcaatctcctgacctcgtgatccacccgccttggcctcccaaagtgctgggattacaggcgtgagccaccgtgcctggccgacatttttaaaaaagttttattttgcacggctctaaacctccatgttattttccagtggtgtagaaggtaccagctaaagtgaaccactatgtaatattaggccattctaaaggaaagatgttccatgtcatcagagatggtaaaataggccgggaaaaaaaaatctttggtaccaaagattacacttgtgtttctacacagcaaaccatttttctttcatgaaaataatatattattaacatgaatatattattttgctattaatgtgaaagttgtctctaaatattttttaattttcaaactcatactttattttcatttgaaatgtttttcacaccttttgcattacataataattttgtggaagcattttgccctttagaataaatattagattgatatagctgaaatgtgacttccagttctttgatattccccttgttattcaaatagaaatatggaaatgctttatatattactgttaaatttcttagtgcagaaataacattattaatagagtattgttttcaaaacagagatgattaatttcaagaggtttaacagtgaaattgtgtcaatattttgcatttaaaatgaatttaattgaccgatattttctgtagttaaatttagtcacaatatcacatatgttcttcaagaaacacatgaaattattaataaagtaattaaaaaatttttaatgtataacagaattgaccaataggccagttttctggtaacttatgatagtagattgtttctttagaaactgggcagaagctctgcattctcacttgtactttgatttcttatttcttgggcaggcaatttgaggaaagaagaaatggcatggggaatatatatgttttgtttcttagggaaaacagtctgagaaatgaataaaaagcatgaagtacgtgtgtgtgtgtgtgtgttaccatggaaaaggatattcacagtagtacagttctcaatatttttaattagatgtcatatttttttaatatagtaaaaccttgggatatagaatattacatcttttgagaatgtatgtgtctctaagtaagtaaaatctaatgcgtataggagactgatagctaaaaatgaatggaacattaatgtacttttataattaaacctcttatctatcagaaattgtaagagaatagatacatgttttgaatgtaaagttgaaaagtctggtttacttaataaattgaaagtgatttataaaatctaaatttggactacttgcaaatgataagctattctagtagcctttagtttaaatccaacagaaatctagaagtcacaagcaaatatcttaaaggtaaaatccatctgggcactcatttaaagtatatcttaaaaaagcagcagcaaggtaccttgccatttttagcatattttcttcctttttcttttttcttttttttttttttttgagatggagtctcactctgtcacacaggctggaatgcagtgatgccatctcagctcactgcaacctccacctcctgggttcaagtgattctcgtgcctcagcctcccaagtagctggggttacaggcgcccaccaccacactcggctaattttgtgtttttagtagagacaaagtttcaccatgttggccaggctggtcttgaacttcctgacctcaggttatccacccacctcagcctcccaaagtgctgggattacaggtgtgagccaccgcagccggaccatttttagtatattttcagtaaatacatttaaacaatgttaaggccacagcacacatatctcagccattcattgttctgtgcattgatgtttatctcatagatgcattgagtagtgcctttttagctttttcacattactttgtcaccatatcctttgtgttctctaaatacattgcccacttccaaaaatgttcagcatgaaaaaaagggcttcagtgtcgattgagattgcttttgttcatctcagggatttcaatagtcaagaatgaattcagttaaaggtatttagggttcaaagaagacaaactgtacaagcccatttcattccttgttgtatacctttccatctgccctcccattttaactatctactgtggcctttttatggaaacagagcaagatcaatgaaggctaatggcaagaataagaaaaagagttgagatttaaccaatagcggagcataaaggatcatgacaaaatcaaattataaaagcatacttgaaataggtggagctttttcttttgaaaatatatattcacaattttaatattttaatttattttttactatttaaccctgtacttggcaatgctcaggcagctgattgtgaaatattcttgtcctttacagaacatggttgttattgtgctgttgacatgaatagaccatggaaacattttcatcattattattcagcctgtgctgtagttaatgttaagttgctgaaataaaaagtgagcaagtaatagattttcttggcaaatctaatgattcagcccacaggactgttgaaactactgcggaagtttttctatctgaaagaaggtgctgggcattcaaatgtgttcatgtattgtatatcatatgaattgtatatcaattactaatgggaatttctacatatatgcttacaaaagcaatttatttaagtaatgctaggggtagtgtacataccaattagttattcagctactatacagaaaaaggatgaacaaattaatttatttctaattgagccagttagacataatgcatataacgtgatatttggttcatgaaagagttgttttcatgtggttattgtagggagtatatataattgtggaaggggtatgggaagagttgtgtatagttagttgttatctctacaagtttgaaagttttcccatcaaacattatcaatataccaatgttttaaaaattgagtgagggttattatttgtatttgatgaaagaaaatccaaataaagcccacctagaaatagatattttattatatatgtgctatagatatacctatatagtacaaatagacatgtgtgatgcatatatacaatgttatatatgtgtatatgtctgtatacacactgagtctgtaatatgtatacactaaatttgtgttatgctaacatcttcagggtctgcactgtgaactcccctggagataagtaagtccactttagaataaagaagttcttttgagacttcagttactaacgtgctttaagaggtatctactttataactgaattctatgtcgttcatacgtagagttacagtaagggtctagtatgtccaaatcttaataataaagaagaaaagtaaaggcttcaagctagcaatgtattcgaattacagttttcagattgtggctccaggccttgtgtttctcatttaagtagcaccttttaataaaaaccgtttctttgtgtaggcaaaagcacaagtgtttcaaatgtaaatagcaggaaaaaaaaagagtttacagagatagcattgctgcacagaataattgctactgagtatttcttatagaatttgtggaactgaaagatgaggtttattctgtcaagttcaagttcattctgttcaacactgttttcttattgtttgtgtatagcaaccgggtattattgttttatcatttgtaaaattgtaaaataaattaatccctttttttcactgtttctcttatctcatatatccaagcccttggttatactttgtatgtcaatgttaggtgatcatttttaacaagctttggcttgtgctttgcttttccactccccttagccctagtggttggcaattaggcaaaccatttatttttaagtgtatacatgggaatatgaacaatgtcaaaaaccccatgaatattaggaaatccttaacgatattttgtgtagcacattctgtttgcggttgagggaataaagtatttcacaagtgaaaaaaaaaaa

SEQ ID NO: 18 - DNA, 432 bp, Homo sapiens
ttttttttttttttttttaacaggagttatttctgattttatttataatataaaaatgttcaagtgtcaacagtcaggtgttcagacatttcaggacaggattcccatctgtttctgtttgggattttttttttttttttaaacaattacctttttgacaaattagcagtggacccagtttttgggggtgggagggcaggactggagacgagtggatgtcataggtgggttgggggctaggaggcagcctgtgagaaggaaatggtgttactttattgctaaaaggggaatacactgtcgagtggctcttctcggtcccagcgtgaccatgcatccaatctaaagaatctgaaatgcaaaggacatgcaggtgtaaaatagaaaagacgacctgtaaacgaaggtgctgcaaaggacggaggggcgtcctgg

SEQ ID NO: 19 - DNA, 3056 bp, Homo sapiens
gacacatgatgctgtgaacgtcagggtgctcgccagggaagggccctacccagagggacagaaagaaagccaggaggggtagagtttgaagagaagatcatgttctccctgaagacgcttccatttctgctcttactccatgtgcagatttccaaggcctttcctgtatcttctaaagagaaaaatacaaaaactgttcaggactacctggaaaagttctaccaattaccaagcaaccagtatcagtctacaaggaagaatggcactaatgtgatcgttgaaaagcttaaagaaatgcagcgattttttgggttgaatgtgacggggaagccaaatgaggaaactctggacatgatgaaaaagcctcgctgtggagtgcctgacagtggtggttttatgttaaccccaggaaaccccaagtgggaacgcactaacttgacctacaggattcgaaactataccccacagctgtcagaggctgaggtagaaagagctatcaaggatgcctttgaactctggagtgttgcatcacctctcatcttcaccaggatctcacagggagaggcagatatcaacattgctttttaccaaagagatcacggtgacaattctccatttgatggacccaatggaatccttgctcatgcctttcagccaggccaaggtattggaggagatgctcattttgatgccgaagaaacatggaccaacacctccgcaaattacaacttgtttcttgttgctgctcatgaatttggccattctttggggctcgctcactcctctgaccctggtgccttgatgtatcccaactatgctttcagggaaaccagcaactactcactccctcaagatgacatcgatggcattcaggccatctatggactttcaagcaaccctatccaacctactggaccaagcacacccaaaccctgtgaccccagtttgacatttgatgctatcaccacactccgtggagaaatacttttctttaaagacaggtacttctggagaaggcatcctcagctacaaagagtcgaaatgaattttatttctctattctggccatcccttccaactggtatacaggctgcttatgaagattttgacagagacctcattttcctatttaaaggcaaccaatactgggctctgagtggctatgatattctgcaaggttatcccaaggatatatcaaactatggcttccccagcagcgtccaagcaattgacgcagctgttttctacagaagtaaaacatacttctttgtaaatgaccaattctggagatatgataaccaaagacaattcatggagccaggttatcccaaaagcatatcaggtgcctttccaggaatagagagtaaagttgatgcagttttccagcaagaacatttcttccatgtcttcagtggaccaagatattacgcatttgatcttattgctcagagagttaccagagttgcaagaggcaataaatggcttaactgtagatatggctgaagcaaaatcaaatgtggctgtatccactttcagaatgttgaagggaagttcagcaagcattttcgttacattgtgtcctgcttatacttttctcaatattaagtcattgtttcccatcactgtatccattctacctgtcctccgtgaaaatatgtttggaatattccactatttgcagaggcttattcagttcttacacattccatcttacattagtgattccatcaaagagaaggaaagtaagcctttttgtcacctcaatatttactatttcaatacttacatatctgacttctaggatttattgttatattacttgcctatctgacttcatacatccctcagtttcttaaaatgtcctatgtatatcttctacatgcaatttagaactagattttggttagaagtaaggattataaacaacctagacagtacccttggcctttacagaaaatatggtgctgttttctacccttggaaagaaatgtagatgatatgtttcgtgggttgaattgtgtcccccataaaagatatgttgaagttctaaccccaggtacccatgaatgtgagcttaccagggtctttgcagatgtaattagttaagttaaggtgagatcacactgaattagggtgggctctaaatccattatgactgttgttcttataagaagaagagaggcatagtcacctaggggaggaggccgtatgaagacagaggcagagattggagtgacgcatctccaagccaaggaattccaaggactgtaagccaccagtagaagctttgaagaggcaaggaaggattccctccaatagccttcaagtgtgaccctgctgacacctgcagaattcggacttctatcctccaaaaccgtgagggaataaatttcctttgttttaagccaccaactttgcaatactttgttacagcaaccctagacatgaggtactagacacagtacatctacacatatgaaaatgaatcaacacagaatgcagaagtagaacccttgctaaggactactgggcatcttcccaggacagcagccaaaagagaaccaccacttcctctcctgcctcctccttgctctctcctagagtccaaacccaaatgggccagttggatctgatgttcgtcagttctttacttctatttcctggggtactcaggagggcacacactatagataacttgggttagctgcataaaattcaatgtctcattaagttgcattaaactgagcttagatgtgtaagtttgctaacggatgggtttttttgttaagaactataggatttatgggaccaagtctagcgagtccagatatcaaaatcattataatgttatatttgctgttattagaatataatatagcttattatacaataaatatgtagactgtaaaatatatttctcactagtacctcctattttctttctctgttgaagtttttaaatcccacagataattaaattggcacctttatgcttgttcaaaaattaaaataatctattaaataagttcaaattaaagatttttacttcaaatgac

SEQ ID NO: 20 - DNA, 2823 bp, Homo sapiens
aagtgggaggagactttgcaaatagcaatcttggggcaggggccattttggaagcatgttgcgaggctccgcttcttctacaagtatggagaaggcaaaaggcaaggagtggacctccacagagaagtcgagggaagaggatcagcaggcttctaatcaaccaaattcaattgctttgccaggaacatcagcaaagagaaccaaagaaaaaatgtctatcaaaggcagtaaagtgctctgccctaagaaaaaggcagagcacactgacaaccccagacctcagaagaagataccaatccctccattaccttctaaactgccacctgttaatctgattcaccgggacattctgcgggcctggtgccaacaattgaagctgagctccaaaggccagaaattggatgcatataagcgcctgtgtgcctttgcctacccaaatcaaaaggattttcctagcacagcaaaagaggccaaaatccggaaatcattgcaaaaaaaattaaaggtggaaaagggggaaacgtccctgcaaagttctgagacacatcctcctgaagtggctcttcctcctgtgggggagccgcctgccctggaaaattccactgctctccttgagggagttaatacagttgtggtgacaacttctgccccagaggctttgctggcctcctgggcgagaatttcagccagggcgaggacaccagaggcagtggaatctccacaagaggcctctggtgtcaggtggtgtgtggtccatgggaaaagtctccctgcagacacagatggttgggttcacctgcagtttcatgctggtcaagcctgggttccagaaaagcaagaagggagagtgagtgcactcttcttgcttcctgcctccaattttccacccccgcaccttgaagacaatatgttgtgccccaaatgtgttcacaggaacaaggtcttaataaaaagcctccaatgggaatagaatatcaggaaaaaggccacatctatggtaattaatggcagaaaagctggagagttggattctgcggtgctgctgacaggtgaactctggtcctctgcacctgtttatgggccatgcagactggtggggtggcagatgttagcctaagacccctagcagtgcctgttgctttgtgagtggagatagagactcttacatttaaaaatggaaaaacatttcacaaattaccataaattgtagttaatatgtagaaaaactcattcatactacttttctaaaatagacatgacttcagcagcagcttttttttgttgtattttgagacagtgtctcactgttgcccaggctggagtgcagtggtgcaatctcagttcagtgcaatctccgcctcctgggttcaaatgattctcctgcctcagcctcctgagtagctaggtacaggcacctgccaccacacccagctaattttttgtatttttagtagagatggggtttcaccatgttggccaggttggtctcaaactcctggactcaagtgatcaccctcctcagcctcccaaaatgctgggactatgggcatgagcccctgcgcctgaccttcaacagctcttttaagtgagttcttcagctaagcattgtgatggacttgagtaaaatggtagttggctcttgtgctcaattttctcttcctctgaacactgactactttaggagctgcttcattccaattgcaatttcataaaacgtaaagtattttaaggcaaagaaaggctgttaattccctccctcccccaaacacatgatttttaatattctaaacaatatttttcaaagttctcttaataacctgagatttctatggtttgactccaggatcaaaacacaagggactttgtattatttcacttataattgttttgtatatttctggagtttaaaatgtttaaggttgcttcccgctcataaatacataatatattgaatttaaaatgtgtttattaaccgattctccataaataaaaataagatgtgtatgtaaaataattcatctgttgtatttagagaaccatattcattgcatgcaaattttattgttagtgttcttaactcaagtaggagtaaaccaaaaagtgtgatttttcttttgtatgactcgtttgttctttattagttggtggtatgggttggatcatttgtttttaaaactacttaggtatgattcacatacaaaaagctgcacatatttaatgtatcctattgtgtaattaatttttaatttttttgtgtacttcctaaacttatagtcctgcgagtctgggaacagatctgtttttcacttatcctgatttaatgacagtttccaacattgttttgttattacaagtaggggatctttttttttgcccgtttaatgaagatactaaaaataatgcactggaaggagtggaagagttggaaaatttgtaaccatcataatacaggtgtaataggtttgggaaagaatcctcaaaaatgttaaagcaagggaggaaagtttgttgagaagcaagatgttcttctctcctgcccgcccccgccgttggttgttggtggtcagaattattgtgtaataaataatagacattttttcttatactatgtgtattgttccttttgtttcctttttaaacttctcccctgctttatttggatgggtcaagtttctgttctgtttccttcctttctattaatttggaaatgtccttggctttacgattctgcttgtagatacttcccctgcttctaacacatttcaataaacttaaatttctctatatacaaaataaattaataattggagtctaccaaaaaaa

SEQ ID NO: 21 - DNA, 1027 bp, Homo sapiens
tctcttctccactatggacagagcctccactgagctgctgcctgcccgccacatacccagctgacatgggcaccgcaggagccatgcagctgtgctgggtgatcctgggcttcctcctgttccgaggccacaactcccagcccacaatgacccagacctctagctctcagggaggccttggcggtctaagtctgaccacagagccagtttcttccaacccaggatacatcccttcctcagaggctaacaggccaagccatctgtccagcactggtaccccaggcgcaggtgtccccagcagtggaagagacggaggcacaagcagagacacatttcaaactgttccccccaattcaaccaccatgagcctgagcatgagggaagatgcgaccatcctgcccagccccacgtcagagactgtgctcactgtggctgcatttggtgttatcagcttcattgtcatcctggtggttgtggtgatcatcctagttggtgtggtcagcctgaggttcaagtgtcggaagagcaaggagtctgaagatccccagaaacctgggagttcagggctgtctgaaagctgctccacagccaatggagagaaagacagcatcacccttatctccatgaagaacatcaacatgaataatggcaaacaaagtctctcagcagagaaggttctttaaaagcaactttgggtccccatgagtccaaggatgatgcagctgccctgtgactacaaggaggaagagatggaattagtagaggcaatgaaccacatgtaaattattttattgtttcatgtctgcttctagatctaaaggacactagcattgccccagatctgggagcaagctaccaacaggggagactctttcctgtatggacagctgctgtggaaatactgcctgcttctcccacctcctcagagccacaggaaagaggaggtgacagagagagagcaaggaaagtgatgaggtggattgatactttctactttgcattaaaattattttctagcctgcagtct

SEQ ID NO: 22 - protein, 158 aa, Homo sapiens
MMQKLLKCSRLVLALALILVLESSVQGYPTRRARYQWVRCNPDSNSANCLEEKGPMFELLPGESNKIPRLRTDLFPKTRIQDLNRIFPLSEDYSGSGFGSGSGSGSGSGSGFLTEMEQDYQLVDESDAFHDNLRSLDRNLPSDSQDLGQHGLEEDFML

SEQ ID NO: 23 - protein, 276 aa, Homo sapiens
MGNSMKSTPAPAERPLPNPEGLDSDFLAVLSDYPSPDISPPIFRRGEKLRVISDEGGWWKAISLSTGRESYIPGICVARVYHGWLFEGLGRDKAEELLQLPDTKVGSFMIRESETKKGFYSLSVRHRQVKHYRIFRLPNNWYYISPRLTFQCLEDLVNHYSEVADGLCCVLTTPCLTQSTAAPAVRASSSPVTLRQKTVDWRRVSRLQEDPEGTENPLGVDESLFSYGLRESIASYLSLTSEDNTSFDRKKKSISLMYGGSKRKSSFFSSPPYFED

SEQ ID NO: 24 - protein, 293 aa, Homo sapiens
MLHRLWASPAAPGKKKEMGNSMKSTPAPAERPLPNPEGLDSDFLAVLSDYPSPDISPPIFRRGEKLRVISDEGGWWKAISLSTGRESYIPGICVARVYHGWLFEGLGRDKAEELLQLPDTKVGSFMIRESETKKGFYSLSVRHRQVKHYRIFRLPNNWYYISPRLTFQCLEDLVNHYSEVADGLCCVLTTPCLTQSTAAPAVRASSSPVTLRQKTVDWRRVSRLQEDPEGTENPLGVDESLFSYGLRESIASYLSLTSEDNTSFDRKKKSISLMYGGSKRKSSFFSSPPYFED

SEQ ID NO: 25 - protein, 316 aa, Homo sapiens
MLSKLGHSPLGGLRARLTFPVCLLYHRLWASPAAPGKKKEMGNSMKSTPAPAERPLPNPEGLDSDFLAVLSDYPSPDISPPIFRRGEKLRVISDEGGWWKAISLSTGRESYIPGICVARVYHGWLFEGLGRDKAEELLQLPDTKVGSFMIRESETKKGFYSLSVRHRQVKHYRIFRLPNNWYYISPRLTFQCLEDLVNHYSEVADGLCCVLTTPCLTQSTAAPAVRASSSPVTLRQKTVDWRRVSRLQEDPEGTENPLGVDESLFSYGLRESIASYLSLTSEDNTSFDRKKKSISLMYGGSKRKSSFFSSPPYFED

SEQ ID NO: 26 - protein, 575 aa, Homo sapiens
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 27 - protein, 499 aa, Homo sapiens
MASNSLFSTVTPCQQNFFWDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 28 - protein, 521 aa, Homo sapiens
MASNSLFSTVTPCQQNFFWDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSTLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 29 - protein, 507 aa, Homo sapiens
MRIPVDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSTLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 30 - protein, 429 aa, Homo sapiens
MASNSIFDSFPTYSPTFIRDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 31 - protein, 415 aa, Homo sapiens
MRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 32 - protein, 1159 aa, Homo sapiens
MYLSICCCFLLWAPALTLKNLNYSVPEEQGAGTVIGNIGRDARLQPGLPPAERGGGGRSKSGSYRVLENSAPHLLDVDADSGLLYTKQRIDRESLCRHNAKCQLSLEVFANDKEICMIKVEIQDINDNAPSFSSDQIEMDISENAAPGTRFPLTSAHDPDAGENGLRTYLLTRDDHGLFGLDVKSRGDGTKFPELVIQKALDREQQNHHTLVLTALDGGEPPRSATVQINVKVIDSNDNSPVFEAPSYLVELPENAPLGTVVIDLNATDADEGPNGEVLYSFSSYVPDRVRELFSIDPKTGLIRVKGNLDYEENGMLEIDVQARDLGPNPIPAHCKVTVKLIDRNDNAPSIGFVSVRQGALSEAAPPGTVIALVRVTDRDSGKNGQLQCRVLGGGGTGGGGGLGGPGGSVPFKLEENYDNFYTVVTDRPLDRETQDEYNVTIVARDGGSPPLNSTKSFAIKILDENDNPPRFTKGLYVLQVHENNIPGEYLGSVLAQDPDLGQNGTVSYSILPSHIGDVSIYTYVSVNPTNGAIYALRSFNFEQTKAFEFKVLAKDSGAPAHLESNATVRVTVLDVNDNAPVIVLPTLQNDTAELQVPRNAGLGYLVSTVRALDSDFGESGRLTYEIVDGNDDHLFEIDPSSGEIRTLHPFWEDVTPVVELVVKVTDHGKPTLSAVAKLIIRSVSGSLPEGVPRVNGEQHHWDMSLPLIVTLSTISIILLAAMITIAVKCKRENKEIRTYNCRIAEYSHPQLGGGKGKKKKINKNDIMLVQSEVEERNAMNVMNVVSSPSLATSPMYFDYQTRLPLSSPRSEVMYLKPASNNLTVPQGHAGCHTSFTGQGTNASETPATRMSIIQTDNFPAEPNYMGSRQQFVQSSSTFKDPERASLRDSGHGDSDQADSDQDTNKGSCCDMSVREALKMKTTSTKSQPLEQEPEECVNCTDECRVLGHSDRCWMPQFPAANQAENADYRTNLFVPTVEANVETETYETVNPTGKKTFCTFGKDKREHTILIANVKPYLKAKRALSPLLQEVPSASSSPTKACIEPCTSTKGSLDGCEAKPGALAEASSQYLPTDSQYLSPSKQPRDPPFMASDQMARVFADVHSRASRDSSEMGAVLEQLDHPNRDLGRESVDAEEVVREIDKLLQDCRGNDPVAVRK

SEQ ID NO: 33 - protein, 1722 aa, Homo sapiens
MRTGWATPRRPAGLLMLLFWFFDLAEPSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNKSLMWFDKTPLSYTHWRAGRPTIKNEKFLAGLSTDGFWDIQTFKVIEEAVYFHQHSILACKIEMVDYKEEYNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDYTAIAIIVATLSILVLMGGLIWFLFQRHRLHLAGFSSVRYAQGVNEDEIMLPSFHD

SEQ ID NO: 34 - protein, 170 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 35 - protein, 749 aa, Homo sapiens
MAHRKLESVGSGMLDHRVRPGPVPHSQEPESEDMELPLEGYVPEGLELAALRPESPAPEEQECHNHSPDGDSSSDYVNNTSEEEDYDEGLPEEEEGITYYIRYCPEDDSYLEGMDCNGEEYLAHSAHPVDTDECQEAVEEWTDSAGPHPHGHEAEGSQDYPDGQLPIPEDEPSVLEAHDQEEDGHYCASKEGYQDYYPEEANGNTGASPYRLRRGDGDLEDQEEDIDQIVAEIKMSLSMTSITSASEASPEHGPEPGPEDSVEACPPIKASCSPSRHEARPKSLNLLPEAKHPGDPQRGFKPKTRTPEERLKWPHEQVCNGLEQPRKQQRSDLNGPVDNNNIPETKKVASFPSFVAVPGPCEPEDLIDGIIFAANYLGSTQLLSERNPSKNIRMMQAQEAVSRVKRMQKAAKIKKKANSEGDAQTLTEVDLFISTQRIKVLNADTQETMMDHALRTISYIADIGNIVVLMARRRMPRSASQDCIETTPGAQEGKKQYKMICHVFESEDAQLIAQSIGQAFSVAYQEFLRANGINPEDLSQKEYSDIINTQEMYNDDLIHFSNSENCKELQLEKHKGEILGVVVVESGWGSILPTVILANMMNGGPAARSGKLSIGDQIMSINGTSLVGLPLATCQGIIKGLKNQTQVKLNIVSCPPVTTVLIKRPDLKYQLGFSVQNGIICSLMRGGIAERGGVRVGHRIIEINGQSVVATAHEKIVQALSNSVGEIHMKTMPAAMFRLLTGQETPLYI

SEQ ID NO: 36 - protein, 737 aa, Homo sapiens
MAHRKLESVGSGMLDHRVRPGPVPHSQEPESEDMELPLEGYVPEGLELAALRPESPAPEEQECHNHSPDGDSSSDYVNNTSEEEDYDEGLPEEEEGITYYIRYCPEDDSYLEGMDCNGEEYLAHSAHPVDTDECQEAVEEWTDSAGPHPHGHEAEGSQDYPDGQLPIPEDEPSVLEAHDQEEDGHYCASKEGYQDYYPEEANGNTGASPYRLRRGDGDLEDQEEDIDQIVAEIKMSLSMTSITSASEASPEHGPEPGPEDSVEACPPIKASCSPSRHEARPKSLNLLPEAKHPGDPQRGFKPKTRTPEERLKWPHEQVCNGLEQPRKQQRSDLNGPVDNNNIPETKKVASFPSFVAVPGPCEPEDLIDGIIFAANYLGSTQLLSERNPSKNIRMMQAQEAVSRVKNSEGDAQTLTEVDLFISTQRIKVLNADTQETMMDHALRTISYIADIGNIVVLMARRRMPRSASQDCIETTPGAQEGKKQYKMICHVFESEDAQLIAQSIGQAFSVAYQEFLRANGINPEDLSQKEYSDIINTQEMYNDDLIHFSNSENCKELQLEKHKGEILGVVVVESGWGSILPTVILANMMNGGPAARSGKLSIGDQIMSINGTSLVGLPLATCQGIIKGLKNQTQVKLNIVSCPPVTTVLIKRPDLKYQLGFSVQNGIICSLMRGGIAERGGVRVGHRIIEINGQSVVATAHEKIVQALSNSVGEIHMKTMPAAMFRLLTGQETPLYI

SEQ ID NO: 37 - protein, 152 aa, Homo sapiens
MPAHSLVMSSPALPAFLLCSTLLVIKMYVVAIITGQVRLRKKAFANPEDALRHGGPQYCRSDPDVERCLRAHRNDMETIYPFLFLGFVYSFLGPNPFVAWMHFLVFLVGRVAHTVAYLGKLRAPIRSVTYTLAQLPCASMALQILWEAARHL

SEQ ID NO: 38 - protein, 1098 aa, Homo sapiens
MGSKERFHWQSHNVKQSGVDDMVLLPQITEDAIAANLRKRFMDDYIFTYIGSVLISVNPFKQMPYFTDREIDLYQGAAQYENPPHIYALTDNMYRNMLIDCENQCVIISGESGAGKTVAAKYIMGYISKVSGGGEKVQHVKDIILQSNPLLEAFGNAKTVRNNNSSRFGKYFEIQFSRGGEPDGGKISNFLLEKSRVVMQNENERNFHIYYQLLEGASQEQRQNLGLMTPDYYYYLNQSDTYQVDGTDDRSDFGETLSAMQVIGIPPSIQQLVLQLVAGILHLGNISFCEDGNYARVESVDLLAFPAYLLGIDSGRLQEKLTSRKMDSRWGGRSESINVTLNVEQAAYTRDALAKGLYARLFDFLVEAINRAMQKPQEEYSIGVLDIYGFEIFQKNGFEQFCINFVNEKLQQIFIELTLKAEQEEYVQEGIRWTPIQYFNNKVVCDLIENKLSPPGIMSVLDDVCATMHATGGGADQTLLQKLQAAVGTHEHFNSWSAGFVIHHYAGKVSYDVSGFCERNRDVLFSDLIELMQTSEQAFLRMLFPEKLDGDKKGRPSTAGSKIKKQANDLVATLMRCTPHYIRCIKPNETKRPRDWEENRVKHQVEYLGLKENIRVRRAGFAYRRQFAKFLQRYAILTPETWPRWRGDERQGVQHLLRAVNMEPDQYQMGSTKVFVKNPESLFLLEEVRERKFDGFARTIQKAWRRHVAVRKYEEMREEASNILLNKKERRRNSINRNFVGDYLGLEERPELRQFLGKRERVDFADSVTKYDRRFKPIKRDLILTPKCVYVIGREKVKKGPEKGQVCEVLKKKVDIQALRGVSLSTRQDDFFILQEDAADSFLESVFKTEFVSLLCKRFEEATRRPLPLTFSDTLQFRVKKEGWGGGGTRSVTFSRGFGDLAVLKVGGRTLTVSVGDGLPKSSKPTRKGMAKGKPRRSSQAPTRAAPAPPRGMDRNGVPPSARGGPLPLEIMSGGGTHRPPRGPPSTSLGASRRPRARPPSEHNTEFLNVPDQGMAGMQRKRSVGQRPVPGVGRPKPQPRTHGPRCRALYQYVGQDVDELSFNVNEVIEILMEDPSGWWKGRLHGQEGLFPGNYVEKI

SEQ ID NO: 39 - protein, 396 aa, Homo sapiens
MWNSSDANFSCYHESVLGYRYVAVSWGVVVAVTGTVGNVLTLLALAIQPKLRTRFNLLIANLTLADLLYCTLLQPFSVDTYLHLHWRTGATFCRVFGLLLFASNSVSILTLCLIALGRYLLIAHPKLFPQVFSAKGIVLALVSTWVVGVASFAPLWPIYILVPVVCTCSFDRIRGRPYTTILMGIYFVLGLSSVGIFYCLIHRQVKRAAQALDQYKLRQASIHSNHVARTDEAMPGRFQELDSRLASGGPSEGISSEPVSAATTQTLEGDSSEVGDQINSKRAKQMAEKSPPEASAKAQPIKGARRAPDSSSEFGKVTRMCFAVFLCFALSYIPFLLLNILDARVQAPRVVHMLAANLTWLNGCINPVLYAAMNRQFRQAYGSILKRGPRSFHRLH

SEQ ID NO: 40 - protein, 227 aa, Homo sapiens
MEKLFNENEGMPSNQGKIDNEEQPPHEGKPEVACILEDKKLENEGNTENTGKRVEEPLKDKEKPESAGKAKGEGKSERKGKSEMQGGSKTEGKPERGGRAEGEGEPDSEREPESEGEPESETRAAGKRPAEDDIPRKAKRKTNKGLAQYLKQYKEAIHDMNFSNEDMIREFDNMARVEDKRRKSKQKLGAFLWMQRNLQDPFYPRGPREFRGGCRAPRRDTEDIPYV

SEQ ID NO: 41 - protein, 540 aa, Homo sapiens
MGPGERAGGGGDAGKGNAAGGGGGGRSATTAGSRAVSALCLLLSVGSAAACLLLGVQAAALQGRVAALEEERELLRRAGPPGALDAWAEPHLERLLREKLDGLAKIRTAREAPSECVCPPGPPGRRGKPGRRGDPGPPGQSGRDGYPGPLGLDGKPGLPGPKGEKGAPGDFGPRGDQGQDGAAGPPGPPGPPGARGPPGDTGKDGPRGAQGPAGPKGEPGQDGEMGPKGPPGPKGEPGVPGKKGDDGTPSQPGPPGPKGEPGSMGPRGENGVDGAPGPKGEPGHRGTDGAAGPRGAPGLKGEQGDTVVIDYDGRILDALKGPPGPQGPPGPPGIPGAKGELGLPGAPGIDGEKGPKGQKGDPGEPGPAGLKGEAGEMGLSGLPGADGLKGEKGESASDSLQESLAQLIVEPGPPGPPGPPGPMGLQGIQGPKGLDGAKGEKGASGERGPSGLPGPVGPPGLIGLPGTKGEKGRPGEPGLDGFPGPRGEKGDRSERGEKGERGVPGRKGVKGQKGEPGPPGLDQPCPVGPDGLPVPGCWHK

SEQ ID NO: 42 - protein, 359 aa, Homo sapiens
MRSIRKRWTICTISLLLIFYKTKEIARTEEHQETQLIGDGELSLSRSLVNSSDKIIRKAGSSIFQHNVEGWKINSSLVLEIRKNILRFLDAERDVSVVKSSFKPGDVIHYVLDRRRTLNISHDLHSLLPEVSPMKNRRFKTCAVVGNSGILLDSECGKEIDSHNFVIRCNLAPVVEFAADVGTKSDFITMNPSVVQRAFGGFRNESDREKFVHRLSMLNDSVLWIPAFMVKGGEKHVEWVNALILKNKLKVRTAYPSLRLIHAVRGYWLTNKVPIKRPSTGLLMYTLATRFCDEIHLYGFWPFPKDLNGKAVKYHYYDDLKYRYFSNASPHRMPLEFKTLNVLHNRGALKLTTGKCVKQ

SEQ ID NO: 43 - protein, 168 aa, Homo sapiens
MRSIRKRWTICTISLLLIFYKTKEIARTEEHQETQLIGDGELSLSRSLVNSSDKIIRKAGSSIFQHNVEGWKINSSLVLEIRKNILRFLDAERDVSVVKSSFKPGDVIHYVLDRRRTLNISHDLHSLLPEVSPMKNRRFKTCAVVGNSGILLDSECGKEIDSHNFVIR

SEQ ID NO: 44 - protein, 467 aa, Homo sapiens [sequence omitted]

SEQ ID NO: 45 - protein, 304 aa, Homo sapiens
MLRGSASSTSMEKAKGKEWTSTEKSREEDQQASNQPNSIALPGTSAKRTKEKMSIKGSKVLCPKKKAEHTDNPRPQKKIPIPPLPSKLPPVNLIHRDILRAWCQQLKLSSKGQKLDAYKRLCAFAYPNQKDFPSTAKEAKIRKSLQKKLKVEKGETSLQSSETHPPEVALPPVGEPPALENSTALLEGVNTVVVTTSAPEALLASWARISARARTPEAVESPQEASGVRWCVVHGKSLPADTDGWVHLQFHAGQAWVPEKQEGRVSALFLLPASNFPPPHLEDNMLCPKCVHRNKVLIKSLQWE

SEQ ID NO: 46 - protein, 205 aa, Homo sapiens
MGTAGAMQLCWVILGFLLFRGHNSQPTMTQTSSSQGGLGGLSLTTEPVSSNPGYIPSSEANRPSHLSSTGTPGAGVPSSGRDGGTSRDTFQTVPPNSTTMSLSMREDATILPSPTSETVLTVAAFGVISFIVILVVVVIILVGVVSLRFKCRKSKESEDPQKPGSSGLSESCSTANGEKDSITLISMKNINMNNGKQSLSAEKVL